(19) United States
(12) Patent Application Publication
(10) Pub. No.: US 2026/0115190 A1
(43) Pub. Date: Apr. 30, 2026

(54) DISPERSIONS OF ETRUMADENANT

(71) Applicant: Arcus Biosciences, Inc., Hayward, CA (US)

(72) Inventors: Balaji Agoram, Burlingame, CA (US); Manshiu Leung, Daly City, CA (US); Isabel Olson, Livermore, CA (US); Yogesh Balasaheb Patil, Danville, CA (US); Andrew M.K. Pennell, San Francisco, CA (US); Leena K. Prasad, Berkeley, CA (US); Lian Zhou, Indianapolis, IN (US); Vahid Zia, Palo Alto, CA (US)

(21) Appl. No.: 19/110,327

(22) PCT Filed: Sep. 13, 2023
(86) PCT No.: PCT/US2023/032644

§ 371 Date: Mar. 10, 2025

Related U.S. Application Data
(60) Provisional application No. 63/375,574, filed on Sep. 14, 2022.
(60) Provisional application No. 63/387,672, filed on Dec. 15, 2022.

(51) Int. Cl. A61K 31/506; A61K 9/16; A61K 9/20; A61K 9/28; A61K 9/48; A61K 31/496

(52) U.S. Cl. CPC A61K 31/506; A61K 9/1635; A61K 9/1652; A61K 9/1694; A61K 9/2009; A61K 9/2013; A61K 9/2018; A61K 9/2027; A61K 9/2054; A61K 9/28; A61K 9/4858; A61K 9/4866; A61K 31/496

(57) ABSTRACT

Solid dispersion comprising 20-40 wt % of etrumadenant and 60-80 wt % of a polymer selected from HPMCAS, PVP-PA, CAP, HPMC E3, HPMCP, PVP and polyvinyl caprolactam-polyvinyl acetate-PEG graft copolymer. A process for preparing said composition by spray drying. Said composition for use in the treatment of cancer.

### CROSS-REFERENCE TO RELATED APPLICATIONS

This application claims benefit to U.S. Provisional Patent Application No. 63/375,574, filed Sep. 14, 2022, and U.S. Provisional Patent Application No. 63/387,672, filed Dec. 15, 2022. The contents of each of the aforementioned applications are incorporated by reference herein in their entireties.

### FIELD

Provided herein are solid and semi-solid dispersions of etrumadenant and compositions thereof.

### BACKGROUND

Adenosine is a purine nucleoside compound comprising a complex of adenine and a ribose sugar molecule (ribofuranose). Adenosine occurs naturally in mammals and plays important roles in several biochemical processes, including energy transfer (as adenosine triphosphate and adenosine monophosphate) and signal transduction (as cyclic adenosine monophosphate). Adenosine also serves in processes associated with vasodilation, including cardiac vasodilation, and acts as a neuromodulator (i.e., it is thought to be involved in promoting sleep). In addition to its involvement in these biochemical processes, adenosine is used as a therapeutic antiarrhythmic agent to treat, for example, supraventricular tachycardia. Tumors evade host responses by inhibiting immune function and promoting tolerance, and adenosine has been shown to play an important role in mediating tumor evasion of the immune system. Adenosine signaling through A_{2A}Rs and A_{2B}Rs, expressed on a variety of immune cell subsets and endothelial cells, has been established as having an important role in protecting tissues during inflammatory responses. As such, under certain conditions adenosine protects tumors from immune destruction (see, e.g., Fishman, P, et al. (2009) Handb Exp Pharmacol 193:399-441).

The adenosine receptors are a class of purinergic G protein-coupled receptors with adenosine as the endogenous ligand. The four types of adenosine receptors in humans are referred to as A₁, A_{2A}, A_{2B}, and A₃. Modulation of A₁ has been proposed for the management and treatment of, for example, neurological disorders, asthma, and heart and renal failure; A_{2A} antagonists have been proposed for the management and treatment of, for example, Parkinson's disease; modulation of A_{2B} has been proposed for the management and treatment of, for example, chronic pulmonary diseases, including asthma; and modulation of A₃ has been proposed for the management and treatment of, for example, asthma and chronic obstructive pulmonary diseases, glaucoma, cancer, and stroke.

Historically, modulators of adenosine receptors have been nonselective. This is acceptable in certain indications, such as where the endogenous agonist adenosine, which acts on all four adenosine receptors in cardiac tissue, is administered parenterally for the treatment of severe tachycardia. However, the use of sub-type selective adenosine receptor agonists and antagonists provides the potential for achieving desired outcomes while minimizing or eliminating adverse effects.

Etrumadenant (also known as AB928), has been reported to be a sub-type selective adenosine receptor antagonist. Etrumadenant is a potent antagonist of A_{2A}R and A_{2B}R with a potency on both receptors of less than 10 nM.

### SUMMARY

Provided herein are solid dispersions comprising about 20% to about 40% (w/w) of etrumadenant and about 60% to about 80% (w/w) of a polymer (w/w) selected from hydroxypropylmethylcellulose acetate succinate (HPMCAS) and copovidone (PVP-VA).

Also provided are dosage forms, granules, compositions, pharmaceutical compositions, and tablets thereof; methods of use thereof; and methods of making solid dispersions.

Other aspects and iterations of the disclosure are provided in more detail below.

### BRIEF DESCRIPTION OF THE DRAWINGS

FIG. 1 shows an x-ray powder diffraction (XRPD) pattern of crystalline etrumadenant.

FIG. 2A shows an illustration of a solvent shift dissolution experiment. API (i.e., starting drug substance) is dissolved in DMSO and then dosed into a solution of FaSSIF (Fasted State Simulated Intestinal Fluid) and polymer. Aliquots of sampler are taken at various time points and analyzed by HPLC.

FIG. 2B is a graph depicting the results of a solvent shift dissolution experiment at 25% drug loading. Etrumadenant solubility (μg/mL) is on the y-axis and time (minutes) is on the x-axis. As summarized in the figure legend, polymers tested included CAP, HPMC E3LV, HPMCAS-H, HPMCAS-L, HPMCAS-M, HPMCP, PVP, PVP-VA, and soluplus. The control sample with no polymer is also graphed.

FIG. 2C is a graph depicting the results of a solvent shift dissolution experiment at 50% drug loading. Etrumadenant solubility (μg/mL) is on the y-axis and time (minutes) is on the x-axis. As summarized in the figure legend, polymers tested included CAP, HPMC E3LV, HPMCAS-H, HPMCAS-L, HPMCAS-M, HPMCP, PVP, PVP-VA, and soluplus. The control sample with no polymer is also graphed.

FIG. 3A and FIG. 3B shows modulated differential scanning calorimetry (MDSC) plots of SDI particles in reversing (FIG. 3A) or non-reversing (FIG. 3B) mode.

FIG. 4 shows XRPD patterns of SDI particles and starting drug substance (i.e., AB928).

FIG. 5 is a graph depicting non-sink dissolution of SDI particles (see figure legend) and starting drug substance (“AB928 API”). The vertical line (denoted as “GASTRIC TRANSFER (0.1N HCl→FaSSIF)” in figure legend) delineates the time of gastric transfer, i.e., the change from 0.1N HCl to FaSSIF.

FIG. 6 shows XRPD patterns of 25:75 AB928:HPMCAS-M SDIs and 40:60 AB928:HPMCAS-M SDIs after 12 weeks on stability at 25° C./60% relative humidity (“RH”) (“25/60”) or 40° cn5% RH (“4075”) in open or closed containers.

FIG. 7 shows XRPD patterns of 25:75 AB928:PVP-VA SDIs and 40:60 AB928:PVP-VA SDIs after 12 weeks on stability at 25° C./60% RH (“25/60”) or 40° C./75% RH (“40/75”) in open or closed containers.

FIG. 8 shows XRPD patterns of 25:75 AB928:HPMCAS-M SDIs, 30:70 AB928:HPMCAS-M SDIs, 35:65 AB928:HPMCAS-M SDIs, and 40:60 AB928:HPMCAS-M SDIs after 6 months at 25° C./60% RH (“25/60”) or 40° cn5% RH (“4015”) on stability in closed containers.

FIG. 9A and FIG. 9B show MDSC plots of 25:75 AB928:HPMCAS-M SDIs, 30:70 AB928:HPMCAS-M SDIs, 35:65 AB928:HPMCAS-M SDIs, and 40:60 AB928:HPMCAS-M SDIs after 6 months on stability at 25° C./60% RH (“25/60”) or 40° C./75% RH (“4015”) in open containers (FIG. 9A) or closed containers (FIG. 9B).

FIG. 10 is a graph depicting non-sink dissolution of 25:75 AB928:HPMCAS-M SDIs, 30:70 AB928:HPMCAS-M SDIs, 35:65 AB928:HPMCAS-M SDIs, and 40:60 AB928:HPMCAS-M SDIs at t=0 d (time of manufacturing). The vertical line (denoted as “GASTRIC TRANSFER (0.1N HCl 4 FaSSIF)” in figure legend) delineates the time of gastric transfer, i.e., the change from 0.1N HCl to FaSSIF.

FIG. 11 is a graph depicting non-sink dissolution of 25:75 AB928:HPMCAS-M SDIs, 30:70 AB928:HPMCAS-M SDIs, 35:65 AB928:HPMCAS-M SDIs, and 40:60 AB928:HPMCAS-M SDIs after 6 months on stability at 25° C./60% RH or 40° C./75% RH in closed containers. The vertical line (denoted as “GASTRIC TRANSFER (0.1N HCl→FaSSIF)” in figure legend) delineates the time of gastric transfer, i.e., the change from 0.1N HCl to FaSSIF.

FIG. 12A shows a graph depicting tabletability, FIG. 12B shows a graph depicting compressibility, FIG. 12C shows a graph depicting compactibility, and FIG. 12D shows a graph depicting disintegration time of 25:75 AB928:HPMCAS-M SDI tablets (“25% HPMCAS-M SDI Tablet”), and 40:60 AB928:HPMCAS-M SDI tablets (“40% HPMCAS-M SDI Tablet”).

FIG. 13A shows a graph depicting tabletability, FIG. 13B shows a graph depicting compressibility, FIG. 13C shows a graph depicting compactibility, and FIG. 13D shows a graph depicting disintegration time of 25:75 AB928:PVP-VA SDI tablets (“25% PVP-VA SDI Tablet”), and 40:60 AB928:PVP-VA SDI tablets (“40% PVP-VA SDI Tablet”).

FIG. 14 is a graph depicting non-sink dissolution of tablets containing 25:75 AB928:HPMCAS-M SDIs or 25:75 AB928:PVP-VA at t=0 or after 3 months (3M) on stability at 25° C./60% RH (“25/60”) or 40° cn5% RH (“40/75”) in closed containers.

FIG. 15 is a graph depicting non-sink dissolution of tablets containing 40:60 AB928:HPMCAS-M SDIs or 40:60 AB928:PVP-VA after 0 or 3 months on stability at 25° C./60% RH (“25/60”) or 40° C./75% RH (“40/75”) in closed containers.

FIG. 16 is a graph depicting non-sink dissolution of tablets containing 25:75 AB928:HPMCAS-M SDIs or 40:60 AB928:HPMCAS-M SDIs after 0 or 12 months on stability at 25° C./60% RH (“25/60”) or 40° cn5% RH (“40/75”) in closed containers.

FIG. 17A is a graph depicting plasma concentration of AB928 (y-axis) over time (x-axis) following oral administration of an AB928 capsule (“capsule control”) or one of three AB928 tablet formulations having HPMCAS SDIs (see figure legend).

FIG. 17B is a graph depicting plasma concentration of AB928 (y-axis) over time (x-axis) following oral administration of an AB928 capsule (“capsule control”) or one of three AB928 tablet formulations having PVP-VA SDIs (see figure legend).

FIG. 18 is a graph depicting plasma etrumadenant box plots, median and Cmax following a single dose of 150 mg of etrumadenant with and without multiple doses of 200 mg itraconazole. In the graph, the upper and lower limits of the box represent the third and first quartiles, respectively, while the midline represents the median. The filled circle represents the mean. The upper and lower whiskers of the boxplot represent the largest and the smallest observed values within 1.5×the interquartile range, respectively. The open circles represent the individual parameter values.

FIG. 19 shows an x-ray powder diffraction (XRPD) pattern of a crystalline form of a phosphate salt of etrumadenant (“Phosphate Salt Form 1”).

FIG. 20 shows an x-ray powder diffraction (XRPD) pattern of a crystalline form of a phosphate salt of etrumadenant (“Phosphate Salt Form II”).

FIG. 21 shows an x-ray powder diffraction (XRPD) pattern of crystalline etrumadenant fumaric acid salt.

### DETAILED DESCRIPTION

The present disclosure provides dispersions comprising etrumadenant, or a pharmaceutically acceptable salt thereof, compositions thereof, and pharmaceutical compositions thereof (such as tablets). It is contemplated that the dispersions, compositions, and pharmaceutical compositions provided herein may exhibit one or more of improved drug solubility, improved bioavailability, and/or elimination of or reduction in food-effect (for example, as compared to crystalline forms of etrumandenant or pharmaceutically acceptable salts thereof). Also provided herein are methods of making the dispersions and methods of use thereof. Dispersions of etrumadenant disclosed herein can be formulated to have a high drug load, for example, up to and including 50% (w/w) of etrumadenant.

### I. Definitions

Unless otherwise defined, all terms of art, notations and other scientific terms or terminology used herein are intended to have the meanings commonly understood by those of skill in the art to which this disclosure pertains. It is understood that aspects of the disclosure described herein include “comprising,” “consisting,” and “consisting essentially of” aspects. The terms “comprising”, “including,” and “having” are intended to be inclusive and mean that there may be additional elements other than the listed elements. The term “consisting essentially of” means that specific further components can be present, namely those not materially affecting the essential characteristics of the compound or composition.

Further, the singular forms “a,” “an,” and “the” include plural references unless the context clearly dictates otherwise. Thus, for example, reference to “a particle” includes reference to one or more particles, and equivalents thereof known to those skilled in the art.

The term “about” is used herein has its original meaning of approximately and is to provide literal support for the exact number that it precedes, as well as a number that is near to or approximately the number that the term precedes. In general, the term “about” refers to the usual error range for the respective value readily known to the skilled person in this technical field. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

Also as used herein, “and/or” refers to and encompasses any and all possible combinations of one or more of the associated listed items, as well as the lack of combinations when interpreted in the alternative (“or”). Similarly, a phrase in the form “AB” or in the form “A and/or B” means (A), (B), or (A and B); a phrase in the form “at least one of A, B, and C” means (A), (B), (C), (A and B), (A and C), (B and C), or (A, B, and C).

The term “% w/w” as used herein refers to the weight of a component based on the total weight of a composition comprising the component. For example, if component A is present in an amount of 50% w/w in a 100 mg composition, component A is present in an amount of 50 mg.

A “Dv50” value refers to the size of 50% of the total volume of material in a sample. A “Dv90” value refers to the size of 90% of the total volume of material in a sample.

The term “amorphous” refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterized by a change of state, typically second order (glass transition).

The term “polymer matrix” as used herein is defined to mean compositions comprising one or more polymers in which the active agent is dispersed or included within the matrix.

The term “pharmaceutically acceptable salt” is meant to include salts of the active compounds which are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, maleic, oxalic, trans-cinnamic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

The term “disintegrant” refers to a substance which, upon addition to a solid preparation, facilitates its break-up or disintegration after administration and permits the release of an active ingredient as efficiently as possible to allow for its rapid dissolution. Non-limiting examples of disintegrants include maize starch, sodium starch glycolate, croscarmellose sodium, crospovidone, microcrystalline cellulose, modified corn starch, sodium carboxymethyl starch, povidone, pregelatinized starch, and alginic acid.

The term “filler” (also known as a diluent) refers to chemical compounds that are used to dilute the compound of interest prior to delivery. Diluents can also serve to stabilize compounds. Non-limiting examples of diluents include starch, saccharides, disaccharides, sucrose, lactose, polysaccharides, cellulose, cellulose ethers, hydroxypropyl cellulose, sugar alcohols, xylitol, sorbitol, maltitol, microcrystalline cellulose, calcium or sodium carbonate, lactose, lactose monohydrate, dicalcium phosphate, cellulose, compressible sugars, dibasic calcium phosphate dehydrate, mannitol, microcrystalline cellulose, and tribasic calcium phosphate.

The term “glidant” as used herein is intended to mean an agent used in tablet and capsule formulations to improve flow-properties during tablet compression and to produce an anti-caking effect. Non-limiting examples of glidants include colloidal silicon dioxide, talc, fumed silica, starch, starch derivatives, and bentonite.

The term “lubricant” refers to an excipient which is added to a powder blend to prevent the compacted powder mass from sticking to the equipment during the tableting or encapsulation process. It aids the ejection of the tablet form the dies, and can improve powder flow. Non-limiting examples of lubricants include magnesium stearate, stearic acid, silica, fats, or talc; and solubilizers such as fatty acids including lauric acid, oleic acid, and C₈/C₁₀ fatty acid.

The term “coating” or “film coating” refers to a thin, uniform, film on the surface of a substrate (e.g. tablet). Film coatings are particularly useful for protecting an active ingredient from photolytic degradation. Non-limiting examples of film coatings include polyvinylalcohol based, hydroxyethylcellulose, hydroxypropylmethylcellulose, sodium carboxymethylcellulose, polyethylene glycol 4000 and cellulose acetate phthalate film coatings.

The term “non-functional coating” refers to a coating that can improve product appearance, handling, and/or stability of the substrate (e.g., tablet), but has no measurable effect on biopharmaceutical properties of the substrate.

The terms “patient” and “subject” are used herein interchangeably to refer to a human or a non-human animal (e.g., a mammal).

The terms “administration,” “administer,” and the like, as they apply to, for example, a subject, cell, tissue, organ, or biological fluid, refer to contact of, for example, a dispersion, composition, or a pharmaceutical composition described herein to the subject, cell, tissue, organ, or biological fluid. In the context of a cell, administration includes contact (e.g., in vitro or ex vivo) of a reagent to the cell, as well as contact of a reagent to a fluid, where the fluid is in contact with the cell.

The terms “treat,” “treating,” treatment,” and the like refer to a course of action that eliminates, reduces, suppresses, mitigates, or ameliorates, or prevents the worsening of, either temporarily or permanently, a disease, disorder, or condition to which the term applies, or at least one of the symptoms associated therewith. Treatment includes, as examples, inhibiting (e.g., arresting the development or further development of the disease, disorder or condition or clinical symptoms association therewith) an active disease, improving the quality of life, and/or prolonging survival of a subject.

The terms “prevent,” “preventing,” “prevention,” “prophylaxis,” and the like refer to a course of action initiated in a manner (e.g., prior to the onset of a disease, disorder, condition, or symptom thereof) so as to prevent, suppress, inhibit or reduce, either temporarily or permanently, a subject's risk of developing a disease, disorder, condition, or the like (as determined by, for example, the absence of clinical symptoms) or delaying the onset thereof, generally in the context of a subject predisposed to having a particular disease, disorder, or condition. In certain instances, the terms also refer to slowing the progression of the disease, disorder, or condition or inhibiting progression thereof to a harmful or otherwise undesired state. Prevention also refers to a course of action initiated in a subject after the subject has been treated for a disease, disorder, condition, or a symptom associated therewith in order to prevent relapse of that disease, disorder, condition, or symptom.

### II. Etrumadenant

Etrumadenant (also known as AB928) is a selective, dual antagonist of the adenosine 2a receptor (A₂ₐR) and the adenosine 2b receptor (A_{2b}R). Its chemical name is 3-[2-amino-6-(1-{[6-(2-hydroxypropan-2-yl)pyridine-2-yl]methyl}-1H-1,2,3-triazol-4-yl)pyrimidin-4-yl]-2-methylbenzonitrile, and its structural formula is shown below:

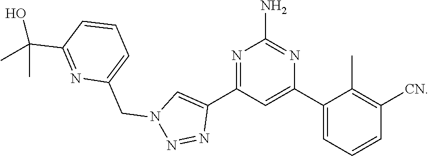 [SMILES: Cc1c(C#N)cccc1-c1cc(-c2cn(Cc3cccc(C(C)(C)O)n3)nn2)nc(N)n1]

Etrumadenant and pharmaceutically acceptable salts thereof can adopt a variety of forms including, but not limited to an amorphous form, a crystalline form, a mixture of crystalline forms, and a mixture of amorphous and crystalline forms. In various embodiments of the present disclosure (e.g., compositions, dispersions, granules, tablets, etc.), etrumadenant or a pharmaceutically acceptable salt thereof may be in an amorphous form, a crystalline form, or any mixture thereof. Crystalline forms of etrumadenant include those described in WO 2020/018680, the disclosure of which is incorporated herein by reference in its entirety. In some embodiments, crystalline etrumadenant may be Form I of WO 2020/018680. In some embodiments, crystalline etrumadenant may be Form II of WO 2020/018680. In some embodiments, crystalline etrumadenant may be Form III of WO 2020/018680.

Methods for making etrumadenant are known in the art. See, for example, WO 2018/136700, WO 2020/018680, and WO 2020/247789, the disclosures of which are incorporated herein by reference in their entirety.

### III. Dispersions of Etrumadenant & Methods of Making Thereof

### Dispersions

Disclosed herein are dispersions of etrumadenant, or a pharmaceutically acceptable salt thereof, in a solid or a semi-solid state at temperatures below 40° C.

A dispersion of etrumadenant, or a pharmaceutically acceptable salt thereof, as used herein, refers to a dispersion (i.e., distribution) of an active agent, such as etrumadenant or a pharmaceutically acceptable salt thereof, in a matrix. As used herein, a “solid dispersion” refers to a dispersion in a solid state at temperatures below 40° C., while a “semi-solid dispersion” refers to a dispersion in a semi-solid state at temperatures below 40° C. In some embodiments, a solid dispersion described herein is in a solid state at 25° C. In some embodiments, a semi-solid dispersion described herein is in a semi-solid state at 25° C.

For the avoidance of doubt, a semi-solid state is a state that lies between a solid and a liquid. While similar to solids in some respects, such as having the ability to support their own weight and hold their shapes, a semi-solid also shares some properties of liquids, such as conforming in shape to something applying pressure to it and the ability to flow under pressure. Dispersions of the present disclosure can also be characterized, for example, by MDSC, XRPD, scanning electron microscopy (SEM), non-sink dissolution, Karl-Fischer titration, assay, impurity, and non-sink dissolution.

The matrix of the dispersion comprises at least one hydrophilic polymer (e.g., 1, 2, 3, 4, or more polymers; or 1 to 4 polymers; or 1 to 3 polymers). In some embodiments, the matrix comprises or consists essentially of one polymer. In some embodiments, the matrix comprises or consists essentially of a mixture of polymers. The polymer may be synthetic or natural. Non-limiting examples of suitable polymers may include polyethylene glycol polymers (e.g., PEG 400, PEG 1500, PEG 4000, etc.), cellulose-based polymers (e.g., HPMC, HPMCAS, HPMCP, CAP, etc.) and vinyl-pyrrolidone-based polymers (e.g., povidone (“PVP”), copovidone (“PVP-VA”), Soluplus®, etc.).

The matrix may further comprise one or more additional component, including but not limited to one or more antioxidant, one or more surfactant, one or more solubilizer, or any combination thereof. Non-limiting examples of suitable antioxidants include ascorbic acid, BHT, tocofersolan, and Vitamin E. Non-limiting examples of suitable surfactants include inulin, inutec, poloxamer, tocofersolan, Compritol® surfactants (e.g., Compritol® 888 ATO, etc.), Gelucire® surfactants (e.g., Gelucire® 44/14 etc.), and Kolliphor® surfactants (e.g., Kolliphor® HS 15, Kolliphor® RH40, etc.), polysorbates (e.g., polysorbate 20, polysorbate 80, etc.), egg lecithin, and soy lecithin. Solvent(s) used in the preparation of the dispersion and/or water may also be present in the dispersion, though typically these are present in minor amounts (e.g., preferably less than 5%, less than 4%, less than 3%, or less than 2% by weight).

Accordingly, a dispersion of the present disclosure comprises etrumadenant or a pharmaceutically acceptable salt thereof and a polymer. Further, in some embodiments, a dispersion of the present disclosure may consist essentially of (i) etrumadenant or a pharmaceutically acceptable salt thereof and (ii) a polymer, a mixture of polymers, or a mixture of polymer(s) and surfactant(s). For the avoidance of doubt, a dispersion consisting essentially of (A) and (B) allows for the presence of materials not affecting the essential characteristics of the dispersion, such as, minor impurities, related substances of A and/or B, residual solvent, and water.

In some embodiments, the present disclosure provides a solid dispersion comprising etrumadenant or a pharmaceutically acceptable salt thereof and a polymer. In some embodiments, the solid dispersion comprises (i) etrumadenant or a pharmaceutically acceptable salt thereof and (ii) a polymer, a mixture of polymers, or a mixture of polymer(s) and surfactant(s). In some embodiments, the solid dispersion consists essentially of (i) etrumadenant or a pharmaceutically acceptable salt thereof and (ii) a polymer, a mixture of polymers, or a mixture of polymer(s) and surfactant(s). Non-limiting examples of suitable polymers include cellulose-based polymers (e.g., HPMC, HPMCAS, HPMCP, CAP, etc.) and vinyl-pyrrolidone-based polymers (e.g., povidone, copovidone, soluplus, etc.). In some embodiments, a solid dispersion of the present disclosure comprises or consists essentially of etrumadenant or a pharmaceutically acceptable salt thereof and a polymer selected from HPMCAS and copovidone.

In another aspect, the present disclosure provides a semi-solid dispersion comprising etrumadenant or a pharmaceutically acceptable salt thereof and a polymer. In some embodiments, the semi-solid dispersion comprises (i) etrumadenant or a pharmaceutically acceptable salt thereof and (ii) a polymer, a mixture of polymers, or a mixture of polymer(s) and surfactant(s). In some embodiments, the semi-solid dispersion consists essentially of (i) etrumadenant or a pharmaceutically acceptable salt thereof and (ii) a polymer, a mixture of polymers, or a mixture of polymer(s) and surfactant(s). Non-limiting examples of suitable polymers include cellulose-based polymers (e.g., HPMC, HPMCAS, HPMCP, CAP, etc.) and vinyl-pyrrolidone-based polymers (e.g., povidone, copovidone, soluplus, etc.). In a specific example, a solid dispersion of the present disclosure comprises or consists essentially of etrumadenant or a pharmaceutically acceptable salt thereof and one or more polyethylene glycol polymer.

Provided herein are solid dispersions comprising about 25% to about 50% (w/w) of etrumadenant and about 50% to about 75% (w/w) of a polymer. In some embodiments, the polymer is selected from hydroxypropylmethylcellulose acetate succinate (HPMCAS), copovidone, cellulose acetate phthalate (CAP), hydroxypropyl methylcellulose E3 (HPMC E3), hydroxypropyl methylcellulose phthalate (HPMCP), polyvinylpyrrolidone (PVP), and polyvinyl caprolactam-polyvinyl acetate-polyethylene glycol graft copolymer (Soluplus®).

Provided herein are solid dispersions comprising about 20% to about 40% (w/w) of etrumadenant and about 60% to about 80% (w/w) of a polymer selected from hydroxypropylmethylcellulose acetate succinate (HPMCAS), copovidone (PVP-VA), cellulose acetate phthalate (CAP), hydroxypropyl methylcellulose E3 (HPMC E3), hydroxypropyl methylcellulose phthalate (HPMCP), polyvinylpyrrolidone (PVP), and polyvinyl caprolactam-polyvinyl acetate-polyethylene glycol graft copolymer (Soluplus®).

Provided herein are solid dispersions comprising about 25% to about 50% (w/w) of etrumadenant and about 50% to about 75% (w/w) of a polymer (w/w) selected from hydroxypropylmethylcellulose acetate succinate (HPMCAS) and copovidone.

Provided herein are solid dispersions comprising about 25% to about 40% (w/w) of etrumadenant and about 60% to about 75% (w/w) of a polymer (w/w) selected from hydroxypropylmethylcellulose acetate succinate (HPMCAS) and copovidone.

Provided herein are solid dispersions comprising about 20% to about 40% (w/w) of etrumadenant and about 60% to about 80% (w/w) of a polymer (w/w) selected from hydroxypropylmethylcellulose acetate succinate (HPMCAS) and copovidone.

Provided herein are solid dispersions comprising about 35% to about 50% (w/w) of etrumadenant, or a pharmaceutically acceptable salt thereof, and about 65% to about 50% (w/w) of a polymer (w/w) selected from hydroxypropylmethylcellulose acetate succinate (HPMCAS) and copovidone.

In some embodiments, such solid dispersions comprise about 25% to about 40% (w/w) of etrumadenant.

In some embodiments, the solid dispersion comprises about 25% to about 35% (w/w) of etrumadenant; or about 25% to about 30% (w/w) of etrumadenant. In some embodiments, the solid dispersions comprise about 25% to about 35% (w/w) of etrumadenant. In some embodiments, the solid dispersions comprise about 25% to about 30% (w/w) of etrumadenant.

In some embodiments, the solid dispersion comprises about 22.5% to about 27.5% (w/w) of etrumadenant; or about 27% to about 33% (w/w) of etrumadenant; or about 31.5% to about 38.5% (w/w) of etrumadenant; or about 36% to about 44% (w/w) of etrumadenant. In some embodiments, the solid dispersion comprises about 22.5% to about 27.5% (w/w) of etrumadenant; or about 27% to about 33% (w/w) of etrumadenant; or about 31.5% to about 38.5% (w/w) of etrumadenant.

In some embodiments, the solid dispersions comprise about 22.5% to about 27.5% (w/w) of etrumadenant. In some embodiments, the solid dispersions comprise about 27% to about 33% (w/w) of etrumadenant. In some embodiments, the solid dispersions comprise about 31.5% to about 38.5% (w/w) of etrumadenant. In some embodiments, the solid dispersions comprise about 36% to about 44% (w/w) of etrumadenant.

In some embodiments, the solid dispersions comprise about 20% (w/w) of etrumadenant. In some embodiments, the solid dispersions comprise about 22.5% (w/w) of etrumadenant. In some embodiments, the solid dispersions comprise about 25% (w/w) of etrumadenant. In some embodiments, the solid dispersions comprise about 27% (w/w) of etrumadenant. In some embodiments, the solid dispersions comprise about 27.5% (w/w) of etrumadenant. In some embodiments, the solid dispersions comprise about 30% (w/w) of etrumadenant. In some embodiments, the solid dispersions comprise about 31.5% (w/w) of etrumadenant. In some embodiments, the solid dispersions comprise about 33% (w/w) of etrumadenant. In some embodiments, the solid dispersions comprise about 32.5% (w/w) of etrumadenant. In some embodiments, the solid dispersions comprise about 35% (w/w) of etrumadenant. In some embodiments, the solid dispersions comprise about 36% (w/w) of etrumadenant. In some embodiments, the solid dispersions comprise about 37.5% (w/w) of etrumadenant. In some embodiments, the solid dispersions comprise about 38.5% (w/w) of etrumadenant. In some embodiments, the solid dispersions comprise about 40% (w/w) of etrumadenant. In some embodiments, the solid dispersions comprise about 42.5% (w/w) of etrumadenant. In some embodiments, the solid dispersions comprise about 44% (w/w) of etrumadenant. In some embodiments, the solid dispersions comprise about 45% (w/w) of etrumadenant.

In some embodiments, the solid dispersion comprises about 25% to about 35% (w/w) of etrumadenant and about 65% to about 75% of the polymer; or about 25% to about 30% (w/w) of etrumadenant and about 70% to about 75% (w/w) of the polymer. In some embodiments, the solid dispersion comprises about 25% to about 35% (w/w) of etrumadenant and about 65% to about 75% of the polymer. In some embodiments, the solid dispersion comprises about 25% to about 30% (w/w) of etrumadenant and about 70% to about 75% (w/w) of the polymer.

In some embodiments, the solid dispersion comprises about 25% of etrumadenant and about 75% (w/w) of the polymer. In some embodiments, the solid dispersion comprises about 30% of etrumadenant and about 70% (w/w) of the polymer. In some embodiments, the solid dispersion comprises about 35% of etrumadenant and about 65% (w/w) of the polymer. In some embodiments, the solid dispersion comprises about 40% of etrumadenant and about 60% (w/w) of the polymer.

In some embodiments, the solid dispersion is characterized by a single glass transition temperature (T_{g}). In some embodiments, the solid dispersion is characterized by glass transition temperature (T_{g}) from about 75° C. to about 100° C.; and/or a degree of crystallinity that is no more than 5% or no more than 1%.

In some embodiments, the solid dispersion is characterized by glass transition temperature (T_{g}) from about 75° C. to about 100° C.; or a degree of crystallinity that is no more than 5%. In some embodiments, the solid dispersion is characterized by glass transition temperature (T_{g}) from about 75° C. to about 100° C.; or a degree of crystallinity that is no more than 3%. In some embodiments, the solid dispersion is characterized by glass transition temperature (T_{g}) from about 75° C. to about 100° C.; or a degree of crystallinity that is no more than 1%.

In some embodiments, the solid dispersion is characterized by glass transition temperature (T_{g}) from about 50° C. to about 150° C. In some embodiments, the solid dispersion is characterized by glass transition temperature (T_{g}) from about 60° C. to about 125° C. In some embodiments, the solid dispersion is characterized by glass transition temperature (T_{g}) from about 75° C. to about 125° C. In some embodiments, the solid dispersion is characterized by glass transition temperature (T_{g}) from about 65° C. to about 115° C. In some embodiments, the solid dispersion is characterized by glass transition temperature (T_{g}) from about 70° C. to about 100° C. In some embodiments, the solid dispersion is characterized by glass transition temperature (T_{g}) from about 75° C. to about 90° C. In some embodiments, the solid dispersion is characterized by glass transition temperature (T_{g}) from about 75° C. to about 85° C. In some embodiments, the solid dispersion is characterized by glass transition temperature (T_{g}) from about 72° C. to about 83° C. T_{g} may be determined by modulated differential scanning calorimetry (MDSC).

In some embodiments, the solid dispersion is characterized by a degree of crystallinity that is no more than 5%, no more than 4%, no more than 3%, no more than 2%, or no more than 1%. In some embodiments, the solid dispersion is characterized by a degree of crystallinity that is no more than 5%. In some embodiments, the solid dispersion is characterized by a degree of crystallinity that is no more than 3%. In some embodiments, the solid dispersion is characterized by a degree of crystallinity that is no more than 1%. In some embodiments, the degree of crystallinity refers to amount of crystalline etrumadenant to total SDI.

Degree of crystallinity may be determined, for example, by x-ray powder diffraction (XRPD). In some embodiments, the solid dispersion is characterized by a degree of crystallinity that is below a limit of quantitation (LOQ) by XRPD. In some embodiments, the solid dispersion is characterized by a degree of crystallinity that is below a limit of detection (LOD) by XRPD. In some embodiments, wherein the degree of crystallinity is determined by XRPD, the degree of crystallinity refers to amount of crystalline etrumadenant to total API.

In some embodiments, the solid dispersion is characterized by a single melting temperature (Tₘ); and/or a diffraction pattern by x-ray powder diffraction (XRPD) that is absent of discrete peaks. In some embodiments, the solid dispersion is characterized by a single melting temperature (Tₘ); or a diffraction pattern by x-ray powder diffraction (XRPD) that is absent of discrete peaks.

In some embodiments, the polymer is HPMCAS. In some embodiments, the polymer is copovidone.

In some embodiments, the solid dispersion comprises about 25% to about 35% (w/w) of etrumadenant and about 65% to about 75% of copovidone; or about 25% to about 30% (w/w) of etrumadenant and about 70% to about 75% (w/w) of copovidone. In some embodiments, the solid dispersion comprises about 25% to about 35% (w/w) of etrumadenant and about 65% to about 75% of copovidone. In some embodiments, the solid dispersion comprises about 25% to about 30% (w/w) of etrumadenant and about 70% to about 75% (w/w) of copovidone.

Some embodiments provide for a solid dispersion comprising about 25% to about 40% (w/w) of etrumadenant and about 60% to about 75% (w/w) of hydroxypropylmethylcellulose acetate succinate (HPMCAS).

Some embodiments provide for a solid dispersion comprising about 25% to about 35% (w/w) of etrumadenant and about 65% to about 75% (w/w) of hydroxypropylmethylcellulose acetate succinate (HPMCAS).

Some embodiments provide for a solid dispersion comprising about 25% to about 40% (w/w) of etrumadenant and about 60% to about 75% (w/w) of hydroxypropylmethylcellulose acetate succinate (HPMCAS),
wherein the solid dispersion is characterized by a single glass transition temperature (T_{g}) from about 75° C. to about 85° C.; or a degree of crystallinity that is no more than 5% or no more than 1%.

Some embodiments provide for a solid dispersion comprising about 25% to about 40% (w/w) of etrumadenant and about 60% to about 75% (w/w) of hydroxypropylmethylcellulose acetate succinate (HPMCAS),
wherein the solid dispersion is characterized by a single glass transition temperature (Tr) from about 75° C. to about 85° C.; or a degree of crystallinity that is no more than 5%.

Some embodiments provide for a solid dispersion comprising about 25% to about 40% (w/w) of etrumadenant and about 60% to about 75% (w/w) of hydroxypropylmethylcellulose acetate succinate (HPMCAS),
wherein the solid dispersion is characterized by a single glass transition temperature (T_{g}) from about 75° C. to about 85° C.; or a degree of crystallinity that is no more than 3%.

Some embodiments provide for a solid dispersion comprising about 25% to about 40% (w/w) of etrumadenant and about 60% to about 75% (w/w) of hydroxypropylmethylcellulose acetate succinate (HPMCAS),
wherein the solid dispersion is characterized by a single glass transition temperature (T_{g}) from about 75° C. to about 85° C.; or a degree of crystallinity that is no more than 1%.

Some embodiments provide for a solid dispersion comprising about 25% to about 35% (w/w) of etrumadenant and about 65% to about 75% (w/w) of hydroxypropylmethylcellulose acetate succinate (HPMCAS),
wherein the solid dispersion is characterized by a single glass transition temperature (T_{g}) from about 75° C. to about 85° C.; or a degree of crystallinity that is no more than 5% or no more than 1%.

Some embodiments provide for a solid dispersion comprising about 25% to about 35% (w/w) of etrumadenant and about 65% to about 75% (w/w) of hydroxypropylmethylcellulose acetate succinate (HPMCAS),
wherein the solid dispersion is characterized by a single glass transition temperature (T_{g}) from about 75° C. to about 85° C.; or a degree of crystallinity that is no more than 5%.

Some embodiments provide for a solid dispersion comprising about 25% to about 35% (w/w) of etrumadenant and about 65% to about 75% (w/w) of hydroxypropylmethylcellulose acetate succinate (HPMCAS),
wherein the solid dispersion is characterized by a single glass transition temperature (T_{g}) from about 75° C. to about 85° C.; or a degree of crystallinity that is no more than 3%.

Some embodiments provide for a solid dispersion comprising about 25% to about 35% (w/w) of etrumadenant and about 65% to about 75% (w/w) of hydroxypropylmethylcellulose acetate succinate (HPMCAS),
wherein the solid dispersion is characterized by a single glass transition temperature (T_{g}) from about 75° C. to about 85° C.; or a degree of crystallinity that is no more than 1%.

In some embodiments, the solid dispersion comprises about 25% to about 35% (w/w) of etrumadenant and about 65% to about 75% of HPMCAS; or about 25% to about 30% (w/w) of etrumadenant and about 70% to about 75% (w/w) of HPMCAS. In some embodiments, the solid dispersion comprises about 25% to about 35% (w/w) of etrumadenant and about 65% to about 75% of HPMCAS. In some embodiments, the solid dispersion comprises about 25% to about 30% (w/w) of etrumadenant and about 70% to about 75% (w/w) of HPMCAS.

In some embodiments, the solid dispersion comprises about 22.5% to about 27.5% (w/w) of etrumadenant and about 65% to about 75% of HPMCAS. In some embodiments, the solid dispersion comprises about 27% to about 33% (w/w) of etrumadenant and about 65% to about 75% (w/w) of HPMCAS. In some embodiments, the solid dispersion comprises about 31.5% to about 38.5% (w/w) of etrumadenant and about 65% to about 75% (w/w) of HPMCAS. In some embodiments, the solid dispersion comprises about 36% to about 44% (w/w) of etrumadenant and about 65% to about 75% (w/w) of HPMCAS.

In some embodiments, the solid dispersion comprises about 22.5% to about 27.5% (w/w) of etrumadenant and about 70% to about 75% (w/w) of HPMCAS. In some embodiments, the solid dispersion comprises about 27% to about 33% (w/w) of etrumadenant and about 70% to about 75% (w/w) of HPMCAS. In some embodiments, the solid dispersion comprises about 31.5% to about 38.5% (w/w) of etrumadenant and about 70% to about 75% (w/w) of HPMCAS. In some embodiments, the solid dispersion comprises about 36% to about 44% (w/w) of etrumadenant and about 70% to about 75% (w/w) of HPMCAS.

In some embodiments, the solid dispersion comprises about 25% (w/w) of etrumadenant and about 75% (w/w) of HPMCAS. In some embodiments, the solid dispersion comprises about 30% (w/w) of etrumadenant and about 70% (w/w) of HPMCAS. In some embodiments, the solid dispersion comprises about 35% (w/w) of etrumadenant and about 65% (w/w) of HPMCAS. In some embodiments, the solid dispersion comprises about 40% (w/w) of etrumadenant and about 60% (w/w) of HPMCAS.

In some embodiments, HPMCAS may be any commercially available grade of HPMCAS. In some embodiments, the HPMCAS comprises an acetyl content of about 5% to about 14%, a succinyl content of about 4% to about 18%, a methoxyl content of about 20% to about 26%, and a hydroxypropoxy content of about 5% to about 10%. In some embodiments, the HPMCAS comprises an acetyl content of about 7% to about 11%, a succinyl content of about 10% to about 14%, a methoxyl content of about 21% to about 25%, and a hydroxypropoxy content of about 5% to about 9%.

In some embodiments, the solid dispersion is characterized by a single Ts. In some embodiments, a solid dispersion comprising etrumadenant and HPMCAS is characterized by a single glass transition temperature (T_{g}) from about 75° C. to about 85° C.; and/or a degree of crystallinity that is no more than 5% or no more than 1%. In some embodiments, a solid dispersion comprising etrumadenant and HPMCAS is characterized by a single glass transition temperature (T_{g}) from about 75° C. to about 85° C.; or a degree of crystallinity that is no more than 5%. In some embodiments, a solid dispersion comprising etrumadenant and HPMCAS is characterized by a single glass transition temperature (T_{g}) from about 75° C. to about 85° C.; or a degree of crystallinity that is no more than 3%. In some embodiments, a solid dispersion comprising etrumadenant and HPMCAS is characterized by a single glass transition temperature (T_{g}) from about 75° C. to about 85° C.; or a degree of crystallinity that is no more than 1%. In some embodiments, the solid dispersion is characterized by no other melt or crystallization events; and/or a diffraction pattern by XRPD that is absent of discrete peaks. In some embodiments, the solid dispersion is characterized by no other melt or crystallization events; or a diffraction pattern by XRPD that is absent of discrete peaks.

In some embodiments, a solid dispersion as described herein has at least a 3-fold increase in AUC₃₅₋₂₁₀ FaSSIF (min*μgA/mL) compared to crystalline etrumadenant by non-sink dissolution testing. Non-sink dissolution testing may be carried out as described herein. In some embodiments, a solid dispersion as described herein has about a 4-fold, 5-fold, 6-fold, or 7-fold increase in AUC₃₅₋₂₁₀ FaSSIF (min*μgA/mL) compared to crystalline etrumadenant by non-sink dissolution testing.

In some embodiments, a solid dispersion as described herein has a total impurity amount of less than 2% (% area), measured by HPLC. In some embodiments, a solid dispersion as described herein has a total impurity amount of less than 1% (% area), measured by HPLC. Analysis of impurity by HPLC may be carried out as described herein. In some embodiments, the solid dispersion or tablet has a total impurity amount of about 1.9%, about 1.8%, about 1.7%, about 1.6%, about 1.5%, about 1.4%, about 1.3%, about 1.2%, about 1.1%, about 1.0%, about 0.9%, about 0.8%, about 0.7%, about 0.6%, about 0.5%, about 0.4%, about 0.3%, about 0.2%, about 0.1% (% area), measured by HPLC. In some embodiments, the tablet has a total impurity amount of less than 1% (% area), measured by HPLC. In some embodiments, the solid dispersion or tablet has a total impurity amount of less than 0.5% (% area), measured by HPLC.

Some embodiments provide for solid dispersions that are physically stable, chemically stable, and/or have stable in vitro dissolution properties upon storage at 25° C. and 60% relative humidity (long term stability testing) or 40° C. and 75% relative humidity (accelerated stability testing) in a closed or open packaging for 3 months or more (e.g., 3, 6, 9, 12, 15, 18, 21, 24, 27, 30, 33, 36 months). Stability may be evaluated by a variety of analytical methods including XRPD, MDSC, assay of etrumadenant and impurities by HPLC, and non-sink dissolution testing.

In some embodiments, upon storage at 25° C. and 60% relative humidity and/or at 40° C. and 75% relative humidity, in a closed or open packaging for 3 months or more, the solid dispersion is characterized by (i) a single glass transition temperature (T_{g}) from about 75° C. to about 85° C. or another T_{g} as described herein, measured by MDSC, (ii) no other melt or crystallization events measured by MDSC. (iii) a diffraction pattern by XRPD that is absent of discrete peaks, (iv) a degree of crystallinity no more than 5%, or no more than 1%, or below a LOQ or LOD by XRPD, or (v) any combination of (i) to (iv).

In some embodiments, upon storage at 25° C. and 60% relative humidity and/or at 40° C. and 75% relative humidity, in a closed or open packaging for 3 months or more, the amount of etrumadenant measured by non-sink dissolution testing at 210 minutes (C₂₁₀; μgA/mL) differs by no more than about 25% of the value measured at 0 months. In some embodiments, upon storage at 25° C. and 60% relative humidity and/or at 40° C. and 75% relative humidity, in a closed or open packaging for 3 months or more, the amount of etrumadenant measured by non-sink dissolution testing at 210 minutes (C₂₁₀; μgA/mL) differs by no more than about 15% of the value measured at 0 months. In some embodiments, upon storage at 25° C. and 60% relative humidity and/or at 40° C. and 75% relative humidity, in a closed or open packaging for 3 months or more, the amount of etrumadenant measured by non-sink dissolution testing at 210 minutes (C₂₁₀; μgA/mL) differs by no more than about 12% of the value measured at 0 months. In some embodiments, upon storage at 25° C. and 60% relative humidity and/or at 40° C. and 75% relative humidity, in a closed or open packaging for 3 months or more, the amount of etrumadenant measured by non-sink dissolution testing at 210 minutes (C₂₁₀; μgA/mL) differs by no more than 10% of the value measured at 0 months. In some embodiments, upon storage at 25° C. and 60% relative humidity and/or at 40° C. and 75% relative humidity, in a closed or open packaging for 3 months or more, the amount of etrumadenant measured by non-sink dissolution testing at 210 minutes (C₂₁₀; gA/mL) differs by no more than 5% of the value measured at 0 months.

In some embodiments, upon storage at 25° C. and 60% relative humidity and/or at 40° C. and 75% relative humidity, in a closed or open packaging for 3 months or more, the amount of etrumadenant in FaSSIF as measured by AUC_{35-210 FassIF} (min*μgA/mL) differs by no more than about 25% of the value measured at 0 months. In some embodiments, upon storage at 25° C. and 60% relative humidity and/or at 40° C. and 75% relative humidity, in a closed or open packaging for 3 months or more, the amount of etrumadenant in FaSSIF as measured by AUC_{35-210 FaSSIF} (min*μgA/mL) differs by no more than about 15% of the value measured at 0 months. In some embodiments, upon storage at 25° C. and 60% relative humidity and/or at 40° C. and 75% relative humidity, in a closed or open packaging for 3 months or more, the amount of etrumadenant in FaSSIF as measured by AUC_{35-210 FaSSIF} (min*μgA/mL) differs by no more than about 10% of the value measured at 0 months. In some embodiments, upon storage at 25° C. and 60% relative humidity and/or at 40° C. and 75% relative humidity, in a closed or open packaging for 3 months or more, the amount of etrumadenant in FaSSIF as measured by AUC_{35-210 FaSSIF} (min*μgA/mL) differs by no more than about 5% of the value measured at 0 months.

In some embodiments, upon storage of the solid dispersion at 25° C. and 60% relative humidity in a closed packaging for 6 months, 12 months, 18 months, 24 months, or 36 months:
(i) the amount of etrumadenant in the solid dispersion is about 90% to about 110% of the value measured at 0 months;
(ii) the solid dispersion has a total impurity amount of less than or equal to 2%, or less than or equal to 1% (% area), measured by high-performance liquid chromatography (HPLC);
(iii) the solid dispersion is characterized by a single T_{g}, and optionally a single melting temperature (Tₘ), by modulated differential scanning calorimetry (MDSC);
(iv) the solid dispersion is characterized by a diffraction pattern by XRPD that is absent of discrete peaks;
(v) the amount of etrumadenant measured at 210 minutes by non-sink dissolution testing (C₂₁₀ (μgA/mL) or AUC_{35-210 FaSSIF} (min*μgA/mL)) differs by no more than 15% of the value measured at 0 months; or
(vi) any combination of (i) to (v).

In some embodiments upon storage of the solid dispersion at 25° C. and 60% relative humidity in a closed packaging for 6 months, 12 months, 18 months, 24 months, or 36 months:
(i) the amount of etrumadenant in the solid dispersion is about 90% to about 110% of the value measured at 0 months;
(ii) the solid dispersion has a total impurity amount of less than or equal to 2%, or less than or equal to 1% (% area), measured by high-performance liquid chromatography (HPLC);
(iii) the solid dispersion is characterized by a single T_{g}, and optionally a single melting temperature (Tₘ), by modulated differential scanning calorimetry (MDSC);
(iv) the solid dispersion is characterized by a diffraction pattern by XRPD that is absent of discrete peaks; or
(v) any combination of (i) to (iv).

In some embodiments, provided herein is a sample comprising one or more dispersions as described herein. In some embodiments, provided herein is a sample comprising one or more solid dispersions as described herein. Such samples may exhibit one or more characteristics such as those described herein for a solid dispersion/dispersions described herein.

Some embodiments provide for particles, wherein such particles comprise solid dispersion(s) as described herein. Such particles may exhibit one or more characteristics such as those described herein for a solid dispersion/dispersions described herein.

Solid dispersions described herein may be made according to methods described herein. In some embodiments, solid dispersions described herein are formed by spray-drying.

Some embodiments provide for spray dried particles, wherein such particles are formed by spray drying and comprise solid dispersion(s) as described herein. Such spray dried particles may exhibit one or more characteristics such as those described herein for a solid dispersion/dispersions described herein.

In some embodiments, spray-dried particles comprise a solid dispersion as described herein, wherein the spray-dried particles are characterized by a Dv90 of less than 150 μm or a Dv50 of less than 50 μm. In some embodiments, the spray-dried particles are characterized by a Dv90 of less than 150 μm. In some embodiments, the spray-dried particles are characterized by a Dv50 of less than 50 μm. In some embodiments, the spray-dried particles are characterized by a Dv90 of about 80 μm to about 130 μm; and/or a Dv50 of about 25 μm to about 40 μm. In some embodiments, the spray-dried particles are characterized by a Dv90 of about 80 μm to about 130 μm. In some embodiments, the spray-dried particles are characterized by a Dv50 of about 25 μm to about 40 μm.

In some embodiments, provided herein is a sample comprising one or more particles as described herein. Such samples may exhibit one or more characteristics such as those described herein for a solid dispersion/dispersions as described herein and/or particles as described herein.

### Methods of Making Dispersions

Provided herein are methods of making dispersions as described herein.

In some embodiments, dispersions as described herein may be made according to methods known in the art. Non-limiting examples known in the art for preparing solid dispersions include, but are not limited to spray-drying, melt-extrusion, lyophilization, and solution-evaporation. In some embodiments, provided herein is a process for preparing a solid dispersion comprising etrumadenant, the process comprising: mixing etrumadenant, a polymer as described herein, and a solvent to produce a spray solution; and spray drying the spray solution to produce the solid dispersion. The term, “spray solution,” as used herein, may also be referred to as “solution” or “feed solution.”

Some embodiments provide for a process for preparing a solid dispersion comprising etrumadenant, the process comprising:
mixing etrumadenant, a polymer selected from HPMCAS and copovidone, and a solvent to produce a spray solution; and
spray drying the spray solution to produce the solid dispersion.

In some embodiments, the solid dispersion undergoes further drying to produce a dried powder with a water content of less than about 1% (w/w).

Some embodiments provide for a process for preparing spray dried particles comprising a solid dispersion as described herein, the process comprising:
mixing etrumadenant, a polymer selected from HPMCAS and copovidone, and a solvent to produce a spray solution; and
spray drying the spray solution to produce the spray dried particles.

In some embodiments, the weight ratio of etrumadenant to polymer is 25:75 to 40:60.

Some embodiments provide for a process for preparing spray dried particles comprising a solid dispersion as described herein, the process comprising:
mixing etrumadenant, HPMCAS, and a solvent to produce a spray solution; and
spray drying the spray solution to produce the spray dried particles.

In some embodiments, the weight ratio of etrumadenant to HPMCAS is 25:75 to 40:60.

In some embodiments, the spray solution is prepared at 8% to 13% solids loading.

In some embodiments, the spray dried particles undergo further drying to produce a dried powder with a water content of less than about 1% (w/w).

In some embodiments, the spray drying step comprises atomizing the spray solution into a drying chamber that has an outlet temperature of about 38° C. to about 46° C., a gas to liquid ratio of about 0.5 to about 0.7, and a relative saturation (total) of about 15% to about 23%.

### IV. Compositions and Pharmaceutical Compositions

Provided herein are compositions comprising a semi-solid or solid dispersion as described herein, particles as described herein, spray dried particles as described herein, or samples thereof.

The dispersions of etrumadenant described herein may be in the form of compositions suitable for administration to a subject.

Some embodiments provide for a pharmaceutical composition comprising a dispersion of etrumadenant described herein and one or more pharmaceutically acceptable or physiologically acceptable diluents, carriers or excipients.

In some embodiments, etrumadenant, or a pharmaceutically acceptable salt thereof, is present in a therapeutically acceptable amount. The pharmaceutical compositions may be used in the methods of the present disclosure; thus, for example, the pharmaceutical compositions can be administered to a subject in order to practice the therapeutic and prophylactic methods and uses described herein.

The pharmaceutical compositions of the present disclosure can be formulated to be compatible with the intended method or route of administration; exemplary routes of administration are set forth herein. Furthermore, the pharmaceutical compositions may be used in combination with other therapeutically active agents or compounds as described herein in order to treat or prevent the diseases, disorders, and conditions as contemplated by the present disclosure.

The pharmaceutical compositions comprising a dispersion described herein may be in a form suitable for oral use, for example, as tablets, capsules, troches, lozenges, aqueous or oily suspensions, dispersible powders or granules, emulsions, hard or soft capsules, or syrups, solutions, microbeads or elixirs. Pharmaceutical compositions intended for oral use may be prepared according to any method known to the art for the manufacture of pharmaceutical compositions, and such compositions may contain one or more agents such as, for example, sweetening agents, flavoring agents, coloring agents, and preserving agents in order to provide pharmaceutically elegant and palatable preparations. Tablets, capsules, and the like contain the active ingredient in admixture with non-toxic pharmaceutically acceptable excipients which are suitable for the manufacture of tablets. These excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

The tablets, capsules, and the like suitable for oral administration may be uncoated or coated by known techniques to delay disintegration and absorption in the gastrointestinal tract and thereby provide a sustained action. For example, a time-delay material such as glyceryl monostearate or glyceryl distearate may be employed. They may also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylenevinylacetate, methylcellulose, carboxymethylcellulose, protamine sulfate, or lactide/glycolide copolymers, polylactide/glycolide copolymers, or ethylenevinylacetate copolymers in order to control delivery of an administered composition. For example, the oral agent can be entrapped in microcapsules prepared by coacervation techniques or by interfacial polymerization, by the use of hydroxymethylcellulose or gelatin-microcapsules or poly (methylmethacrolate) microcapsules, respectively, or in a colloid drug delivery system. Colloidal dispersion systems include macromolecule complexes, nano-capsules, microspheres, microbeads, and lipid-based systems, including oil-in-water emulsions, micelles, mixed micelles, and liposomes. Methods for the preparation of the above-mentioned formulations will be apparent to those skilled in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture thereof. Such excipients can be suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example, arachis oil, olive oil, sesame oil or coconut oil, or in a mineral oil such as liquid paraffin. The oily suspensions may contain a thickening agent, for example, beeswax, hard paraffin, or cetyl alcohol. Sweetening agents such as those set forth above, and flavoring agents may be added to provide a palatable oral preparation.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified herein.

The pharmaceutical compositions of the present disclosure may also be in the form of oil-in-water emulsions. The oily phase may be a vegetable oil, for example, olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

In some embodiments, the pharmaceutical compositions comprise a dispersion comprising a therapeutically effective amount of etrumadenant, or a pharmaceutically acceptable salt thereof, and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), and N-tris[Hydroxymethyl]methyl-3-aminopropanesulfonic acid (TAPS).

After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampoule, syringe, or autoinjector (similar to, e.g., an EpiPen®)), whereas a multi-use container (e.g., a multi-use vial) is provided in other embodiments.

Formulations can also include carriers to protect the composition against rapid degradation or elimination from the body, such as a controlled release formulation, including liposomes, hydrogels, prodrugs and microencapsulated delivery systems. For example, a time delay material such as glyceryl monostearate or glyceryl stearate alone, or in combination with a wax, may be employed. Any drug delivery apparatus may be used to deliver the dispersions or compositions described herein, including implants (e.g., implantable pumps) and catheter systems, slow injection pumps and devices, all of which are well known to the skilled artisan.

Depot injections, which are generally administered subcutaneously or intramuscularly, may also be utilized to release the dispersions or compositions disclosed herein over a defined period of time. Depot injections are usually either solid- or oil-based and generally comprise at least one of the formulation components set forth herein. One of ordinary skill in the art is familiar with possible formulations and uses of depot injections.

The pharmaceutical compositions may be in the form of a sterile injectable aqueous or oleaginous suspension. This suspension may be formulated according to the known art using those suitable dispersing or wetting agents and suspending agents mentioned herein. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, Cremophor EL™ (BASF, Parsippany, NJ) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose any bland fixed oil may be employed, including synthetic mono- or diglycerides. Moreover, fatty acids such as oleic acid, find use in the preparation of injectables. Prolonged absorption of particular injectable formulations can be achieved by including an agent that delays absorption (e.g., aluminum monostearate or gelatin).

The present disclosure contemplates the administration of the dispersions or compositions described herein in the form of suppositories for rectal administration. The suppositories can be prepared by mixing the drug with a suitable non-irritating excipient which is solid at ordinary temperatures but liquid at the rectal temperature and will therefore melt in the rectum to release the drug. Such materials include, but are not limited to, cocoa butter and polyethylene glycols.

The dispersions or compositions described herein may be in the form of any other suitable pharmaceutical composition (e.g., sprays for nasal or inhalation use) currently known or developed in the future.

Some embodiments provide for a dosage form comprising a semi-solid dispersion as described herein. In some embodiments, the dosage form is a tablet or capsule. In some embodiments, the dosage form is a tablet. In some embodiments, the dosage form is a capsule. Some embodiments provide for a capsule comprising a semi-solid dispersion as described herein.

Some embodiments provide for a dosage form comprising a solid dispersion as described herein. Some embodiments provide for a tablet comprising a solid dispersion as described herein.

Some embodiments provide for a dosage form comprising spray dried particles as described herein.

In some embodiments, the dosage form is a tablet or capsule. In some embodiments, the dosage form is a tablet. In some embodiments, the dosage form is a capsule. Exemplary tablets and capsules are as described herein.

### Exemplary Compositions

Some embodiments provide for composition comprising a solid dispersion as described herein and one or more fillers, one or more disintegrants, optionally one or more glidants, and optionally one or more lubricants.

In some embodiments, the composition comprises about 40% to about 60% (w/w) of the solid dispersion. In some embodiments, the composition comprises about 45% to about 50% (w/w) of the solid dispersion.

In some embodiments, the composition comprises about 47% (w/w) of the solid dispersion, about 43% to about 47% (w/w) of the one or more fillers, about 4% to about 6% (w/w) of the one or more disintegrants, about 0.5 to about 1% (w/w) of the one or more glidants, and less than 1% (w/w) of the one or more lubricants.

In some embodiments, the composition comprises about 47.3% (w/w) of the solid dispersion, about 47.3% (w/w) of the one or more fillers, about 4.1% (w/w) of the one or more disintegrants, about 0.6% (w/w) of the one or more glidants, and about 0.6% (w/w) of the one or more lubricants.

In some embodiments, the composition comprises about 50% (w/w) of the solid dispersion, about 17.5% (w/w) of the one or more fillers, about 31.8% (w/w) of the one or more disintegrants, and about 0.6% (w/w) of the one or more lubricants.

In some embodiments, the composition comprises about 35% to about 45% (w/w) of the solid dispersion, about 40% to about 50% (w/w) of the one or more fillers, about 2% to about 7% (w/w) of the one or more disintegrants, and about 0.25% to about 0.75% (w/w) of the one or more lubricants.

In some embodiments, the composition comprises about 40% (w/w) of the solid dispersion, about 40% k to about 50% (w/w) of the one or more fillers, about 2% to about 7% (w/w) of the one or more disintegrants, and about 0.25% to about 0.75% (w/w) of the one or more lubricants.

In some embodiments, the composition comprises about 40% (w/w) of the solid dispersion, about 44% (w/w) of the one or more fillers, about 5% (w/w) of the one or more disintegrants, and about 0.5% (w/w) of the one or more lubricants.

In some embodiments, the one or more fillers is selected from anhydrous calcium hydrogen phosphate, calcium hydrogen phosphate dihydrate, tribasic calcium phosphate, guar gum, lactose monohydrate, mannitol, sorbitol, microcrystalline cellulose (MCC), co-processed MCC, silicified microcrystalline cellulose, and mesoporous silica;
the one or more disintegrants is croscarmellose sodium;
the one or more lubricants, if present, is sodium stearyl fumarate or magnesium stearate; and
the one or more glidant, if present, is colloidal silica.

In some embodiments, the one or more fillers is selected from anhydrous calcium hydrogen phosphate, calcium hydrogen phosphate dihydrate, tribasic calcium phosphate, guar gum, lactose monohydrate, mannitol, sorbitol, microcrystalline cellulose (MCC), co-processed MCC, silicified microcrystalline cellulose, and mesoporous silica; or
the one or more disintegrants is croscarmellose sodium; or
the one or more lubricants, if present, is sodium stearyl fumarate or magnesium stearate; or
the one or more glidant, if present, is colloidal silica.

In some embodiments, co-processed MCC comprises MCC co-processed with alginic acid, chitosan, DCP, guar gum, mannitol, silicon dioxide, sorbitol, and the like.

In some embodiments, the one or more fillers is selected from MCC, mannitol, silicified MCC, and mesoporous silica;
the one or more disintegrants is croscarmellose sodium;
the one or more lubricants, if present, is sodium stearyl fumarate or magnesium stearate; and
the one or more glidants, if present, is colloidal silica.

In some embodiments, the one or more fillers is selected from MCC, mannitol, silicified MCC, and mesoporous silica; or
the one or more disintegrants is croscarmellose sodium; or
the one or more lubricants, if present, is sodium stearyl fumarate or magnesium stearate; or
the one or more glidants, if present, is colloidal silica.

In some embodiments, the one or more fillers is selected from MCC, mannitol, silicified MCC, and mesoporous silica;
the one or more disintegrants is croscarmellose sodium; and
the one or more lubricants is magnesium stearate.

In some embodiments, the one or more fillers is selected from MCC, mannitol, silicified MCC, and mesoporous silica; or
the one or more disintegrants is croscarmellose sodium; or
the one or more lubricants is magnesium stearate.

In some embodiments, the composition as described herein comprises a first filler and a second filler, and wherein the solid dispersion, first filler, and second filler have a 2:1:1 weight ratio. In some embodiments, the composition as described herein comprises a first filler and a second filler, and wherein the solid dispersion, first filler, and second filler have about a 2.2:1.4:1 weight ratio.

In some embodiments, the first filler and the second filler are each independently selected from: anhydrous calcium hydrogen phosphate, calcium hydrogen phosphate dihydrate, tribasic calcium phosphate, guar gum, lactose monohydrate, mannitol, sorbitol, microcrystalline cellulose (MCC), co-processed MCC, silicified microcrystalline cellulose, and mesoporous silica.

In some embodiments, the first filler and the second filler are each independently selected from MCC, mannitol, silicified MCC, and mesoporous silica.

In some embodiments, the first filler is MCC, and the second filler is mannitol.

In some embodiments, the one or more disintegrants is croscarmellose sodium; the one or more lubricants is sodium stearyl fumarate; and the one or more glidants is colloidal silica.

In some embodiments, the one or more disintegrants is croscarmellose sodium; or the one or more lubricants is sodium stearyl fumarate; or the one or more glidants is colloidal silica.

In some embodiments, the one or more disintegrants is croscarmellose sodium; or the one or more lubricants is magnesium stearate.

In some embodiments, the composition comprises about 40% to about 60% (w/w) of the solid dispersion and one or more fillers, one or more disintegrants, optionally one or more glidants, and optionally one or more lubricants; wherein:
the one or more fillers is selected from anhydrous calcium hydrogen phosphate, calcium hydrogen phosphate dihydrate, tribasic calcium phosphate, guar gum, lactose monohydrate, mannitol, sorbitol, microcrystalline cellulose (MCC), co-processed MCC, silicified microcrystalline cellulose, and mesoporous silica;
the one or more disintegrants is croscarmellose sodium;
the one or more lubricants, if present, is sodium stearyl fumarate or magnesium stearate; and
the one or more glidant, if present, is colloidal silica.

In some embodiments, the composition comprises about 40% N to about 50% (w/w) of the solid dispersion and one or more fillers, one or more disintegrants, optionally one or more glidants, and optionally one or more lubricants; wherein:
the one or more fillers is selected from anhydrous calcium hydrogen phosphate, calcium hydrogen phosphate dihydrate, tribasic calcium phosphate, guar gum, lactose monohydrate, mannitol, sorbitol, microcrystalline cellulose (MCC), co-processed MCC, silicified microcrystalline cellulose, and mesoporous silica;
the one or more disintegrants is croscarmellose sodium;
the one or more lubricants, if present, is sodium stearyl fumarate or magnesium stearate; and
the one or more glidant, if present, is colloidal silica.

In some embodiments, the composition comprises about 40% to about 60% (w/w) of the solid dispersion and one or more fillers, one or more disintegrants, optionally one or more glidants, and optionally one or more lubricants; wherein:
the solid dispersion comprises about 25% to about 40% (w/w) of etrumadenant and about 60% to about 75% (w/w) of HPMCAS;
the one or more fillers is selected from anhydrous calcium hydrogen phosphate, calcium hydrogen phosphate dihydrate, tribasic calcium phosphate, guar gum, lactose monohydrate, mannitol, sorbitol, microcrystalline cellulose (MCC), co-processed MCC, silicified microcrystalline cellulose, and mesoporous silica;
the one or more disintegrants is croscarmellose sodium;
the one or more lubricants, if present, is sodium stearyl fumarate or magnesium stearate; and
the one or more glidant, if present, is colloidal silica.

In some embodiments, the composition comprises about 40% N to about 50% (w/w) of the solid dispersion and one or more fillers, one or more disintegrants, optionally one or more glidants, and optionally one or more lubricants; wherein:
the solid dispersion comprises about 25% to about 40% (w/w) of etrumadenant and about 60% to about 75% (w/w) of HPMCAS;
the one or more fillers is selected from anhydrous calcium hydrogen phosphate, calcium hydrogen phosphate dihydrate, tribasic calcium phosphate, guar gum, lactose monohydrate, mannitol, sorbitol, microcrystalline cellulose (MCC), co-processed MCC, silicified microcrystalline cellulose, and mesoporous silica;
the one or more disintegrants is croscarmellose sodium;
the one or more lubricants, if present, is sodium stearyl fumarate or magnesium stearate; and
the one or more glidant, if present, is colloidal silica.

In some embodiments, the composition comprises about 47% (w/w) of the solid dispersion; about 43% to about 47% (w/w) of the one or more fillers, about 4% to about 6% (w/w) of croscarmellose sodium, about 0.5 to about 1% (w/w) of colloidal silica, and less than 1% (w/w) of sodium stearyl fumarate; wherein:
the one or more fillers is selected from anhydrous calcium hydrogen phosphate, calcium hydrogen phosphate dihydrate, tribasic calcium phosphate, guar gum, lactose monohydrate, mannitol, sorbitol, microcrystalline cellulose (MCC), co-processed MCC, silicified microcrystalline cellulose, and mesoporous silica.

In some embodiments, the composition comprises about 47.3% (w/w) of the solid dispersion, about 47.3% (w/w) of the one or more fillers, about 4.1% (w/w) of croscarmellose sodium, about 0.6% (w/w) of colloidal silica, and about 0.6% (w/w) of sodium stearyl fumarate; wherein:
the one or more fillers is selected from anhydrous calcium hydrogen phosphate, calcium hydrogen phosphate dihydrate, tribasic calcium phosphate, guar gum, lactose monohydrate, mannitol, sorbitol, microcrystalline cellulose (MCC), co-processed MCC, silicified microcrystalline cellulose, and mesoporous silica.

In some embodiments, the composition comprises about 40% (w/w) of the solid dispersion; about 40% to about 50% (w/w) of the one or more fillers, about 2% to about 7% (w/w) of croscarmellose sodium, and about 0.25% to about 0.75% (w/w) of magnesium stearate; wherein:
the one or more fillers is selected from anhydrous calcium hydrogen phosphate, calcium hydrogen phosphate dihydrate, tribasic calcium phosphate, guar gum, lactose monohydrate, mannitol, sorbitol, microcrystalline cellulose (MCC), co-processed MCC, silicified microcrystalline cellulose, and mesoporous silica.

In some embodiments, the composition comprises about 40% (w/w) of the solid dispersion, about 44% (w/w) of the one or more fillers, about 5% (w/w) of croscarmellose sodium, about 0.5% (w/w) of magnesium stearate; wherein:
the one or more fillers is selected from anhydrous calcium hydrogen phosphate, calcium hydrogen phosphate dihydrate, tribasic calcium phosphate, guar gum, lactose monohydrate, mannitol, sorbitol, microcrystalline cellulose (MCC), co-processed MCC, silicified microcrystalline cellulose, and mesoporous silica.

In some embodiments, the composition comprises about 45% to about 50% (w/w) of the solid dispersion; about 43% to about 47% (w/w) of the one or more fillers, about 4% to about 6% (w/w) of croscarmellose sodium, about 0.5 to about 1% (w/w) of colloidal silica, and less than 1% (w/w) of sodium stearyl fumarate; wherein:
the one or more fillers is selected from MCC, mannitol, silicified MCC, and mesoporous silica.

In some embodiments, the composition comprises about 47% (w/w) of the solid dispersion; about 43% to about 47% (w/w) of the one or more fillers, about 4% to about 6% (w/w) of croscarmellose sodium, about 0.5 to about 1% (w/w) of colloidal silica, and less than 1% (w/w) of sodium stearyl fumarate; wherein:
the one or more fillers is selected from MCC, mannitol, silicified MCC, and mesoporous silica.

In some embodiments, the composition comprises about 47.3% (w/w) of the solid dispersion, about 47.3% (w/w) of the one or more fillers, about 4.1% (w/w) of croscarmellose sodium, about 0.6% (w/w) of colloidal silica, and about 0.6% (w/w) of sodium stearyl fumarate; wherein:
the one or more fillers is selected from MCC, mannitol, silicified MCC, and mesoporous silica.

In some embodiments, the composition comprises about 35% to about 45% (w/w) of the solid dispersion; about 40% to about 50% (w/w) of the one or more fillers, about 2% to about 7% (w/w) of croscarmellose sodium, and about 0.25% to about 0.75% (w/w) of magnesium stearate; wherein:
the one or more fillers is selected from MCC, mannitol, silicified MCC, and mesoporous silica.

In some embodiments, the composition comprises about 40% (w/w) of the solid dispersion; about 40% to about 50% (w/w) of the one or more fillers, about 2% to about 7% (w/w) of croscarmellose sodium, and about 0.25 to about 0.75% (w/w) of magnesium stearate; wherein:
the one or more fillers is selected from MCC, mannitol, silicified MCC, and mesoporous silica.

In some embodiments, the composition comprises about 40% (w/w) of the solid dispersion; about 44% (w/w) of the one or more fillers, about 5% (w/w) of croscarmellose sodium, and about 0.5% (w/w) of magnesium stearate; wherein:
the one or more fillers is selected from MCC, mannitol, silicified MCC, and mesoporous silica.

In some embodiments, the composition comprises about 45% to about 50% (w/w) of the solid dispersion; about 43% to about 47% (w/w) of the one or more fillers, about 4% to about 6% (w/w) of croscarmellose sodium, about 0.5 to about 1% (w/w) of colloidal silica, and less than 1% (w/w) of sodium stearyl fumarate; wherein:
the solid dispersion comprises about 25% to about 40% (w/w) of etrumadenant and about 60% to about 75% (w/w) of HPMCAS; and
the one or more fillers is selected from MCC, mannitol, silicified MCC, and mesoporous silica.

In some embodiments, the composition comprises about 47% (w/w) of the solid dispersion; about 43% to about 47% (w/w) of the one or more fillers, about 4% to about 6% (w/w) of croscarmellose sodium, about 0.5 to about 1% (w/w) of colloidal silica, and less than 1% (w/w) of sodium stearyl fumarate; wherein:
the solid dispersion comprises about 25% to about 40% (w/w) of etrumadenant and about 60% to about 75% (w/w) of HPMCAS; and
the one or more fillers is selected from MCC, mannitol, silicified MCC, and mesoporous silica.

In some embodiments, the composition comprises about 47.3% (w/w) of the solid dispersion, about 47.3% (w/w) of the one or more fillers, about 4.1% (w/w) of croscarmellose sodium, about 0.6% (w/w) of colloidal silica, and about 0.6% (w/w) of sodium stearyl fumarate; wherein:
the solid dispersion comprises about 25% to about 40% (w/w) of etrumadenant and about 60% to about 75% (w/w) of HPMCAS; and
the one or more fillers is selected from MCC, mannitol, silicified MCC, and mesoporous silica.

In some embodiments, the composition comprises about 35% to about 45% (w/w) of the solid dispersion; about 40% to about 50% (w/w) of the one or more fillers, about 2% to about 7% (w/w) of croscarmellose sodium, and about 0.25% to about 0.75% (w/w) of magnesium stearate; wherein:
the solid dispersion comprises about 20% to about 40% (w/w) of etrumadenant and about 60% to about 80% (w/w) of HPMCAS; and
the one or more fillers is selected from MCC, mannitol, silicified MCC, and mesoporous silica.

In some embodiments, the composition comprises about 40% (w/w) of the solid dispersion; about 40% to about 50% (w/w) of the one or more fillers, about 2% to about 7% (w/w) of croscarmellose sodium, and about 0.25 to about 0.75% (w/w) of magnesium stearate; wherein:
the solid dispersion comprises about 20% to about 40% (w/w) of etrumadenant and about 60% to about 80% (w/w) of HPMCAS; and
the one or more fillers is selected from MCC, mannitol, silicified MCC, and mesoporous silica.

In some embodiments, the composition comprises about 40% h/(w/w) of the solid dispersion; about 44% (w/w) of the one or more fillers, about 5% (w/w) of croscarmellose sodium, and about 0.5% (w/w) of magnesium stearate; wherein:
the solid dispersion comprises about 20% to about 40% (w/w) of etrumadenant and about 60% to about 80% (w/w) of HPMCAS; and
the one or more fillers is selected from MCC, mannitol, silicified MCC, and mesoporous silica.

In some embodiments, the composition comprises about 47% (w/w) of the solid dispersion, about 30% to about 50% (w/w) of a first filler and a second filler, about 4% to about 6% (w/w) of the one or more disintegrants, about 0.5 to about 1% (w/w) of the one or more glidants, and less than 1% (w/w) of the one or more lubricants; and wherein the solid dispersion, first filler, and second filler have a 2:1:1 weight ratio.

In some embodiments, the composition comprises about 40% (w/w) of the solid dispersion, about 40% to about 50% (w/w) of a first filler and a second filler, about 2% to about 7% (w/w) of the one or more disintegrants, and about 0.25% to about 0.75% (w/w) of the one or more lubricants; and wherein the solid dispersion, first filler, and second filler have about a 2.2:1.4:1 weight ratio.

In some embodiments, the composition comprises about 47% (w/w) of the solid dispersion, about 30% to about 50% (w/w) of a first filler and a second filler, about 4% to about 6% (w/w) of the one or more disintegrants, about 0.5 to about 1% (w/w) of the one or more glidants, and less than 1% (w/w) of the one or more lubricants; and
wherein the solid dispersion, first filler, and second filler have a 2:1:1 weight ratio; and wherein the first filler and the second filler are each independently selected from: selected from anhydrous calcium hydrogen phosphate, calcium hydrogen phosphate dihydrate, tribasic calcium phosphate, guar gum, lactose monohydrate, mannitol, sorbitol, microcrystalline cellulose (MCC), co-processed MCC, silicified microcrystalline cellulose, and mesoporous silica.

In some embodiments, the composition comprises about 40% (w/w) of the solid dispersion, about 40% to about 50% (w/w) of a first filler and a second tiller, about 2% to about 7% (w/w) of the one or more disintegrants, and about 0.25% to about 0.75% (w/w) of the one or more lubricants; and wherein the solid dispersion, first filler, and second filler have about a 2.2:1.4:1 weight ratio; and wherein the first filler and the second filler are each independently selected from: selected from anhydrous calcium hydrogen phosphate, calcium hydrogen phosphate dihydrate, tribasic calcium phosphate, guar gum, lactose monohydrate, mannitol, sorbitol, microcrystalline cellulose (MCC), co-processed MCC, silicified microcrystalline cellulose, and mesoporous silica.

In some embodiments, the composition comprises about 47% (w/w) of the solid dispersion, about 30% to about 50% (w/w) of a first filler and a second filler, about 4% to about 6% (w/w) of the one or more disintegrants, about 0.5 to about 1% (w/w) of the one or more glidants, and less than 1% (w/w) of the one or more lubricants; and
wherein the solid dispersion, first filler, and second filler have a 2:1:1 weight ratio; and wherein the first filler and the second filler are each independently selected from MCC, mannitol, silicified MCC, and mesoporous silica.

In some embodiments, the composition comprises about 40% (w/w) of the solid dispersion, about 40% to about 50% (w/w) of a first filler and a second filler, about 2% to about 7% (w/w) of the one or more disintegrants, and about 0.25% to about 0.75% (w/w) of the one or more lubricants; and wherein the solid dispersion, first filler, and second filler have about a 2.2:1.4:1 weight ratio; and wherein the first filler and the second filler are each independently selected from MCC, mannitol, silicified MCC, and mesoporous silica.

In some embodiments, the composition comprises about 47% (w/w) of the solid dispersion, about 30% to about 50% (w/w) of a first filler and a second filler, about 4% to about 6% (w/w) of the one or more disintegrants, about 0.5 to about 1% (w/w) of the one or more glidants, and less than 1% (w/w) of the one or more lubricants; and
wherein the solid dispersion, first filler, and second filler have a 2:1:1 weight ratio; and wherein the first filler is MCC, and the second filler is mannitol.

In some embodiments, the composition comprises about 40% (w/w) of the solid dispersion, about 40% to about 50% (w/w) of a first filler and a second filler, about 2% to about 7% (w/w) of the one or more disintegrants, and about 0.25% to about 0.75% (w/w) of the one or more lubricants; and wherein the solid dispersion, first filler, and second filler have about a 2.2:1.4:1 weight ratio; and wherein the first filler is MCC, and the second filler is mannitol.

In some embodiments, the composition comprises about 479 (w/w) of the solid dispersion, about 30% to about 50% (w/w) of a first filler and a second filler, about 4% to about 6% (w/w) of croscarmellose sodium, about 0.5 to about 1% (w/w) of colloidal silica, and less than 1% (w/w) of sodium stearyl fumarate; and
wherein the solid dispersion, first filler, and second filler have a 2:1:1 weight ratio; and wherein the first filler is MCC, and the second filler is mannitol.

In some embodiments, the composition comprises about 40% (w/w) of the solid dispersion, about 40% to about 50% (w/w) of a first filler and a second filler, about 2% to about 7% (w/w) of croscarmellose sodium, and about 0.25% to about 0.75% (w/w) of magnesium stearate; and wherein the solid dispersion, first filler, and second filler have about a 2.2:1.4:1 weight ratio; and wherein the first filler is MCC, and the second filler is mannitol.

Some embodiments provide for an intragranular component comprising a composition as described herein.

In some embodiments, an intragranular component comprises a solid dispersion as described herein and one or more fillers, one or more disintegrants, optionally one or more glidants, and optionally one or more lubricants.

### Granules

Some embodiments provide for a granule comprising a solid dispersion as described herein and one or more fillers, one or more disintegrants, optionally one or more glidants, and optionally one or more lubricants.

Some embodiments provide for a granule comprising spray dried particles as described herein and one or more fillers, one or more disintegrants, optionally one or more glidants, and optionally one or more lubricants.

In some embodiments, the granule comprises about 40% to about 60% (w/w) of the solid dispersion or spray-dried particles. In some embodiments, the granule comprises about 45% to about 50% (w/w) of the solid dispersion or spray-dried particles.

In some embodiments, the granule comprises: about 47% (w/w) of the solid dispersion or spray-dried particles, about 43% to about 47% (w/w) of the one or more fillers, about 4% to about 6% (w/w) of the one or more disintegrants, about 0.5 to about 1% (w/w) of the one or more glidants, and less than 1% (w/w) of the one or more lubricants.

In some embodiments, the granule comprises: about 47.3% (w/w) of the solid dispersion or spray-dried particles, about 47.3% (w/w) of the one or more fillers, about 4.1% (w/w) of the one or more disintegrants, about 0.6% (w/w) of the one or more glidants, and about 0.6% (w/w) of the one or more lubricants.

In some embodiments, the granule comprises: about 35% to about 45% (w/w) of the solid dispersion or spray-dried particles, about 40% to about 50% (w/w) of the one or more fillers, about 2% to about 7% (w/w) of the one or more disintegrants, and about 0.25% to about 0.75% (w/w) of the one or more lubricants.

In some embodiments, the granule comprises: about 40% (w/w) of the solid dispersion or spray-dried particles, about 44% (w/w) of the one or more fillers, about 5% (w/w) of the one or more disintegrants, and about 0.5% (w/w) of the one or more lubricants.

In some embodiments, the one or more fillers is selected from anhydrous calcium hydrogen phosphate, calcium hydrogen phosphate dihydrate, tribasic calcium phosphate, guar gum, lactose monohydrate, mannitol, sorbitol, microcrystalline cellulose (MCC), co-processed MCC, silicified microcrystalline cellulose, and mesoporous silica; and/or
the one or more disintegrants is croscarmellose sodium; and/or
the one or more lubricants, if present, is sodium stearyl fumarate or magnesium stearate; and/or
the one or more glidant, if present, is colloidal silica.

In some embodiments, the one or more fillers is selected from MCC, mannitol, silicified MCC, and mesoporous silica; and/or
the one or more disintegrants is croscarmellose sodium; and/or
the one or more lubricants, if present, is sodium stearyl fumarate or magnesium stearate; and/or
the one or more glidants, if present, is colloidal silica.

In some embodiments, the granule described herein comprises a first filler and a second filler, and wherein the solid dispersion, first filler, and second filler have a 2:1:1 weight ratio. In some embodiments, the granule described herein comprises a first filler and a second filler, and wherein the solid dispersion, first filler, and second filler have about a 2.2:1.4:1 weight ratio.

In some embodiments, the first filler is MCC and the second filler is mannitol.

In some embodiments, the one or more disintegrants is croscarmellose sodium; and/or
the one or more lubricants is sodium stearyl fumarate or magnesium stearate; and/or
the one or more glidants, if present, is colloidal silica.

In some embodiments, the one or more disintegrants is croscarmellose sodium; and/or the one or more lubricants is sodium stearyl fumarate or magnesium stearate; and/or the one or more glidants, if present, is selected from colloidal silica.

### Exemplary Pharmaceutical Compositions

Some embodiments provide for a pharmaceutical composition comprising an intragranular component comprising a composition as described herein, and an extragranular component comprising one or more fillers, optionally one or more disintegrants, optionally one or more glidants, and one or more lubricants.

Some embodiments provide for a pharmaceutical composition comprising an intragranular component as described herein, and an extragranular component comprising one or more fillers, optionally one or more disintegrants, optionally one or more glidants, and one or more lubricants.

In some embodiments, the pharmaceutical composition comprises about 85% to about 95% (w/w) of the intragranular component, or about 85% (w/w) of the intragranular component. In some embodiments, the pharmaceutical composition comprises about 85% to about 95% (w/w) of the intragranular component. In some embodiments, the pharmaceutical composition comprises about 87% c to about 93% (w/w) of the intragranular component. In some embodiments, the pharmaceutical composition comprises about 85% (w/w) of the intragranular component. In some embodiments, the pharmaceutical composition comprises about 90% (w/w) of the intragranular component.

In some embodiments, the extragranular component comprises: about 10% to about 15% (w/w) of the one or more fillers, about 1% to about 2% (w/w) of the one or more disintegrants, about 0.5% to about 1.0% (w/w) of the one or more glidants, and about 0.5% to about 1.0% (w/w) of the one or more lubricants. In some embodiments, the extragranular component comprises: about 5% to about 15% (w/w) of the one or more fillers and about 0.25% to about 0.75% (w/w) of the one or more lubricants.

In some embodiments, the one or more fillers is selected from anhydrous calcium hydrogen phosphate, calcium hydrogen phosphate dihydrate, tribasic calcium phosphate, guar gum, lactose monohydrate, mannitol, sorbitol, microcrystalline cellulose, co-processed microcrystalline cellulose, silicified microcrystalline cellulose, and mesoporous silica;
the one or more disintegrants, if present, is croscarmellose sodium;
the one or more lubricants is sodium stearyl fumarate or magnesium stearate; and
the one or more glidants, if present, is colloidal silica.

In some embodiments, the one or more fillers is selected from anhydrous calcium hydrogen phosphate, calcium hydrogen phosphate dihydrate, tribasic calcium phosphate, guar gum, lactose monohydrate, mannitol, sorbitol, microcrystalline cellulose, co-processed microcrystalline cellulose, silicified microcrystalline cellulose, and mesoporous silica; or
the one or more disintegrants, if present, is croscarmellose sodium; or
the one or more lubricants is sodium stearyl fumarate or magnesium stearate; or
the one or more glidants, if present, is colloidal silica.

In some embodiments, the one or more fillers is selected from microcrystalline cellulose, mannitol, silicified microcrystalline cellulose, and mesoporous silica;
the one or more disintegrants is croscarmellose sodium;
the one or more lubricants is sodium stearyl fumarate; and
the one or more glidants is colloidal silica.

In some embodiments, the one or more fillers is selected from microcrystalline cellulose, mannitol, silicified microcrystalline cellulose, and mesoporous silica; or
the one or more disintegrants is croscarmellose sodium; or
the one or more lubricants is sodium stearyl fumarate; or
the one or more glidants is colloidal silica.

In some embodiments, the one or more fillers is selected from microcrystalline cellulose and mannitol, and the one or more lubricants is magnesium stearate.

In some embodiments, the pharmaceutical composition comprises two fillers, optionally in a 1:1 to a 2:1 ratio.

In some embodiments, the pharmaceutical composition comprises about 85% to about 95% (w/w) of the intragranular component, and an extragranular component; wherein:
the intragranular component comprises:
   about 35% to about 50% (w/w) of the solid dispersion and one or more fillers, one or more disintegrants, optionally one or more glidants, and optionally one or more lubricants; and
the extragranular component comprises:
   about 5% to about 15% (w/w) of the one or more fillers, optionally about 1% to about 2% (w/w) of the one or more disintegrants, optionally about 0.5% to about 1.0% (w/w) of the one or more glidants, and about 0.25% to about 1.0% (w/w) of the one or more lubricants:
wherein:
   the one or more fillers is selected from anhydrous calcium hydrogen phosphate, calcium hydrogen phosphate dihydrate, tribasic calcium phosphate, guar gum, lactose monohydrate, mannitol, sorbitol, microcrystalline cellulose (MCC), co-processed MCC, silicified microcrystalline cellulose, and mesoporous silica;
   the one or more disintegrants is croscarmellose sodium;
   the one or more lubricants, if present, is sodium stearyl fumarate or magnesium stearate; and
   the one or more glidant, if present, is colloidal silica.

In some embodiments, the pharmaceutical composition comprises about 85% to about 95% (w/w) of the intragranular component, and an extragranular component; wherein:
the intragranular component comprises:
   about 35% to about 50% (w/w) of the solid dispersion and one or more fillers, one or more disintegrants, optionally one or more glidants, and optionally one or more lubricants; and
the extragranular component comprises:
   about 5% to about 15% (w/w) of the one or more fillers, optionally about 1% to about 2% (w/w) of the one or more disintegrants, optionally about 0.5% to about 1.0% (w/w) of the one or more glidants, and about 0.25% to about 1.0% (w/w) of the one or more lubricants;
wherein:
   the one or more fillers is selected from microcrystalline cellulose, mannitol, silicified microcrystalline cellulose, and mesoporous silica;
   the one or more disintegrants is croscarmellose sodium;
   the one or more lubricants, if present, is sodium stearyl fumarate or magnesium stearate; and
   the one or more glidant, if present, is colloidal silica.

In some embodiments, the pharmaceutical composition comprises about 85% to about 95% (w/w) of the intragranular component, and an extragranular component; wherein:
the intragranular component comprises:
   about 35% to about 50% (w/w) of the solid dispersion and one or more tillers, one or more disintegrants, optionally one or more glidants, and optionally one or more lubricants,
   wherein the solid dispersion comprises about 20% to about 40% (w/w) of etrumadenant and about 60% to about 80% (w/w) of HPMCAS; and
the extragranular component comprises:
   about 5% to about 15% (w/w) of the one or more fillers, optionally about 1% to about 2% (w/w) of the one or more disintegrants, optionally about 0.5% to about 1.0% (w/w) of the one or more glidants, and about 0.25% to about 1.0% (w/w) of the one or more lubricants:
wherein:
   the one or more fillers is selected from anhydrous calcium hydrogen phosphate, calcium hydrogen phosphate dihydrate, tribasic calcium phosphate, guar gum, lactose monohydrate, mannitol, sorbitol, microcrystalline cellulose (MCC), co-processed MCC, silicified microcrystalline cellulose, and mesoporous silica;
   the one or more disintegrants is croscarmellose sodium;
   the one or more lubricants, if present, is sodium stearyl fumarate or magnesium stearate; and
   the one or more glidant, if present, is colloidal silica.

In some embodiments, the pharmaceutical composition comprises about 85% to about 95% (w/w) of the intragranular component, and an extragranular component; wherein:
the intragranular component comprises:
   about 45% to about 50% (w/w) of the solid dispersion and one or more tillers, one or more disintegrants, optionally one or more glidants, and optionally one or more lubricants,
   wherein the solid dispersion comprises about 25% to about 40% (w/w) of etrumadenant and about 60% to about 75% (w/w) of HPMCAS; and
the extragranular component comprises:
   about 10% to about 15% (w/w) of the one or more fillers, about 1% to about 2% (w/w) of the one or more disintegrants, about 0.5% to about 1.0% (w/w) of the one or more glidants, and about 0.5% to about 1.0% (w/w) of the one or more lubricants;
wherein:
   the one or more fillers is selected from microcrystalline cellulose, mannitol, silicified microcrystalline cellulose, and mesoporous silica;
   the one or more disintegrants is croscarmellose sodium;
   the one or more lubricants, if present, is sodium stearyl fumarate; and
   the one or more glidant, if present, is colloidal silica.

In some embodiments, the pharmaceutical composition comprises about 85% to about 95% (w/w) of the intragranular component, and an extragranular component; wherein:
the intragranular component comprises:
   about 35% to about 45% (w/w) of the solid dispersion and one or more fillers, one or more disintegrants, optionally one or more glidants, and optionally one or more lubricants,
   wherein the solid dispersion comprises about 20% to about 40% (w/w) of etrumadenant and about 60% to about 80% (w/w) of HPMCAS; and
the extragranular component comprises:
   about 5% to about 15% (w/w) of the one or more fillers and about 0.25% to about 0.75% (w/w) of the one or more lubricants;
   wherein the one or more fillers is selected from microcrystalline cellulose and mannitol; the one or more disintegrants is croscarmellose sodium; and the one or more lubricants is magnesium stearate.

In some embodiments, the pharmaceutical composition comprises about 85% to about 95% (w/w) of the intragranular component, and an extragranular component; wherein:
the intragranular component comprises about 47% (w/w) of the solid dispersion; about 43% to about 47% (w/w) of the one or more fillers, about 4% to about 6% (w/w) of croscarmellose sodium, about 0.5 to about 1% (w/w) of colloidal silica, and less than 1% (w/w) of sodium stearyl fumarate; and
the extragranular component comprises: about 10% to about 15% (w/w) of the one or more fillers, about 1% to about 2% (w/w) of croscarmellose sodium, about 0.5% to about 1.0% (w/w) of colloidal silica, and about 0.5% to about 1.0% (w/w) of sodium stearyl fumarate;
wherein the one or more fillers is selected from anhydrous calcium hydrogen phosphate, calcium hydrogen phosphate dihydrate, tribasic calcium phosphate, guar gum, lactose monohydrate, mannitol, sorbitol, microcrystalline cellulose (MCC), co-processed MCC, silicified microcrystalline cellulose, and mesoporous silica.

In some embodiments, the pharmaceutical composition comprises about 85% to about 95% (w/w) of the intragranular component, and an extragranular component; wherein:
the intragranular component comprises about 35% to about 45% (w/w) of the solid dispersion; about 40% to about 50% (w/w) of the one or more fillers, about 2% to about 7% (w/w) of croscarmellose sodium, and about 0.25% to about 0.75% (w/w) of magnesium stearate; and
the extragranular component comprises: about 5% to about 15% (w/w) of the one or more fillers and about 0.25% to about 0.75% (w/w) of the one or more lubricants;
wherein the one or more fillers is selected from anhydrous calcium hydrogen phosphate, calcium hydrogen phosphate dihydrate, tribasic calcium phosphate, guar gum, lactose monohydrate, mannitol, sorbitol, microcrystalline cellulose (MCC), co-processed MCC, silicified microcrystalline cellulose, and mesoporous silica.

In some embodiments, the pharmaceutical composition comprises about 85% to about 95% (w/w) of the intragranular component, and an extragranular component; wherein:
the intragranular component comprises about 47.3% (w/w) of the solid dispersion, about 47.3% (w/w) of the one or more tillers, about 4.1% (w/w) of croscarmellose sodium, about 0.6% (w/w) of colloidal silica, and about 0.6% (w/w) of sodium stearyl fumarate; and
the extragranular component comprises: about 10% to about 15% (w/w) of the one or more fillers, about 1% to about 2% (w/w) of croscarmellose sodium, about 0.5% to about 1.0% (w/w) of colloidal silica, and about 0.5% to about 1.0% (w/w) of sodium stearyl fumarate;
wherein the one or more tillers is selected from anhydrous calcium hydrogen phosphate, calcium hydrogen phosphate dihydrate, tribasic calcium phosphate, guar gum, lactose monohydrate, mannitol, sorbitol, microcrystalline cellulose (MCC), co-processed MCC, silicified microcrystalline cellulose, and mesoporous silica.

In some embodiments, the pharmaceutical composition comprises about 85% to about 95% (w/w) of the intragranular component, and an extragranular component; wherein:
the intragranular component comprises about 47% (w/w) of the solid dispersion; about 43% to about 47% (w/w) of the one or more fillers, about 4% to about 6% (w/w) of croscarmellose sodium, about 0.5 to about 1% (w/w) of colloidal silica, and less than 1% (w/w) of sodium stearyl fumarate; and
the extragranular component comprises: about 10% to about 15% (w/w) of the one or more fillers, about 1% to about 2% (w/w) of croscarmellose sodium, about 0.5% to about 1.0% (w/w) of colloidal silica, and about 0.5% to about 1.0% (w/w) of sodium stearyl fumarate;
wherein the one or more fillers is selected from microcrystalline cellulose, mannitol, silicified microcrystalline cellulose, and mesoporous silica.

In some embodiments, the pharmaceutical composition comprises about 85% to about 95% (w/w) of the intragranular component, and an extragranular component; wherein:
the intragranular component comprises about 47.3% (w/w) of the solid dispersion, about 47.3% (w/w) of the one or more fillers, about 4.1% (w/w) of croscarmellose sodium, about 0.6% (w/w) of colloidal silica, and about 0.6% (w/w) of sodium stearyl fumarate; and
the extragranular component comprises: about 10% to about 15% (w/w) of the one or more fillers, about 1% to about 2% (w/w) of croscarmellose sodium, about 0.5% to about 1.0% (w/w) of colloidal silica, and about 0.5% to about 1.0% (w/w) of sodium stearyl fumarate;
wherein the one or more fillers is selected from microcrystalline cellulose, mannitol, silicified microcrystalline cellulose, and mesoporous silica.

In some embodiments, the pharmaceutical composition comprises about 85% to about 95% (w/w) of the intragranular component, and an extragranular component; wherein:
the intragranular component comprises about 35% to about 45% (w/w) of the solid dispersion; about 40% to about 50% (w/w) of the one or more fillers, about 2% to about 7% (w/w) of croscarmellose sodium, and about 0.25% to about 0.75% (w/w) of magnesium stearate; and
the extragranular component comprises: about 5% to about 15% (w/w) of the one or more fillers and about 0.25% to about 0.75% (w/w) of magnesium stearate;
wherein the one or more tillers is selected from microcrystalline cellulose and mannitol.

In some embodiments, the one or more fillers of the intragranular component and the one or more fillers of the extragranular component are each independently selected from the fillers described above. In some embodiments, the one or more fillers of the intragranular component are the same as the one or more fillers of the extragranular component. In some embodiments, the one or more fillers of the intragranular component are different than the one or more fillers of the extragranular component.

In some embodiments, the pharmaceutical composition or composition is a formulation of Table 16. Table 19, Table 20, or Table 24.

### Exemplary Tablets

Some embodiments provide for a pharmaceutical composition or a composition as described herein formulated as a tablet. In some embodiments, tablets described herein may be made according to methods known in the art.

Some embodiments provide for tablets that are physically stable, are chemically stable, have stable in vitro dissolution properties, and/or have stable in vivo properties upon storage at 25° C. and 60% relative humidity (long term stability testing) or 40° C. and 75% relative humidity (accelerated stability testing) in a closed or open packaging for 3 months or more (e.g., 3, 6, 9, 12, 15, 18, 21, 24, 27, 30, 33, 36 months). Stability may be evaluated by a variety of analytical methods including assay of etrumadenant and impurities by high-performance liquid chromatography (HPLC), non-sink dissolution testing, sink dissolution testing, and pharmacokinetics analysis under fed and/or fasted conditions. Some embodiments provide for tablets that are physically stable, are chemically stable, have stable in vitro dissolution properties, and/or have stable in vivo properties upon storage at 25° C. and 60% relative humidity (long term stability testing) or 40° C. and 75% relative humidity (accelerated stability testing) in a closed or open packaging for 6 months, 9 months, or 12 months.

Some embodiments provide for a tablet comprising microcrystalline cellulose (MCC), mannitol, croscarmellose sodium, colloidal silica, sodium stearyl fumarate, and a solid dispersion as described herein. Some embodiments provide for a tablet comprising microcrystalline cellulose (MCC), mannitol, croscarmellose sodium, magnesium stearate, and a solid dispersion as described herein.

In some embodiments, the tablet comprises about 25% to about 60% (w/w) of the solid dispersion. In some embodiments, the tablet comprises about 35% to about 55% (w/w) of the solid dispersion. In some embodiments, the tablet comprises about 30% to about 55% (w/w) of the solid dispersion. In some embodiments, the tablet comprises about 35% to about 45% (w/w) of the solid dispersion. In some embodiments, the tablet comprises about 40% to about 50% (w/w) of the solid dispersion.

In some embodiments, the tablet comprises about 40% to about 50% (w/w) of the solid dispersion as described herein, about 10% to 25% (w/w) of MCC, about 20% to about 25% (w/w) of mannitol, about 5% to about 6% (w/w) of croscarmellose sodium, about 1% (w/w) of colloidal silica, and about 1% (w/w) of sodium stearyl fumarate. In some embodiments, the tablet comprises about 35% to about 45% (w/w) of the solid dispersion as described herein, about 30% to about 42% (w/w) of MCC, about 13% to about 23% (w/w) of mannitol, about 2% to about 8% (w/w) croscarmellose sodium, and about 0.5% to about 1.5% (w/w) magnesium stearate.

In some embodiments, the tablet is a tablet of Table 16, Table 19. Table 20, or Table 24.

In some embodiments, the tablet has a weight of about 100 mg to about 1 g. In some embodiments, the tablet has a weight of about 100 mg, about 250 mg, about 500 mg, or about 750 mg. In some embodiments, the tablet has a weight of 750 mg to 800 mg, 760 mg to 790 mg, 770 mg to 780 mg, 775 mg to 785 mg, or 780 mg to 790 mg.

In some embodiments, the tablet has a weight of 1 g. In some embodiments, the tablet has a weight of about 100 mg. In some embodiments, the tablet has a weight of about 250 mg. In some embodiments, the tablet has a weight of about 500 mg. In some embodiments, the tablet has a weight of about 750 mg.

In some embodiments, tablets described herein further comprise a coating. In some embodiments, the coating is a non-functional coating.

Some embodiments provide for tablets as described herein, wherein the tablet is an immediate release tablet. Some embodiments provide for tablets as described herein, wherein the percent of etrumadenant released at 45 minutes is not less than 85% as measured by the dissolution method of Table 21.

Some embodiments provide for tablets as described herein, wherein the tablet has a total impurity amount of less than 2% (% area), measured by HPLC. Analysis of impurity by HPLC may be carried out as described herein. In some embodiments, the tablet has a total impurity amount of about 1.9%, about 1.8%, about 1.7%, about 1.6%, about 1.5%, about 1.4%, about 1.3%, about 1.2%, about 1.1%, about 1.0%, about 0.9%, about 0.8%, about 0.7%, about 0.6%, about 0.5%, about 0.4%, about 0.3%, about 0.2%, about 0.1% (% area), measured by HPLC. In some embodiments, the tablet has a total impurity amount of less than 1% (% area), measured by HPLC. In some embodiments, the tablet has a total impurity amount of less than 0.5% (% area), measured by HPLC.

Some embodiments provide for tablets as described herein, wherein, upon storage at 25° C. and 60% relative humidity in a closed packaging for 6 months, 12 months, 18 months, 24 months, or 36 months:
(i) the amount of etrumadenant in the tablet is about 90 to about 110% of the value measured at 0 months;
(ii) a total impurity amount of less than or equal to 2%, less than or equal to 1%, or less than or equal to 0.5% (% area), measured by HPLC;
(iii) the amount of etrumadenant measured at 210 minutes by non-sink dissolution testing (C₂₁₀(μgA/mL) or AUC_{35-210 FaSSIF} (min*μgA/mL)) differs by no more than 15% of the value measured at 0 months; or
(iv) any combination of (i) to (iii).

In some embodiments, upon storage at 25° C. and 60% relative humidity in a closed packaging for 6 months, 12 months, 18 months, 24 months, or 36 months:
(i) the amount of etrumadenant in the tablet is about 90 to about 110% of the value measured at 0 months;
(ii) a total impurity amount of less than or equal to 2%, less than or equal to 1%, or less than or equal to 0.5% (% area), measured by HPLC; or
(iii) any combination of (i) to (ii).

In some embodiments, the total impurity amount and the amount of etrumadenant at 210 minutes by non-sink dissolution is as described herein.

Tablets described herein may be administered to a human in a fed or fasted state. In some embodiments, upon administration of the tablet to a human in a fed or fasted state, the human's overall exposure to etrumadenant is equivalent. Overall exposure may be measured by a ratio of AUCₗₐₛₜ (fasted) to AUCₗₐₛₜ(fed) or by a ratio of AUC_{inf} (fasted) to AUC (fed). In some embodiments, equivalence refers to achieving equivalence limits of 80-125 percent for AUC_{0-inf}.

It is contemplated that the tablets described herein exhibit desirable properties, including but not limited to advantageous levels of weight, thickness, breaking force, flowability, tabletability and/or compressibility as well as robust disintegration profiles as further described herein.

Some embodiments provide for methods of making a tablet as described herein. In some embodiments, tablets described herein may be made according to a method as described in the Examples provided herein. In some embodiments, certain components of the tablet may be blended, lubricated, de-lumped, roller compacted, milled, and/or compressed. The order of such steps may be modified as needed. In some embodiments, a film-coating may be applied. In some embodiments, the resulting tablet and/or film-coated tablet may be packaged accordingly, for example, to form a kit as described herein.

Some embodiments provide for a process of making a tablet described herein, comprising: (i) blending a dispersion as described herein with one or more excipients as described herein, wherein the blending optionally may occur in one or more steps to provide a mixture; (ii) adding a lubricant; (iii) de-lumping; (iv) roller-compacting and milling to form granules; (v) adding one or more additional excipients to the granules to form a second mixture; (vi) compressing the second mixture to form a tablet; and optionally (vii) adding a film-coating to form a film-coated tablet. In some embodiments, the order of step (ii) (adding a lubricant) and step (iii) may be reversed such that de-lumping occurs prior to adding a lubricant.

Some embodiments provide for a process of making a tablet described herein, comprising: (i) blending a dispersion as described herein with one or more excipients as described herein to provide a mixture; (ii) milling/dispersing the mixture; (iii) adding a lubricant and blending to form a second mixture; (iv) compacting; (v) milling; (vi) adding one or more additional excipients and blending to form a third mixture; (vi) compressing the third mixture to form a tablet; and optionally (vii) adding a film-coating to form a film-coated tablet.

### V. Therapeutic and Prophylactic Uses

The present disclosure provides methods for using dispersions described herein, compositions described herein, granules described herein, pharmaceutical compositions described herein, dosage forms described herein, or tablets described herein, for inhibiting adenosine A_{2A} receptor (A_{2A}R), adenosine A_{2B} receptor (A_{2B}R), or adenosine A_{2A} receptor (A_{2B}R) and adenosine A_{2B} receptor (A_{2B}R).

As used herein, the terms “inhibit”, ‘inhibition” and the like refer to the ability of an antagonist to decrease the function or activity of a particular target, e.g., A_{2A}R, A_{2B}R, or both A_{2A}R and A_{2B}R. The decrease is preferably at least a 50% and may be, for example, at least about 55%, at least about 60%, at least about 65%, at least about 70%, at least about 75%, at least about 80%, at least about 85%, at least about 90%, or at least about 95%.

The present disclosure contemplates the administration of the dispersions, compositions, or pharmaceutical compositions described herein in any appropriate manner. Suitable routes of administration include oral, parenteral (e.g., intramuscular, intravenous, subcutaneous (e.g., injection or implant), intraperitoneal, intracisternal, intraarticular, intracerebral (intraparenchymal) and intracerebroventricular), nasal, vaginal, sublingual, intraocular, rectal, topical (e.g., transdermal), buccal and inhalation. Depot injections, which are generally administered subcutaneously or intramuscularly, may also be utilized to release the solid forms of the dispersion of etrumadenant disclosed herein over a defined period of time. Some embodiments of the present disclosure contemplate oral administration.

The present disclosure also encompasses the use of dispersions described herein, compositions described herein, and pharmaceutical compositions described herein for the treatment or prevention of diseases, disorders, and/or conditions that would benefit from inhibition of A_{2A}R, A_{2B}R, or both A_{2A}R and A_{2B}R. While particular uses are described in detail hereafter, it is to be understood that the present disclosure is not so limited. Furthermore, although general categories of particular diseases, disorders, and conditions are set forth hereafter, some of the diseases, disorders, and conditions may be a member of more than one category, and others may not be a member of any of the disclosed categories.

Some embodiments provide for a method of treating a disease, disorder, or condition, mediated at least in part by the adenosine A_{2A} receptor (A_{2A}R) and/or the adenosine A_{2B} receptor (A_{2B}R), comprising administering a dosage form as described herein, a pharmaceutical composition as described herein, or a tablet as described herein to a subject in need thereof

In some embodiments, the diseases, disorders, and/or conditions described herein are mediated, at least in part, by A_{2A}R. In some embodiments, the diseases, disorders, and/or conditions described herein are mediated, at least in part, by A_{2B}R. In some embodiments, the diseases, disorders, and/or conditions described herein are mediated, at least in part, by both A_{2A}R and A_{2B}R. In some embodiments, the dispersions, compositions, or pharmaceutical compositions described herein may be administered in an amount effective to treat or prevent cardiovascular diseases, CNS-related and neurological disorders, immune-related disorders, metabolic diseases, microbial-related disorders, or oncology and oncology-related diseases. Cardiovascular diseases, CNS-related and neurological disorders, immune-related disorders, metabolic diseases, microbial-related disorders, or oncology and oncology-related diseases for which treatment with an AAR, A_{2B}R, or both AAR and A_{2B}R inhibitor may be beneficial are described in WO2018136700 and WO2020018680A1, the disclosures of which are incorporated herein by reference.

In some embodiments, the dispersions, compositions, or pharmaceutical compositions described herein are administered in an amount effective to decrease or reverse the immunosuppression mediated by A_{2A}R, or A_{2B}R, or both A_{2A}R and A_{2B}R.

In some embodiments, the dispersions, compositions, or pharmaceutical compositions described herein are administered in an amount to increase or enhance an immune response, to improve immunization, including increasing vaccine efficacy, or to increase inflammation.

In some embodiments of the aforementioned methods, the dispersions described herein, compositions described herein, or pharmaceutical compositions described herein are used in combination with at least one additional therapy. Each additional therapy can be a therapeutic agent or another treatment modality. Additional therapies contemplated include those described in WO2018136700 and WO2020018680A1, as well as those descried below. In embodiments comprising one or more additional therapeutic agents, each agent may target a different, but complementary, mechanism of action. The additional therapeutic agents can be small chemical molecules; macromolecules such as proteins, antibodies, peptibodies, peptides, DNA, RNA or fragments of such macromolecules; or cellular or gene therapies. Non-limiting examples of additional treatment modalities include surgical resection of a tumor, bone marrow transplant, radiation therapy, and photodynamic therapy. The use of dispersions, compositions or pharmaceutical compositions described herein in combination with one or more additional therapies may have a synergistic therapeutic or prophylactic effect on the underlying disease, disorder, or condition. In addition or alternatively, the combination therapy may allow for a dose reduction of one or more of the therapies, thereby ameliorating, reducing or eliminating adverse effects associated with one or more of the agents.

In embodiments comprising one or more additional treatment modality, the dispersions, compositions or pharmaceutical compositions described herein can be administered before, after or during treatment with the additional treatment modality. In embodiments comprising one or more additional therapeutic agent, the therapeutic agents used in such combination therapy can be formulated as a single composition or as separate compositions. If administered separately, each therapeutic agent in the combination can be given at or around the same time, or at different times. Furthermore, the therapeutic agents are administered “in combination” even if they have different forms of administration (e.g., oral capsule and intravenous), they are given at different dosing intervals, one therapeutic agent is given at a constant dosing regimen while another is titrated up, titrated down or discontinued, or each therapeutic agent in the combination is independently titrated up, titrated down, increased or decreased in dosage, or discontinued and/or resumed during a patient's course of therapy. If the combination is formulated as separate compositions, in some embodiments, the separate compositions are provided together in a kit.

### Oncology and Oncology-Related Disorders

In accordance with the present disclosure, dispersions, compositions, or pharmaceutical compositions described herein can be used to treat or prevent cancer (e.g., carcinomas, sarcomas, leukemias, lymphomas, myelomas, etc.).

Provided herein are methods of treating cancer comprising administering a dosage form as described herein, a pharmaceutical composition as described herein, or a tablet as described herein, to a subject in need thereof.

In certain embodiments, the cancer may be locally advanced and/or unresectable, metastatic, or at risk of becoming metastatic. Alternatively, or in addition, the cancer may be recurrent or no longer responding to a treatment, such as a standard of care treatment known to one of skill in the art. In various embodiments, dispersions, compositions, or pharmaceutical compositions described herein may be used in an adjuvant setting or neoadjuvant setting. Alternatively or in addition, dispersions, compositions, or pharmaceutical compositions described herein may be used as a first line treatment, optionally in the treatment of locally advanced, unresectable, or metastatic cancer. In some embodiments, dispersions, compositions, or pharmaceutical compositions described herein can be used to treat or prevent cancer and may be used as a second line, third line, or greater line of treatment, optionally in the treatment of locally advanced, unresectable, or metastatic cancer. When indicated as a second line or greater treatment, in some embodiments an earlier line of therapy included a checkpoint inhibitor.

In some embodiments, dispersions, compositions, or pharmaceutical compositions described herein can be used to treat or prevent hematological malignancies. Exemplary types of cancer affecting the hematopoietic system include leukemias, lymphomas and myelomas, including acute myeloid leukemia, adult T-cell leukemia, T-cell large granular lymphocyte leukemia, acute lymphoblastic leukemia, chronic lymphocytic leukemia, chronic myelogenous leukemia, acute monocytic leukemia, Hodgkin's and Non-Hodgkin's lymphoma, Diffuse large B Cell lymphoma, and multiple myeloma.

In some embodiments, dispersions, compositions, or pharmaceutical compositions described herein can be used to treat or prevent solid tumors. The solid tumor may be, for example, ovarian cancer, endometrial cancer, breast cancer, lung cancer (small cell or non-small cell), colon cancer, prostate cancer, cervical cancer, biliary cancer, pancreatic cancer, gastric cancer, esophageal cancer, liver cancer (hepatocellular carcinoma), kidney cancer (renal cell carcinoma), head-and-neck tumors, mesothelioma, melanoma, sarcomas, central nervous system (CNS) hemangioblastomas, and brain tumors (e.g., gliomas, such as astrocytoma, oligodendroglioma and glioblastomas). In some embodiments, dispersions, compositions, or pharmaceutical compositions described herein can be used to treat or prevent lung cancer, genitourinary cancer, gastrointestinal cancer, or a combination thereof.

In some embodiments, the cancer is a solid tumor. In some embodiments, the cancer is breast cancer, lung cancer, gastrointestinal cancer, genitourinary cancer, or gynecological cancer. In some embodiments, the cancer is bladder cancer, breast cancer, colorectal cancer, gastric cancer, gastroesophageal cancer, lung cancer, ovarian cancer, pancreatic cancer, or prostate cancer. In some embodiments, the cancer is castrate resistant prostate cancer, esophageal adenocarcinoma, non-small cell lung carcinoma, pancreatic ductal adenocarcinoma, prostate adenocarcinoma, or urothelial cancer.

In some embodiments, dispersions, compositions, or pharmaceutical compositions described herein can be used to treat or prevent breast cancer. In further embodiments, the breast cancer is hormone receptor positive (e.g., ERα-positive breast cancer, PR-positive breast cancer, ERα-positive and PR-positive breast cancer), HER2 positive breast cancer, HER2 over-expressing breast cancer, or any combination thereof. In still further embodiments, the breast cancer is triple negative breast cancer. In still further embodiments, the breast cancer is locally advanced or metastatic triple negative breast cancer, optionally with disease progression on a prior treatment.

In some embodiments, the compounds according to this disclosure are useful in the treatment of genitourinary cancer. In further embodiments, the genitourinary cancer is gynecologic cancer. In still further embodiments, the gynecologic cancer is endometrial cancer, cervical cancer, ovarian cancer or fallopian tube carcinoma. In still further embodiments, the gynecologic cancer is locally advanced or metastatic ovarian cancer, optionally with disease progression on a prior treatment. In still further embodiments, the genitourinary cancer is urothelial carcinoma, optionally advanced or metastatic urothelial carcinoma. In some embodiments, the genitourinary cancer is advanced or metastatic MTAP-deficient urothelial carcinoma. In still further embodiments, the genitourinary cancer is prostate cancer. In still further embodiments, the genitourinary cancer is adenocarcinoma of the prostate, optionally that is eligible for radical prostatectomy. In still further embodiments, the genitourinary cancer is castration-resistant prostate cancer, optionally metastatic castrate-resistant prostate cancer. In some embodiments, dispersions, compositions, or pharmaceutical compositions described herein can be used to treat or prevent kidney cancer. In further embodiments, the kidney cancer is renal cell carcinoma. In still further embodiments, the renal cell carcinoma is clear cell renal carcinoma.

In some embodiments, the compounds according to this disclosure are useful in the treatment of kidney cancer. In further embodiments, the kidney cancer is renal cell carcinoma. In still further embodiments, the renal cell carcinoma is clear cell renal carcinoma.

In some embodiments, the compounds according to this disclosure are useful in the treatment of liver cancer. In further embodiments, the liver cancer is hepatocellular carcinoma.

In some embodiments, the compounds according to this disclosure are useful in the treatment of head and neck cancer. In further embodiments, the head and neck cancer is head and neck squamous cell carcinoma, optionally where the cancer has not been previously treated.

In some embodiments, the compounds according to this disclosure are useful in the treatment of skin cancer. In further embodiments, the skin cancer is melanoma.

In some embodiments, the compounds according to this disclosure are useful in the treatment of lung cancer. In further embodiments, the lung cancer is mesothelioma, small cell lung cancer (SCLC) or non-small cell lung cancer (NSCLC). In still further embodiments, the NSCLC is lung squamous cell carcinoma or lung adenocarcinoma. In still further embodiments, the NSCLC is nonsquamous NSCLC that is metastatic, locally advanced, or recurrent with progression.

In some embodiments, dispersions, compositions, or pharmaceutical compositions described herein can be used to treat or prevent pancreatic cancer. In further embodiments, the pancreatic cancer is pancreatic neuroendocrine tumor or pancreatic adenocarcinoma.

In some embodiments, dispersions, compositions, or pharmaceutical compositions described herein can be used to treat or prevent a neuroendocrine tumor. In further embodiments, the neuroendocrine tumor is pancreatic neuroendocrine tumor, pheochromocytoma, paraganglioma, or a tumor of the adrenal gland.

In some embodiments, dispersions, compositions, or pharmaceutical compositions described herein can be used to treat or prevent brain cancer. In further embodiments, the brain cancer is a glioma. In still further embodiments, the glioma is an astrocytoma, an oligodendroglioma, or a glioblastoma.

In some embodiments, dispersions, compositions, or pharmaceutical compositions described herein can be used to treat or prevent gastrointestinal (GI) cancer. In some embodiments, the GI cancer is a lower GI cancer, such as colon or rectal cancer. In some embodiments, the lower GI cancer is adenocarcinoma of the rectum, optionally non-metastatic adenocarcinoma of the rectum. In some embodiments, the lower GI cancer is colorectal adenocarcinoma that is metastatic, advanced, or recurrent with progression. In some embodiments, the GI cancer is an upper GI cancer, such as esophageal or gastric cancer. In further embodiments, the upper GI cancer is an adenocarcinoma, a squamous cell carcinoma, or any combination thereof. In still further embodiments, the upper GI cancer is esophageal adenocarcinoma (EAC), esophageal squamous cell carcinoma (ESCC), gastroesophageal junction adenocarcinoma (GEJ), gastric adenocarcinoma (also referred to herein as “gastric cancer”) or any combination thereof, optionally wherein the upper GI cancer is metastatic, advanced, or recurrent with progression.

The present disclosure also provides methods of treating or preventing other cancer-related diseases, disorders or conditions. The use of the term(s) cancer-related diseases, disorders and conditions is meant to refer broadly to conditions that are associated, directly or indirectly, with cancer and non-cancerous proliferative disease, and includes, e.g., angiogenesis, precancerous conditions such as dysplasia, and non-cancerous proliferative diseases disorders or conditions, such as benign proliferative breast disease and papillomas. For clarity, the term(s) cancer-related disease, disorder and condition do not include cancer per se.

In general, the disclosed methods for treating or preventing cancer, or a cancer-related disease, disorder or condition, in a subject in need thereof comprise administering to the subject a dispersion, composition, or pharmaceutical composition described herein. In some embodiments, the present disclosure provides methods for treating or preventing cancer, or a cancer-related disease, disorder or condition with a dispersion, composition, or pharmaceutical composition described herein and at least one additional therapy, examples of which are set forth elsewhere herein.

### Selection of Patients

In some instances, the methods according to this disclosure may be provided to selected patients, for example subjects identified as having in a relevant tissue or sample, e.g., detectable PD-L1 expression, microsatellite instability (MSI), deficient mismatch repair, (dMMR), high tumor mutational burden, or any combination thereof. In some instances, the subject is identified as having an oncogene driven cancer that has a mutation in at least one gene associated with the cancer.

In some embodiments, patients are selected by assessing the expression of relevant biomarkers, e.g., PD-L1 expression, microsatellite instability markers, etc., in a relevant sample, such as a peripheral blood sample or a tumor biopsy, using immunohistochemistry, immunophenotyping, PCR-based amplification, RNA sequencing, or other clinically validated assay. In one embodiment, the disclosure provides a method of treating cancer in a patient having (i) detectable PD-L1 expression, (ii) elevated PD-L1 expression, (iii) MSI-low, (iv) MSI-high, or (v) any combination of (i) to (iv) by administering a compound, dispersion, composition, or pharmaceutical composition as described herein. In another embodiment, the disclosure provides a method of treating cancer in a patient having (i) detectable PD-L1 expression, (ii) elevated PD-L1 expression, (iii) MSI-low, (iv) MSI-high, or (v) any combination of (i) to (iv) by administering a therapeutically effective amount of a compound, dispersion, composition, or pharmaceutical composition as described herein. In still another embodiment, the disclosure provides a method of administering a therapeutically effective amount of a compound, dispersion, composition, or pharmaceutical composition as described herein to an individual for the treatment of cancer based on a determination of the relative amount of PD-L1 expression. In yet another embodiment, the disclosure provides a method of administering a therapeutically effective amount of a compound, dispersion, composition, or pharmaceutical composition described herein to an individual for the treatment of cancer, the method comprising measuring PD-L1 expression and/or microsatellite instability (e.g., MSI-low or MSI-high) in a sample obtained from an individual, for example by immunohistochemistry, immunophenotyping, PCR-based amplification, or other clinically validated test, and administering a therapeutically effective amount of the compound, dispersion, composition, or pharmaceutical composition to the individual whose sample contained detectable PD-L1 expression and/or microsatellite instability. In various embodiments of the disclosure, detectable PD-L1 expression may be a tumor proportion (TPS) score of >50%, as measured by a clinically validated PD-L1 IHC assay or FDA-approved test. In various embodiments of the disclosure, detectable PD-L1 expression may be TPS score of <50%, as measured by a clinically validated PD-L1 IHC assay or FDA-approved test.

### Combination Therapy

The present disclosure contemplates the use of dispersions described herein, compositions described herein, granules described herein, pharmaceutical compositions described herein, dosage forms described herein, or tablets described herein, in combination with one or more additional therapies useful in the treatment of cancer. Some embodiments provide for a method of treating cancer in a subject, said method comprising administering to said subject a dosage form as described herein, a pharmaceutical composition as described herein, or a tablet as described herein; and at least one additional therapeutic agent; to a subject in need thereof. Exemplary therapies are described further below, as well as in WO 2018/136700 (PCT Application No. PCT/US2018/014352) and WO 2020/018680 (PCT Application No. PCT/US2019/042226), the disclosures of which are incorporated herein by reference.

In some embodiments, one or more of the additional therapies is an additional treatment modality. Exemplary treatment modalities include but are not limited to surgical resection of a tumor, bone marrow transplant, radiation therapy, and photodynamic therapy.

In some embodiments, one or more of the additional therapies is a therapeutic agent. Exemplary therapeutic agents include chemotherapeutic agents, radiopharmaceuticals, hormone therapies, epigenetic modulators, ATP-adenosine axis-targeting agents, targeted therapies, signal transduction inhibitors, RAS signaling inhibitors, PI3K inhibitors, arginase inhibitors, HIF inhibitors, AXL inhibitors, PAK4 inhibitors, immunotherapeutic agents, cellular therapies, gene therapies, immune checkpoint inhibitors, and agonists of stimulatory or co-stimulatory immune checkpoints.

In some embodiments, one or more of the additional therapeutic agents is a chemotherapeutic agent. Examples of chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas such as carmustine, chlorozotocin, fotemustine, lomustine, nimustine, and ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, pomalidomide, peplomycin, potfiromycin, puromycin, quelamycin, rodorubicin, streptonigrin, streptozocin, tubercidin, ubenimex, zinostatin, and zorubicin; anti-metabolites such as methotrexate and 5-fluorouracil (5-FU); folic acid analogs such as denopterin, methotrexate, pemetrexed, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, floxuridine, and 5-FU; androgens such as calusterone, dromostanolone propionate, epitiostanol, mepitiostane, and testolactone; anti-adrenals such as aminoglutethimide, mitotane, and trilostane; folic acid replenisher such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g., paclitaxel, nab paclitaxel, and docetaxel; chlorambucil; gemcitabine; 6-thioguanine; mercaptopurine; methotrexate; platinum and platinum coordination complexes such as cisplatin, carboplatin and oxaliplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT 11; proteasome inhibitors such as bortezomib, carfilzomib and ixazomib; topoisomerase inhibitors such as irinotecan, topotecan, etoposide, mitoxantrone, and teniposide; difluoromethylornithine (DMFO); retinoic acid; esperamicins; capecitabine; anthracyclines and pharmaceutically acceptable salts, acids or derivatives of any of the above. In certain embodiments, combination therapy comprises a chemotherapy regimen that includes one or more chemotherapeutic agents. In one embodiment, combination therapy comprises a chemotherapeutic regimen comprising one or more of FOLFOX (folinic acid, fluorouracil, and oxaliplatin), FOLFIRI (e.g., folinic acid, fluorouracil, and irinotecan), platinum and platinum coordination complexes (e.g., cisplatin, carboplatin, oxaliplatin, etc.), a taxoid (e.g., docetaxel, paclitaxel, nab-paclitaxel, etc.), and/or gemcitabine.

In some embodiments, one or more of the additional therapeutic agents is a radiopharmaceutical. A radiopharmaceutical is a form of internal radiation therapy in which a source of radiation (i.e., one or more radionuclide) is put inside a subject's body. The radiation source can be in solid or liquid form. Non-limiting examples of radiopharmaceuticals include sodium iodide I-131, radium-223 dichloride, lobenguane iodine-131, radioiodinated vesicles (e.g., saposin C-dioleoylphosphatidylserine (SapC-DOPS) nanovesicles), various forms of brachytherapy, and various forms of targeted radionuclides. Targeted radionuclides comprise a radionuclide associated (e.g., by covalent or ionic interactions) with a molecule (“a targeting agent”) that specifically binds to a target on a cell, typically a cancer cell or an immune cell. The targeting agent may be a small molecule, a saccharide (inclusive of oligosaccharides and polysaccharides), an antibody, a lipid, a protein, a peptide, a non-natural polymer, or an aptamer. In some embodiments, the targeting agent is a saccharide (inclusive of oligosaccharides and polysaccharides), a lipid, a protein, or a peptide and the target is a tumor-associated antigen (enriched but not specific to a cancer cell), a tumor-specific antigen (minimal to no expression in normal tissue), or a neo-antigen (an antigen specific to the genome of a cancer cell generated by non-synonymous mutations in the tumor cell genome). In some embodiments, the targeting agent is an antibody and the target is a tumor-associated antigen (i.e., an antigen enriched but not specific to a cancer cell), a tumor-specific antigen (i.e., an antigen with minimal to no expression in normal tissue), or a neo-antigen (i.e., an antigen specific to the genome of a cancer cell generated by non-synonymous mutations in the tumor cell genome). Non-limiting examples of targeted radionuclides include radionuclides attached to: somatostatin or peptide analogs thereof (e.g., 177Lu-Dotatate, etc.); prostate specific membrane antigen or peptide analogs thereof (e.g., 177Lu-PSMA-617, 225Ac-PSMA-617, 177Lu-PSMA-I&T, 177Lu-MIP-1095, etc.); a receptor's cognate ligand, peptide derived from the ligand, or variants thereof (e.g., 188Re-labeled VEGF₁₂₅₋₁₃₆ or variants thereof with higher affinity to VEGF receptor, etc.); antibodies targeting tumor antigens (e.g., 131I-tositumomab, 90Y-ibritumomab tiuxetan, CAM-H2-I131 (Precirix NV), I131-omburtamab, etc.).

In some embodiments, one or more of the additional therapeutic agents is a hormone therapy. Hormone therapies act to regulate or inhibit hormonal action on tumors. Examples of hormone therapies include, but are not limited to: selective estrogen receptor degraders such as fulvestrant, giredestrant, SAR439859, RG6171, AZD9833, rintodestrant, ZN-c5, LSZ102, D-0502, LY3484356, SHR9549; selective estrogen receptor modulators such as tamoxifen, raloxifene, 4-hydroxytamoxifen, trioxifene, keoxifene, and toremifene; aromatase inhibitors such as anastrozole, exemestane, letrozole and other aromatase inhibiting 4(5)-imidazoles; gonadotropin-releasing hormone agonists such as nafarelin, triptorelin, and goserelin; gonadotropin-releasing hormone antagonists such as degarelix; antiandrogens such as abiraterone, enzalutamide, apalutamide, darolutamide, flutamide, nilutamide, bicalutamide, and leuprolide; 5α-reductase inhibitors such as finasteride, and dutasteride; and the like. In certain embodiments, combination therapy comprises administration of a hormone or related hormonal agent. In one embodiment, combination therapy comprises administration of enzalutamide.

In some embodiments, one or more of the additional therapeutic agents is an epigenetic modulator. An epigenetic modulator alters an epigenetic mechanism controlling gene expression, and may be, for example, an inhibitor or activator of an epigenetic enzyme. Non-limiting examples of epigenetic modulators include DNA methyltransferase (DNMT) inhibitors, hypomethylating agents, and histone deacetylase (HDAC) inhibitors. In one or more embodiments, the dispersions, compositions, or pharmaceutical compositions described herein are combined with DNA methyltransferase (DNMT) inhibitors or hypomethylating agents. Exemplary DNMT inhibitors include decitabine, zebularine and azacitadine. In one or more embodiments, combinations of the dispersions, compositions, or pharmaceutical compositions described herein with a histone deacetylase (HDAC) inhibitor is also contemplated. Exemplary HDAC inhibitors include vorinostat, givinostat, abexinostat, panobinostat, belinostat and trichostatin A.

In some embodiments, one or more of the additional therapeutic agents is an ATP-adenosine axis-targeting agent. ATP-adenosine axis-targeting agents alter signaling mediated by adenine nucleosides and nucleotides (e.g., adenosine, AMP, ADP, ATP), for example by modulating the level of adenosine or targeting adenosine receptors. In certain embodiments, an ATP-adenosine axis-targeting agent is an inhibitor of an ectonucleotidase involved in the conversion of ATP to adenosine or an antagonist of adenosine receptor. Ectonucleotidases involved in the conversion of ATP to adenosine include the ectonucleoside triphosphate diphosphohydrolase 1 (ENTPD1, also known as CD39 or Cluster of Differentiation 39) and the ecto-5′-nucleotidase (NT5E or 5NT, also known as CD73 or Cluster of Differentiation 73). Exemplary small molecule CD73 inhibitors include CB-708, ORIC-533, LY3475070 and quemliclustat (AB680). Exemplary anti-CD39 and anti-CD73 antibodies include ES002023, TTX-030, IPH-5201, SRF-617, CPI-006, oleclumab (MED19447), NZV930, IPH5301, GS-1423, uliledlimab (TJD5, TJ004309), AB598, and BMS-986179. In one embodiment, the present disclosure contemplates combination of the dispersions, compositions, or pharmaceutical compositions described herein with a CD73 inhibitor such as those described in WO 2017/120508, WO 2018/067424, WO 2018/094148, and WO 2020/046813. In further embodiments, the CD73 inhibitor is quemliclustat.

In some embodiments, one or more of the additional therapeutic agents is a targeted therapy. In one aspect, a targeted therapy may comprise a chemotherapeutic agent, a radionuclide, a hormone therapy, or another small molecule drug attached to a targeting agent. The targeting agent may be a small molecule, a saccharide (inclusive of oligosaccharides and polysaccharides), an antibody, a lipid, a protein, a peptide, a non-natural polymer, or an aptamer. In some embodiments, the targeting agent is a saccharide (inclusive of oligosaccharides and polysaccharides), a lipid, a protein, or a peptide and the target is a tumor-associated antigen (enriched but not specific to a cancer cell), a tumor-specific antigen (minimal to no expression in normal tissue), or a neo-antigen (an antigen specific to the genome of a cancer cell generated by non-synonymous mutations in the tumor cell genome). In some embodiments, the targeting agent is an antibody and the target is a tumor-associated antigen (enriched but not specific to a cancer cell), a tumor-specific antigen (minimal to no expression in normal tissue), or a neo-antigen (an antigen specific to the genome of a cancer cell generated by non-synonymous mutations in the tumor cell genome). In some embodiments, the targeting agent is an antibody-drug conjugate comprising an antibody and a drug, wherein the antibody specifically binds to Trop-2, HER2, HER3, nectin-4, or Trop-2. Specific examples of a targeted therapy comprising an antibody and a small molecule drug include but are not limited to patritumab deruxtecan, sacituzumab govitecan-hziy, telisotuzumab vedotin, and trastuzumab deruxtecan. In other aspects, a targeted therapy may inhibit or interfere with a specific protein that helps a tumor grow and/or spread. Non-limiting examples of such targeted therapies include signal transduction inhibitors, RAS signaling inhibitors, inhibitors of oncogenic transcription factors, activators of oncogenic transcription factor repressors, angiogenesis inhibitors, immunotherapeutic agents, ATP-adenosine axis-targeting agents, AXL inhibitors, PARP inhibitors, PAK4 inhibitors, PI3K inhibitors, HIF-2a inhibitors, CD39 inhibitors, CD73 inhibitors, A2R antagonists, TIGIT antagonists, and PD-1 antagonists. ATP-adenosine axis-targeting agents are described above, while other agents are described in further detail below.

In some embodiments, one or more of the additional therapeutic agents is a signal transduction inhibitor. Signal transduction inhibitors are agents that selectively inhibit one or more steps in a signaling pathway. Signal transduction inhibitors (STIs) contemplated by the present disclosure include but are not limited to: (i) BCR-ABL kinase inhibitors (e.g., imatinib); (ii) epidermal growth factor receptor tyrosine kinase inhibitors (EGFR TKIs), including small molecule inhibitors (e.g., CLN-081, gefitinib, erlotinib, afatinib, icotinib, and osimertinib), and anti-EGFR antibodies; (iii) inhibitors of the human epidermal growth factor (HER) family of transmembrane tyrosine kinases, e.g., HER-2/neu receptor inhibitors (e.g., trastuzumab) and HER-3 receptor inhibitors; (iv) vascular endothelial growth factor receptor (VEGFR) inhibitors including small molecule inhibitors (e.g., axitinib, regorafenib, sunitinib and sorafenib), VEGF kinase inhibitors (e.g., lenvatinib, cabozantinib, pazopanib, tivozanib, XL092, etc.) and anti-VEGF antibodies (e.g., bevacizumab); (v) inhibitors of AKT family kinases or the AKT pathway (e.g., rapamycin); (vi) inhibitors of serine/threonine-protein kinase B-Raf (BRAF), such as, for example, vemurafenib, dabrafenib and encorafenib; (vii) inhibitors of rearranged during transfection (RET), including, for example, selpercatinib and pralsetinib; (viii) tyrosine-protein kinase Met (MET) inhibitors (e.g., tepotinib, tivantinib, cabozantinib and crizotinib); (ix) anaplastic lymphoma kinase (ALK) inhibitors (e.g., ensartinib, ceritinib, lorlatinib, crizotinib, and brigatinib); (x) inhibitors of the RAS signaling pathway (e.g., inhibitors of KRAS, HRAS, RAF, MEK, ERK) as described elsewhere herein; (xi) FLT-3 inhibitors (e.g., gilteritinib); (xii) inhibitors of Trop-2; (xiii) inhibitors of the JAK/STAT pathway, e.g., JAK inhibitors including tofacitinib and ruxolitinib, or STAT inhibitors such as napabucasin; (xiv) inhibitors of NF-kB; (xv) cell cycle kinase inhibitors (e.g., flavopiridol); (xvi) phosphatidyl inositol kinase (PI3K) inhibitors; (xix) protein kinase B (AKT) inhibitors (e.g., capivasertib, miransertib); (xx) platelet-derived growth factor receptor (PDGFR) inhibitors (e.g., imatinib, sunitinib, regorafenib, avapritinib, lenvatinib, nintedanib, famitinib, ponatinib, axitinib, repretinib, etc.); and (xxi) insulin-like growth factor receptor (IGFR) inhibitors (e.g., erlotinib, afatinib, gefitinib, psimertinib, dacomitinib). In some embodiments, the additional therapeutic agent comprises an inhibitor of EGFR, VEGFR, HER-2, HER-3, BRAF, RET, MET, ALK, RAS (e.g., KRAS, MEK, ERK), FLT-3, JAK, STAT, NF-kB, PI3K, AKT, or any combination thereof. In some embodiments, the additional therapeutic agent comprises an inhibitor of EGFR and/or VEGFR.

In some embodiments, one or more of the additional therapeutic agents is a RAS signaling inhibitor. Oncogenic mutations in the RAS family of genes, e.g., HRAS, KRAS, and NRAS, are associated with a variety of cancers. For example, mutations of G12C, G12D, G12V, G12A, G13D, Q61H, G13C and G12S, among others, in the KRAS family of genes have been observed in multiple tumor types. Direct and indirect inhibition strategies have been investigated for the inhibition of mutant RAS signaling. Indirect inhibitors target effectors other than RAS in the RAS signaling pathway, and include, but are not limited to, inhibitors of RAF, MEK, ERK, PI3K, PTEN, SOS (e.g., SOS1), mTORC1, SHP2 (PTPN11), and AKT. Non-limiting examples of indirect inhibitors under development include RMC-4630, RMC-5845, RMC-6291, RMC-6236, JAB-3068, JAB-3312, TNO155, RLY-1971, and B11701963. Direct inhibitors of RAS mutants have also been explored, and generally target the KRAS-GTP complex or the KRAS-GDP complex. Exemplary direct RAS inhibitors under development include, but are not limited to, sotorasib, adagrasib, mRNA-5671 and ARS1620. In some embodiments, the one or more RAS signaling inhibitors are selected from the group consisting of RAF inhibitors, MEK inhibitors, ERK inhibitors, PI3K inhibitors, PTEN inhibitors, SOS1 inhibitors, mTORC1 inhibitors, SHP2 inhibitors, and AKT inhibitors. In other embodiments, the one or more RAS signaling inhibitors directly inhibit RAS mutants.

In some embodiments, one or more of the additional therapeutic agents is an inhibitor of a phosphatidylinositol 3-kinase (PI3K), particularly an inhibitor of the PI3Kγ isoform. PI3Kγ inhibitors can stimulate an anti-cancer immune response through the modulation of myeloid cells, such as by inhibiting suppressive myeloid cells, dampening immune-suppressive tumor-infiltrating macrophages or by stimulating macrophages and dendritic cells to make cytokines that contribute to effective T cell responses thereby decreasing cancer development and spread. Exemplary PI3Kγ inhibitors include copanlisib, duvelisib. AT-104, ZX-101, tenalisib, eganelisib, SF-1126, AZD3458, and pictilisib. In some embodiments, the dispersions, compositions, or pharmaceutical compositions described herein are combined with one or more PI3Kγ inhibitor(s) described in WO 2020/0247496A1.

In some embodiments, one or more of the additional therapeutic agents is an inhibitor of arginase. Arginase has been shown to be either responsible for or participate in inflammation-triggered immune dysfunction, tumor immune escape, immunosuppression and immunopathology of infectious disease. Exemplary arginase compounds include CB-1158 and OAT-1746. In some embodiments, the dispersions, compositions, or pharmaceutical compositions described herein are combined with one or more arginase inhibitor(s) described in WO 2019/173188 and WO 2020/102646.

In some embodiments, one or more of the additional therapeutic agents is an inhibitor of an oncogenic transcription factor or an activator of an oncogenic transcription factor repressor. Suitable agents may act at the expression level (e.g., RNAi, siRNA, etc.), through physical degradation, at the protein/protein level, at the protein/DNA level, or by binding in an activation/inhibition pocket. Non-limiting examples include inhibitors of one or more subunit of the MLL complex (e.g., HDAC, DOT1L, BRD4, Menin, LEDGF, WDR5, KDM4C (JMJD2C) and PRMT1), inhibitors of hypoxia-inducible factor (HIF) transcription factor, and the like.

In some embodiments, one or more of the additional therapeutic agents is an inhibitor of a hypoxia-inducible factor (HIF) transcription factor, particularly HIF-2α. Exemplary HIF-2α inhibitors include belzutifan, ARO-HIF2, PT-2385, and those described in WO 2021113436 and WO 2021188769. In some embodiments, the dispersions, compositions, or pharmaceutical compositions described herein are combined with one or more HIF-2a inhibitors described in WO 2021188769.

In some embodiments, one or more of the additional therapeutic agents is an inhibitor of anexelekto (AXL). The AXL signaling pathway is associated with tumor growth and metastasis, and is believed to mediate resistance to a variety of cancer therapies. There are a variety of AXL inhibitors under development that also inhibit other kinases in the TAM family (i.e., TYRO3, MERTK), as well as other receptor tyrosine kinases including MET, FLT3, RON and AURORA, among others. Exemplary multikinase inhibitors include sitravatinib, rebastinib, glesatinib, gilteritinib, merestinib, cabozantinib, foretinib, BMS777607, LY2801653, S49076, and RXDX-106. AXL specific inhibitors have also been developed, e.g., small molecule inhibitors including DS-1205, SGI-7079, SLC-391, dubermatinib, bemcentinib and DP3975; anti-AXL antibodies such as ADCT-601; and antibody drug conjugates (ADCs) such as BA3011. Another strategy to inhibit AXL signaling involves targeting AXL's ligand, GAS6. For example, batiraxcept is under development as a Fc fusion protein that binds the GAS6 ligand thereby inhibiting AXL signaling. In some embodiments, the dispersions, compositions, or pharmaceutical compositions described herein are combined with one or more AXL inhibitors described in WO 2022/246177 (PCT/US2022/030227) or WO 2022/246179 (PCT/US2022/030230).

In some embodiments, one or more of the additional therapeutic agents is an inhibitor of p21-activated kinase 4 (PAK4). PAK4 overexpression has been shown across a variety of cancer types, notably including those resistant to PD-1 therapies. While no PAK4 inhibitors have been approved, some are in development, and exhibit dual PAK4/NAMPT inhibitor activity, e.g., ATG-019 and KPT-9274. In some embodiments, the dispersions, compositions, or pharmaceutical compositions described herein are combined with a PAK4 selective inhibitor. In some embodiments, the dispersions, compositions, or pharmaceutical compositions described herein are combined with a PAK4/NAMPT dual inhibitor, e.g., ATG-019 or KPT-9274.

In some embodiments, one or more of the additional therapeutic agents is (i) an agent that inhibits the enzyme poly (ADP-ribose) polymerase (e.g., olaparib, niraparib and rucaparib, etc.); (ii) an inhibitor of the Bcl-2 family of proteins (e.g., venetoclax, navitoclax, etc.); (iii) an inhibitor of MCL-1; (iv) an inhibitor of the CD47-SIRPα pathway (e.g., the anti-CD47 antibody, magrolimab, etc.); or (v) an isocitrate dehydrogenase (IDH) inhibitor, e.g., IDH-1 or IDH-2 inhibitor (e.g., ivosidenib, enasidenib, etc.).

In some embodiments, one or more of the additional therapeutic agents is an immunotherapeutic agent. Immunotherapeutic agents treat a disease by stimulating or suppressing the immune system. Immunotherapeutic agents useful in the treatment of cancers typically elicit or amplify an immune response to cancer cells. Non-limiting examples of suitable immunotherapeutic agents include: immunomodulators; cellular immunotherapies; vaccines; gene therapies; ATP-adenosine axis-targeting agents; immune checkpoint modulators; and certain signal transduction inhibitors. ATP-adenosine axis-targeting agents and signal transduction inhibitors are described above. Immunomodulators, cellular immunotherapies, vaccines, gene therapies, and immune checkpoint modulators are described further below.

In some embodiments, one or more of the additional therapeutic agents is an immunotherapeutic agent, more specifically a cytokine or chemokine, such as, IL-1, IL-2, IL-12, ELC/CCL19, SLC/CCL21, MCP-1, IL-4, IL-18, TNF, IL-15, MDC, IFNa/b, M-CSF, IL-3, GM-CSF, IL-13, and anti-IL-10; bacterial lipopolysaccharides (LPS); an organic or inorganic adjuvant that activates antigen-presenting cells and promote the presentation of antigen epitopes on major histocompatibility complex molecules agonists including, but not limited to Toll-like receptor (TLR) agonists, antagonists of the mevalonate pathway, agonists of STING; indoleamine 2,3-dioxygenase 1 (IDO1) inhibitors and immune-stimulatory oligonucleotides, as well as other T-cell adjuvants.

In some embodiments, one or more of the additional therapeutic agents is an immunotherapeutic agent, more specifically a cellular therapy. Cellular therapies are a form of treatment in which viable cells are administered to a subject. In certain embodiments, one or more of the additional therapeutic agents is a cellular immunotherapy that activates or suppresses the immune system. Cellular immunotherapies useful in the treatment of cancers typically elicit or amplify an immune response. The cells can be autologous or allogenic immune cells (e.g., monocytes, macrophages, dendritic cells, NK cells, T-cells, etc.) collected from one or more subject. Alternatively, the cells can be “(re)programmed” allogenic immune cells produced from immune precursor cells (e.g., lymphoid progenitor cells, myeloid progenitor cells, common dendritic cell precursor cells, stem cells, induced pluripotent stem cells, etc.). In some embodiments, such cells may be an expanded subset of cells with distinct effector functions and/or maturation markers (e.g., adaptive memory NK cells, tumor infiltrating lymphocytes, immature dendritic cells, monocyte-derived dendritic cells, plasmacytoid dendritic cells, conventional dendritic cells (sometimes referred to as classical dendritic cells), M1 macrophages, M2 macrophages, etc.), may be genetically modified to target the cells to a specific antigen and/or enhance the cells' anti-tumor effects (e.g., engineered T cell receptor (TCR) cellular therapies, chimeric antigen receptor (CAR) cellular therapies, lymph node homing of antigen-loaded dendritic cells, etc.), may be engineered to express of have increased expression of a tumor-associated antigen, or may be any combination thereof. Non-limiting types of cellular therapies include CAR-T cell therapy, CAR-NK cell therapy, TCR therapy, and dendritic cell vaccines. Exemplary cellular immunotherapies include sipuleucel-T, tisagenlecleucel, lisocabtagene maraleucel, idecabtagene vicleucel, brexucabtagene autoleucel, and axicabtagene ciloleucel, as well as CTX110, JCAR015, JCAR017, MB-CART19.1, MB-CART20.1, MB-CART2019.1, UniCAR02-T-CD123, BMCA-CAR-T, JNJ-68284528, BNT211, and NK-92/5.28.z.

In some embodiments, one or more of the additional therapeutic agents is an immunotherapeutic agent, more specifically a gene therapy. Gene therapies comprise recombinant nucleic acids administered to a subject or to a subject's cells ex vivo in order to modify the expression of an endogenous gene or to result in heterologous expression of a protein (e.g., small interfering RNA (siRNA) agents, double-stranded RNA (dsRNA) agents, micro RNA (miRNA) agents, viral or bacterial gene delivery, etc.), as well as gene editing therapies that may or may not comprise a nucleic acid component (e.g., meganucleases, zinc finger nucleases, TAL nucleases, CRISPR/Cas nucleases, etc.), oncolytic viruses, and the like. Non-limiting examples of gene therapies that may be useful in cancer treatment include Gendicine® (rAd-p53), Oncorine® (rAD5-H101), talimogene laherparepvec, Mx-dnG1, ARO-HIF2 (Arrowhead), quaratusugene ozeplasmid (Immunogene), CTX110 (CRISPR Therapeutics), CTX120 (CRISPR Therapeutics), and CTX130 (CRISPR Therapeutics).

In some embodiments, one or more of the additional therapeutic agent is an immunotherapeutic agent, more specifically an agent that modulates an immune checkpoint. Immune checkpoints are a set of inhibitory and stimulatory pathways that directly affect the function of immune cells (e.g., B cells, T cells, NK cells, etc.). Immune checkpoints engage when proteins on the surface of immune cells recognize and bind to their cognate ligands. The present disclosure contemplates the use of the dispersions, compositions, or pharmaceutical compositions described herein in combination with agonists of stimulatory or co-stimulatory pathways and/or antagonists of inhibitory pathways. Agonists of stimulatory or co-stimulatory pathways and antagonists of inhibitory pathways may have utility as agents to overcome distinct immune suppressive pathways within the tumor microenvironment, inhibit T regulatory cells, reverse/prevent T cell anergy or exhaustion, trigger innate immune activation and/or inflammation at tumor sites, or combinations thereof.

In some embodiments, one or more of the additional therapeutic agents is an immune checkpoint inhibitor. As used herein, the term “immune checkpoint inhibitor” refers to an antagonist of an inhibitory or co-inhibitory immune checkpoint. The terms “immune checkpoint inhibitor”, “checkpoint inhibitor” and “CPI” may be used herein interchangeably. Immune checkpoint inhibitors may antagonize an inhibitory or co-inhibitory immune checkpoint by interfering with receptor-ligand binding and/or altering receptor signaling. Examples of immune checkpoints (ligands and receptors), some of which are selectively upregulated in various types of cancer cells, that can be antagonized include PD-1 (programmed cell death protein 1); PD-L1 (PD1 ligand); BTLA (B and T lymphocyte attenuator); CTLA-4 (cytotoxic T-lymphocyte associated antigen 4); TIM-3 (T cell immunoglobulin and mucin domain containing protein 3); LAG-3 (lymphocyte activation gene 3); TIGIT (T cell immunoreceptor with Ig and ITIM domains); CD276 (B7-H3), PD-L2, Galectin 9, CEACAM-1, CD69, Galectin-1, CD113, GPR56, VISTA, 2B4, CD48, GARP, PD1H, LAIR1, TIM-1, and TIM-4, and Killer Inhibitory Receptors, which can be divided into two classes based on their structural features: i) killer cell immunoglobulin-like receptors (KIRs), and ii) C-type lectin receptors (members of the type II transmembrane receptor family). Also contemplated are other less well-defined immune checkpoints that have been described in the literature, including both receptors (e.g., the 2B4 (also known as CD244) receptor) and ligands (e.g., certain B7 family inhibitory ligands such B7-H3 (also known as CD276) and B7-H4 (also known as B7-S1, B7x and VCTN1)).

In some embodiments, an immune checkpoint inhibitor is a CTLA-4 antagonist. In further embodiments, the CTLA-4 antagonist can be an antagonistic CTLA-4 antibody. Suitable antagonistic CTLA-4 antibodies include, for example, monospecific antibodies such as ipilimumab or tremelimumab, as well as bispecific antibodies such as MEDI5752 and KN046.

In some embodiments, an immune checkpoint inhibitor is a PD-1 antagonist. In further embodiments, the PD-1 antagonist can be an antagonistic PD-1 antibody, small molecule or peptide. Suitable antagonistic PD-1 antibodies include, for example, monospecific antibodies such as balstilimab, budigalimab, camrelizumab, cosibelimab, dostarlimab, cemiplimab, ezabenlimab (BI-754091), MEDI-0680 (AMP-514; WO2012/145493), nivolumab, pembrolizumab, pidilizumab (CT-01l), pimivalimab, retifanlimab, sasanlimab, spartalizumab, sintilimab, tislelizumab, toripalimab, and zimberelimab; as well as bi-specific antibodies such as LY3434172. In still further embodiments, the PD-1 antagonist can be a recombinant protein composed of the extracellular domain of PD-L2 (B7-DC) fused to the Fc portion of IgGl (AMP-224). In certain embodiments, an immune checkpoint inhibitor is zimberelimab.

In some embodiments, an immune checkpoint inhibitor is a PD-L1 antagonist. In further embodiments, the PD-L1 antagonist can be an antagonistic PD-L1 antibody. Suitable antagonistic PD-L1 antibodies include, for example, monospecific antibodies such as avelumab, atezolizumab, durvalumab, BMS-936559, and envafolimab as well as bi-specific antibodies such as LY3434172 and KN046.

In some embodiments, an immune checkpoint inhibitor is a TIGIT antagonist. In further embodiments, the TIGIT antagonist can be an antagonistic TIGIT antibody. Suitable antagonistic anti-TIGIT antibodies include monospecific antibodies such as AGEN1327, AB308 (WO2021247591), BMS 986207, COM902, domvanalimab, EOS-448, etigilimab, IBI-929, JS006, M6223, ociperlimab, SEA-TGT, tiragolumab, vibostolimab; as well as bi-specific antibodies such as AGEN1777 and AZD2936. In certain embodiments, an immune checkpoint inhibitor is an antagonistic anti-TIGIT antibody disclosed in WO2017152088 or WO2021247591. In certain embodiments, an immune checkpoint inhibitor is domvanalimab or AB308.

In some embodiments, an immune checkpoint inhibitor is a LAG-3 antagonist. In further embodiments, the LAG-3 antagonist can be an antagonistic LAG-3 antibody. Suitable antagonistic LAG-3 antibodies include, for example, BMS-986016 (WO10/19570, WO14/08218), or IMP-731 or IMP-321 (WO08/132601, WO09/44273).

In certain embodiments, an immune checkpoint inhibitor is a B7-H3 antagonist. In further embodiments, the B7-H3 antagonist is an antagonistic B7-H3 antibody. Suitable antagonist B7-H3 antibodies include, for example, enoblituzumab, omburtumab, enoblituzumab, DS-7300a, ABBV-155, and SHR-A1811.

In some embodiments, one or more of the additional therapeutic agents activates a stimulatory or co-stimulatory immune checkpoint. Examples of stimulatory or co-stimulatory immune checkpoints (ligands and receptors) include B7-1, B7-2, CD28, 4-1BB (CD137), 4-1BBL, ICOS, ICOS-L, OX40, OX40L, GITR, GITRL, CD70, CD27, CD40, DR3 and CD2.

In some embodiments, an agent that activates a stimulatory or co-stimulatory immune checkpoint is a CD137 (4-1BB) agonist. In further embodiments, the CD137 agonist can be an agonistic CD137 antibody. Suitable CD137 antibodies include, for example, urelumab and utomilumab. In some embodiments, an agent that activates a stimulatory or co-stimulatory immune checkpoint is a GITR agonist. In further embodiments, the GITR agonist can be an agonistic GITR antibody. Suitable GITR antibodies include, for example, BMS-986153, BMS-986156, TRX-518 (WO06/105021, WO09/009116) and MK-4166 (WO11/028683). In some embodiments, an agent that activates a stimulatory or co-stimulatory immune checkpoint is an OX40 agonist. In further embodiments, the OX40 agonist can be an agonistic OX40 antibody. Suitable OX40 antibodies include, for example, MEDI-6383, MEDI-6469, MEDI-0562, PF-04518600, GSK3174998, BMS-986178, and MOXR0916. In some embodiments, an agent that activates a stimulatory or co-stimulatory immune checkpoint is a CD40 agonist. In further embodiments, the CD40 agonist can be an agonistic CD40 antibody. In some embodiments, an agent that activates a stimulatory or co-stimulatory immune checkpoint is a CD27 agonist. In further embodiments, the CD27 agonist can be an agonistic CD27 antibody. Suitable CD27 antibodies include, for example, varlilumab.

In some embodiments, one or more of the additional therapeutic agents is an agent that inhibits or depletes immune-suppressive immune cells. For example, to inhibit or deplete immunosuppressive macrophages or monocytes, the agent may be a CSF-1R antagonist such as CSF-1R antagonist antibodies including emactuzumab or cabiralizumab.

In some embodiments, each additional therapeutic agent can independently be a chemotherapeutic agent, a radiopharmaceutical, a hormone therapy, an epigenetic modulator, a targeted agent, an immunotherapeutic agent, a cellular therapy, or a gene therapy. For example, in one embodiment, the present disclosure contemplates the use of the dispersions, compositions, or pharmaceutical compositions described herein in combination with one or more chemotherapeutic agent and optionally one or more additional therapeutic agents, wherein each additional therapeutic agent is independently a radiopharmaceutical, a hormone therapy, a targeted agent, an immunotherapeutic agent, a cellular therapy, or a gene therapy. In another embodiment, the present disclosure contemplates the use of the dispersions, compositions, or pharmaceutical compositions described herein in combination with one or more chemotherapeutic agent and optionally one or more additional therapeutic agents, wherein each additional therapeutic agent is independently a targeted agent, an immunotherapeutic agent, or a cellular therapy. In another embodiment, the present disclosure contemplates the use of the dispersions, compositions, or pharmaceutical compositions described herein in combination with one or more immunotherapeutic agents and optionally one or more additional therapeutic agent, wherein each additional therapeutic agent is independently a radiopharmaceutical, a hormone therapy, a targeted agent, a chemotherapeutic agent, a cellular therapy, or a gene therapy. In another embodiment, the present disclosure contemplates the use of the dispersions, compositions, or pharmaceutical compositions described herein in combination with one or more immunotherapeutic agents and optionally one or more additional therapeutic agents, wherein each additional therapeutic agent is independently a chemotherapeutic agent, a targeted agent, or a cellular therapy. In another embodiment, the present disclosure contemplates the use of the dispersions, compositions, or pharmaceutical compositions described herein in combination with one or more immune checkpoint inhibitors and/or one or more ATP-adenosine axis-targeting agents, and optionally one or more additional therapeutic agents, wherein each additional therapeutic agent is independently a chemotherapeutic agent, a targeted agent, an immunotherapeutic agent, or a cellular therapy. In further embodiments of the above, (a) the targeted agent is a PI3K inhibitor, an arginase inhibitor, a HIF2α inhibitor, an AXL inhibitor, a PAK4 inhibitor, a VEGFR inhibitor, a VEGF kinase inhibitor, an anti-VEGF antibody, or an antibody-drug conjugate; (b) the immunotherapeutic agent is an ATP-adenosine axis-targeting agent or an immune checkpoint inhibitor; (c) the ATP-adenosine axis-targeting agent is a CD73 inhibitor or a CD39 inhibitor; (d) the ATP-adenosine axis-targeting agent is quemliclustat or AB598; (e) the immunotherapeutic agent is an anti-PD-1 antagonist antibody, an anti-PD-L1 antagonist antibody, or an anti-TIGIT antagonist antibody; (f) the immunotherapeutic agent is zimberelimab, domvanalimab, or AB308: or (g) any combination thereof. In still further embodiments of the above, the present disclosure contemplates the use of the dispersions, compositions, or pharmaceutical compositions described herein in combination with domvanalimab, etrumadenant, quemliclustat, zimberelimab, AB308, AB521, AB598, AB610, or any combination thereof.

Selection of the additional therapeutic agent(s) may be informed by current standard of care for a particular cancer and/or mutational status of a subject's cancer and/or stage of disease. Detailed standard of care guidelines are published, for example, by National Comprehensive Cancer Network (NCCN). See, for instance, NCCN Colon Cancer v 3.2021, NCCN Hepatobiliary Cancer v 5.2021, NCCN Kidney Cancer, v 3.2022, NCCN NSCLC v 7.2021, NCCN Pancreatic Adenocarcinoma v 2.2021, NCCN Esophageal and Esophagogastric Junction Cancers v 4.2021, NCCN Gastric Cancer v 5.2021, Cervical Cancer v 1.2022, Ovarian Cancer/Fallopian Tube Cancer/Primary Peritoneal Cancer v 3.2021.

### VI. Dosing

The dispersions, compositions, or pharmaceutical compositions described herein may be administered to a subject in an amount that is dependent upon, for example, the goal of administration (e.g., the degree of resolution desired); the age, weight, sex, and health and physical condition of the subject to which the formulation is being administered; the route of administration; and the nature of the disease, disorder, condition or symptom thereof. The dosing regimen may also take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan.

In general, dosing parameters dictate that the dosage amount be less than an amount that could be irreversibly toxic to the subject (the maximum tolerated dose (MTD)) and not less than an amount required to produce a measurable effect on the subject. Such amounts are determined by, for example, the pharmacokinetic and pharmacodynamic parameters associated with ADME, taking into consideration the route of administration and other factors.

An effective dose (ED), or therapeutically effective amount, is the dose or amount of an agent that produces a therapeutic response or desired effect in some fraction of the subjects taking it. The “median effective dose” or ED50 of an agent is the dose or amount of an agent that produces a therapeutic response or desired effect in 50% of the population to which it is administered. Although the ED50 is commonly used as a measure of reasonable expectance of an agent's effect, it is not necessarily the dose that a clinician might deem appropriate taking into consideration all relevant factors. Thus, in some situations, the effective amount is more than the calculated ED50, in other situations, the effective amount is less than the calculated ED50, and in still other situations, the effective amount is the same as the calculated ED50.

In addition, an effective dose of etrumadenant of the dispersions, compositions, or pharmaceutical compositions described herein may be an amount that, when administered in one or more doses to a subject, produces a desired result relative to a healthy subject. For example, for a subject experiencing a particular disorder, an effective dose may be one that improves a diagnostic parameter, measure, marker and the like of that disorder by at least about 5%, at least about 10%, at least about 20%, at least about 25%, at least about 30%, at least about 40%, at least about 50%, at least about 60%, at least about 70%, at least about 80%, at least about 90%, or more than 90%, where 100% is defined as the diagnostic parameter, measure, marker and the like exhibited by a normal subject.

In some embodiments, the dispersions, compositions, and pharmaceutical compositions of etrumadenant contemplated by the present disclosure may be administered (e.g., orally) at dosage levels of about 0.01 mg/kg to about 50 mg/kg, or about 1 mg/kg to about 25 mg/kg, of subject body weight per day, one or more times a day, to obtain the desired therapeutic effect. In some embodiments, the dispersions, compositions, and pharmaceutical compositions of etrumadenant contemplated by the present disclosure may be administered (e.g., orally) at dosage levels of about 50 mg to about 250 mg, one or more times a day, to obtain the desired therapeutic effect.

As used herein, “a total daily dosage” or “total daily dose” refers to the total amount of active agent (e.g., etrumadenant) to be administered within a 24 hour period. The total daily dosage may be administered by any method (e.g., orally) or frequency. For example, a total daily dosage of 100 mg of an active agent may be administered as 50 mg twice daily or 100 mg once daily.

In some embodiments, the dispersions, compositions, and pharmaceutical compositions of etrumadenant contemplated by the present disclosure may be orally administered to a subject in need thereof to provide a total daily dosage of about 50 mg etrumadenant to about 250 mg etrumadenant, or about 50 mg etrumadenant to about 150 mg etrumadenant. In some embodiments, the subject is administered a total daily dosage of about 50 mg, about 75 mg, about 100 mg, about 125 mg, about 150 mg, about 175 mg, about 200 mg, about 225 mg, or about 250 mg.

In some embodiments, the dispersions, compositions, and pharmaceutical compositions of etrumadenant contemplated by the present disclosure may be administered (e.g., orally) at a dosage level described above without dose adjustment or discontinuation due to concomitant use of an inhibitor or an inducer of a cytochrome P450 (CYP) enzyme, an inhibitor or inducer of P-glycoprotein (P-gp), or an inhibitor or inducer of breast cancer resistance protein (BCRP). P-gp and BCRP are efflux transporters expressed in the gastrointestinal tract and can affect oral bioavailability of drugs. CYP enzymes are catalyzing enzymes, and their inhibition or inducement can affect bioavailability of drugs by altering the rate of the drugs metabolism. The most common CYP enzymes involved in drug metabolism include enzymes isoforms of CYP1A2, CYP2B6, CYP2C8, CYP2C9, CYP2C19, 256 CYP2D6, and CYP3A. CYP inhibitors can be classified as strong, moderate or weak based on the inhibitor's effect on an index substrate. Index CYP substrates are known in the art. See, for example, the International Council for Harmonisation (ICH) M12 Guideline on Drug Interaction Studies released Jul. 21, 2022. A subject's genotype can also affect CYP activity. Per the United States Food and Drug Administration, normal metabolizers are subjects who do not have genetic variants that are expected to impact metabolism, ultrarapid metabolizers are subjects who generally have two or more copies of a genetic variant that increases metabolic function, intermediate metabolizers are subjects who generally have one or two copies of a genetic variant that reduces the ability to metabolize a drug, and poor metabolizers are subjects who generally have two copies of a genetic variant that results in little to no ability to metabolize a drug (www.fda.gov/medical-devices/precision-medicine/table-pharmacogenetic-associations).

In vitro data suggested etrumadenant metabolism was mediated by CYP3A4, CYP2C8, and uridine 5′-diphospho glucuronosyltransferase (UGT) enzyme isoforms and that etrumadenant was a substrate of P-gp and breast cancer resistance protein (BCRP). However, as further described herein, in humans, co-administration of a strong CYP3A4 inhibitor with etrumadenant had a limited effect on the pharmacokinetics of etrumadenant and co-administration of a P-gp inhibitor with etrumadenant may not influence etrumadenant's oral absorption. Accordingly, in some embodiments, the dispersions, compositions, and pharmaceutical compositions of etrumadenant contemplated by the present disclosure may be administered (e.g., orally) at a dosage level described above without dose adjustment or discontinuation due to concomitant use of a CYP3A4 inhibitor or a P-gp inhibitor or without dose adjustment or discontinuation due to the subject being a poor CYP3A4 metabolizer.

Some embodiments provide for a method of treating cancer in a subject concomitantly receiving a CYP3A4 inhibitor or a P-gp inhibitor or in a subject that is a poor CYP3A4 metabolizer, said method comprising administering to said subject a dosage form as described herein, a pharmaceutical composition as described herein, or a tablet as described herein to a subject in need thereof. Some embodiments provide for a method of treating a disease, disorder, or condition, mediated at least in part by the adenosine A_{2A} receptor (A_{2A}R) or the adenosine A_{2B} receptor (A_{2B}R) in a patient, wherein said patient is concomitantly receiving a CYP3A4 inhibitor or a P-gp inhibitor or the subject is a poor CYP3A4 metabolizer, the method comprising administering a therapeutically effective amount of etrumadenant. In some embodiments, the CYP3A4 inhibitor is a strong CYP3A4 inhibitor. In some embodiments, the CYP3A4 inhibitor is a moderate CYP3A4 inhibitor. In some embodiments, the CYP3A4 inhibitor is a weak CYP3A4 inhibitor.

For administration of an oral agent, the compositions can be provided in the form of tablets, capsules and the like containing from 1.0 to 1000.0 milligrams of the active ingredient, particularly 1.0, 3.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient. In some embodiments, the tablet or capsule described herein comprises 10 mg to 100 mg of etrumadenant or a pharmaceutically acceptable salt thereof. In some embodiments, the tablet or capsule described herein comprises 50 mg to 100 mg of etrumadenant or a pharmaceutically acceptable salt thereof. In some embodiments, the tablet or capsule described herein comprises 75 mg of etrumadenant or a pharmaceutically acceptable salt thereof. In some embodiments, the tablets or capsules described herein are administered once daily to obtain the desired therapeutic effect.

In some embodiments, a tablet as described herein comprises 50 mg of etrumadenant. In some embodiments, a tablet as described herein comprises 75 mg of etrumadenant. In some embodiments, a tablet as described herein comprises 100 mg of etrumadenant. In some embodiments, a tablet as described herein comprises 150 mg of etrumadenant.

In some embodiments, the dosage of the dispersions or compositions of etrumadenant, or a pharmaceutically acceptable salt thereof, is contained in a “unit dosage form.” The phrase “unit dosage form” refers to physically discrete units, each unit containing a predetermined amount of etrumadenant, either alone or in combination with one or more additional agents, sufficient to produce the desired effect. It will be appreciated that the parameters of a unit dosage form will depend on the particular agent and the effect to be achieved.

### Exemplary Dosing

Some embodiments provide for methods of treating a disease, disorder, or condition, mediated at least in part by the adenosine A_{2A} receptor (A_{2A}R), at least in part by the adenosine A_{2B} receptor (A_{2B}R), or at least in part by both A_{2A}R and A_{2A}R receptors, said method comprising administering to a subject in need thereof a dosage form, a pharmaceutical composition, or a tablet of this disclosure.

Some embodiments provide for methods of treating cancer comprising administering to a subject in need thereof a dosage form, a pharmaceutical composition, or a tablet of this disclosure. In some embodiments, the cancer is a solid tumor. In some embodiments, the cancer is breast cancer, lung cancer, gastrointestinal cancer, genitourinary cancer, or gynecological cancer. In some embodiments, the cancer is bladder cancer, breast cancer, colorectal cancer, gastric cancer, gastroesophageal cancer, lung cancer, ovarian cancer, pancreatic cancer, or prostate cancer. In some embodiments, the cancer is castrate resistant prostate cancer, esophageal adenocarcinoma, non-small cell lung carcinoma, pancreatic ductal adenocarcinoma, prostate adenocarcinoma, or urothelial cancer. In some embodiments, the cancer is locally advanced, unresectable, or metastatic cancer.

In some embodiments, the pharmaceutical composition is a formulation of Table 16, Table 19, Table 20, or Table 24. In some embodiments, the tablet is a tablet of Table 16. Table 19, Table 20, or Table 24.

In some embodiments, the subject is administered a total daily dosage of about 50 mg etrumadenant to about 250 mg etrumadenant, or about 50 mg etrumadenant to about 150 mg etrumadenant, optionally wherein the total daily dosage is administered once a day.

In some embodiments, the subject is administered a total daily dosage of about 50 mg, about 75 mg, about 100 mg, about 125 mg, or about 150 mg.

In some embodiments, the total daily dosage of etrumadenant is not adjusted for a subject that is a poor CYP3A4 metabolizer or for a subject concomitantly taking a CYP3A4 inhibitor (e.g., a weak, moderate, or strong CYP3A4 inhibitor) or a P-gp inhibitor.

In some embodiments, a subject is further administered one or more than one additional therapy (e.g., 1, 2, 3, etc.), optionally selected from radiation therapy, a chemotherapeutic agent, a checkpoint inhibitor, an ATP-adenosine axis-targeting agent, a PI3K inhibitor, an arginase inhibitor, a HIF2α inhibitor, an AXL inhibitor, a PAK4 inhibitor, a VEGFR inhibitor, a VEGF kinase inhibitor, an anti-VEGF antibody, and an antibody-drug conjugate.

In some embodiments, a subject is further administered one or more than one additional therapy (e.g., 1, 2, 3, etc.), optionally selected from radiation therapy, a chemotherapeutic agent, AB308, AB521, AB598, AB801, domvanalimab, quemliclustat, and sacituzumab govitecan.

In embodiments where the subject is administered one or more than one chemotherapeutic agent, wherein the one or more chemotherapeutic agent may be FOLFOX, FOLFIRI, CAPOX, a platinum or platinum coordination complex (e.g., cisplatin, carboplatin, oxaliplatin, etc.) a taxoid (e.g., docetaxel, paclitaxel, nab-paclitaxel, etc.), gemcitabine, a folic acid analog (e.g., pemetrexed, etc.), or an antiandrogen (e.g., enzalutamide, etc.).

### VII. Kits

The present disclosure also contemplates kits comprising dispersions, compositions, or pharmaceutical compositions described herein. The kits are generally in the form of a physical structure housing various components, as described below, and may be utilized, for example, in practicing the methods described above.

A kit can include one or more of the dispersions, compositions, or pharmaceutical compositions described herein (provided in, e.g., a sterile container). The dispersions, compositions, or pharmaceutical compositions described herein can be provided in a form that is ready for use (e.g., a tablet or capsule) or in a form requiring, for example, reconstitution or dilution (e.g., a powder) prior to administration. When the dispersions, compositions, or pharmaceutical compositions described herein are in a form that needs to be reconstituted or diluted by a user, the kit may also include diluents (e.g., sterile water), buffers, pharmaceutically acceptable excipients, and the like, packaged with or separately from the dispersions, compositions, or pharmaceutical compositions described herein. When combination therapy is contemplated, the kit may contain the several agents separately or they may already be combined in the kit. Each component of the kit may be enclosed within an individual container, and all of the various containers may be within a single package. A kit of the present disclosure may be designed for conditions necessary to properly maintain the components housed therein (e.g., refrigeration or freezing).

A kit may contain a label or packaging insert including identifying information for the components therein and instructions for their use (e.g., dosing parameters, clinical pharmacology of the active ingredient(s), including mechanism of action, pharmacokinetics and pharmacodynamics, adverse effects, contraindications, etc.). Labels or inserts can include manufacturer information such as lot numbers and expiration dates. The label or packaging insert may be, e.g., integrated into the physical structure housing the components, contained separately within the physical structure, or affixed to a component of the kit (e.g., an ampule, tube or vial).

Labels or inserts can additionally include, or be incorporated into, a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media or memory-type cards. In some embodiments, the actual instructions are not present in the kit, but means for obtaining the instructions from a remote source, e.g., via the internet, are provided.

### EXAMPLES

The following examples are put forth so as to provide those of ordinary skill in the art with a complete disclosure and description of how to make and use the present disclosure, and are not intended to limit the scope of what the inventors regard as their disclosure. Efforts have been made to ensure accuracy with respect to numbers used (e.g., amounts, temperature, etc.), but some experimental errors and deviations should be accounted for.

Unless indicated otherwise, temperature is in degrees Celsius (° C.), and pressure is at or near atmospheric. Standard abbreviations are used, including the following: rt or r.t.=room temperature; min=minute(s); h or hr=hour(s); ng=nanogram; μg=microgram; mg=milligram; g=gram; kg=kilogram; μl or μL=microliter; ml or mL=milliliter; 1 or L=liter, μM=micromolar; mM=millimolar; M=molar; mol=mole; mmol=millimole; nM=nanomolar; gA=gram of Active; mgA=milligram of Active; μgA=microgram of Active; HPMCAS=hydroxypropyl methylcellulose acetate succinate or hypromellose acetate succinate; CAP=cellulose acetate phthalate; PVP-VA=polyvinylpyrrolidone/vinyl acetate copolymer (copovidone); FaSSIF=fasted state simulated intestinal fluid.

### Example 1

The thermal properties and x-ray diffraction pattern of the starting drug substance used in the following examples was characterized by differential scanning calorimeter (DSC) and x-ray powder diffraction (XRPD).

Thermal Properties. Thermal properties were measured using a TA Instruments Discovery DSC2500 DSC equipped with a TA instruments Refrigerated Cooling System 90 operating in modulated mode. Briefly, bulk drug substance was analyzed by standard DSC with a heating rate of 10° C. per minute ramping up to 220° C. Amorphous etrumadenant was created from the starting drug substance by rapidly melt quenching using liquid N₂ and then analyzed by modulated DSC (MDSC). Events monitored include the glass transition temperature (T_{g}), cold crystallization (T_{c}), defined as a crystallization event at a temperature lower than the melt temperature, and melting temperature (Tₘ). In one experiment, the thermal properties were as follows: T_{g}=69° C.; T_{c}=no crystallization up to 220° C.; Tₘ=192° C. Minor lot-to-lot variation may occur.

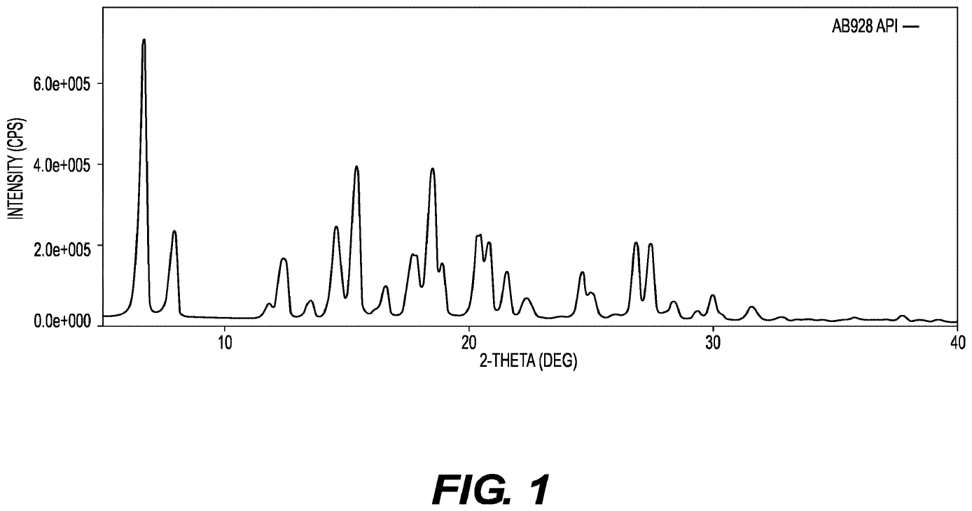

XRPD. The diffraction pattern of the starting drug substance was obtained by XRPD. XRPD was performed using a Rigaku Miniflex 6G X-ray diffractometer. Samples were irradiated with monochromatized Cu Kα radiation and analyzed between 5° and 40° with a continuous scanning mode. Samples were rotated during analysis to minimize preferred orientation effects. A summary of the XRPD analysis parameters can be found in Table 1. The diffraction pattern of the starting drug substance indicates that the starting drug substance was a crystalline material (FIG. 1), consistent with the thermal analysis.

TABLE 1
XRPD Analysis Parameters

| Parameter | Value | |
|---|---|---|
| Instrument | Rigaku Miniflex 6G | |
| Radiation Source | Cu-Kα (1.5406 Å), Line Focus 0.4 mm × | |
| | 12 mm | |
| Scan Type | Coupled 2θ/θ | |
| Scan Range | 5°-40° | |
| Step Increment | 0.005° | |
| Ramp Rate | 0.9°/min | |
| Voltage | 40 | kV |
| Current | 15 | mA |
| Rotation | 30 | rpm |
| Holder | Zero-Background Cup | |
| Divergent Slit Width | 0.625 | mm |

### Example 2: Comparative Etrumadenant Tablets Containing Crystalline API

Etrumadenant tablets were manufactured to contain 50 mg etrumadenant crystalline free base. Tablets were prepared as follows. API was blended with intragranular components in a suitable blender. The blend was de-lumped and then subject to compaction and milled into granules. Extragranular components were then blended with the granules and the mixture subject to tableting. Tablet composition is provided in Table 2.

TABLE 2
Etrumadenant Tablet Composition

| | |
|---|---|
| Material | Composition (% w/w) |

Intra-Granular

| | |
|---|---|
| AB928 | 10 |
| Microcrystalline cellulose | 42 |
| Mannitol | 42 |
| Croscarmellose sodium | 5 |
| Magnesium Stearate | 0.5 |

Extra-Granular

| | |
|---|---|
| Magnesium Stearate | 0.5 |
| Core Tablet Total | 100.0 |

### Example 3: Capsules of Etrumadenant

The chemical stability of etrumadenant in a number of semi-solid fill formulations was examined at 5% drug load. Briefly, vehicle compositions were prepared by melting selected excipients with mixing. Starting drug substance was then added, and the formulation mixed with continued heating until a clear solution was formed. The solution was then filled into capsules and subsequently sealed using a gelatin banding. The gelatin banding solution was prepared by mixing gelatin into a solution of polysorbate 80 and water until the mixture was uniform.

Vehicle compositions included PEG 400, PEG 1500, or PEG 4000 with Solutol HS 15, Kolliphor RH40, or Gelucire 44/14 in ratios ranging from 1:1 to 3:1. All semi-solid fill formulations tested achieved enhanced solubility as compared to starting drug substance in water, and were chemically stable when stored in closed containers at 2-8° C., 25° C., and 40° C./75% relative humidity (RH) for various lengths of time (e.g., 1 day, 7 days, 1 month, 2 months). Based on in vitro dissolution studies and nonclinical pharmacokinetic (PK) studies, a capsule containing 25 mg etrumadenant, and PEG 1500:Kolliphor RH40 (3:1) was selected as a clinical formulation. An exemplary formulation is provided in Table 3.

TABLE 3
Etrumadenant Capsule, 25 mg

| Material | Composition (% w/w) |
|---|---|
| Etrumadenant | 4.55 |
| Polyethylene glycol 1500 | 71.58 |
| Polyoxyl 40 hydrogenated castor oil | 23.87 |
| Total (capsule content) | 100 |
| Gelatin capsule, light blue opaque, size 0 | — |
| Gelatin band | — |
| Total | — |

### Example 4: Exemplary Solid Dispersions of Etrumadenant

The kinetic solubility of etrumadenant and supersaturation maintenance was first measured in the presence of select cellulose-based and vinyl-pyrrolidone-based polymer excipients. Solvent shift dissolution experiments were performed as follows. Starting drug substance was dissolved in dimethylsulfoxide (DMSO) at 200 mg/mL to produce a drug stock solution. Each polymer was dissolved at 1 mg/mL and 3 mg/mL in FaSSIF (2 mg/mL simulated intestinal fluids (SIF) in 100 mM phosphate buffered solution (PBS), pH 6.8), prepared per the manufacturer's directions. Polymers tested included cellulose acetate phthalate (CAP), hydroxypropyl methylcellulose E3 (HPMC E3LV), hydroxypropyl methylcellulose acetate succinate, grade H (HPMCAS-H), HPMCAS, grade L (HPMCAS-L), HPMCAS, grade M (HPMCAS-M), hydroxypropyl methylcellulose phthalate (HPMCP), polyvinylpyrrolidone (PVP), polyvinylpyrrolidone/vinyl acetate copolymer (copovidone) (PVP-VA), and Soluplus®. 25 μL of drug stock solution was then introduced into 10 mL of polymer FaSSIF solution, or into 10 mL of FaSSIF without polymer as a control, while stirring with a magnetic stir bar at 300 rpm (dilution of etrumadenant to 1 mg/mL). 0.5 mL aliquots were taken at the following time points: 5, 15, 30, 45, and 60 minutes without media replacement. Aliquots were transferred to a 1.5 mL centrifuge tube and spun down at 13,000 rpm for 3 min. 100 μL of supernatant was sampled and diluted with 900 μL 3:1 ACN:H₂O for HPLC analysis using the parameters listed in Table 4.

TABLE 4
HPLC Parameters for Assay and Related Substance Analysis

| Parameter | Value | | |
|---|---|---|---|
| Instrument | Agilent 1100 or 1200 Series HPLC system, | | |
| | or other suitable HPLC system, with a UV | | |
| | detector, capable of gradient mixing and | | |
| | associated chromatography data system. | | |
| Column | YMC Pack ODS-AQ, 4.6 × 250 mm, 5 μm, | | |
| | 20 nm | | |
| | PN: AQ20S05-2546WT | | |
| Mobile Phase A | 0.1% Trifluoracetic acid (TFA) in H₂O (v/v) | | |
| (MPA) | | | |
| Mobile Phase B | 0.1% TFA in Acetonitrile (ACN) (v/v) | | |
| (MPB) | | | |
| Program Type | Time(min) | % MPA | % MPB |
| | 0 | 95 | 5 |
| | 25.0 | 2 | 98 |
| | 36.0 | 2 | 98 |
| | 36.1 | 95 | 5 |
| | 48.0 | 95 | 5 |
| Flow Rate | 0.8 mL/min | | |
| Column Temperature | 30° C. | | |
| Sample Temperature | Room temperature | | |
| Injection Volume | 5 μL | | |
| Detection Wavelength | 280 nm (BW: 4 nm, SW: 4 nm, Ref: Off) | | |
| Run Time | 48 min | | |
| Diluent | 0.1% TFA in 3:1 ACN:H₂O (v/v) | | |
| API STD1 | 0.5 mg/mL | | |
| Concentration | | | |
| Needle Wash | Diluent | | |
| Etrumadenant | ~15.2 min | | |
| Retention Time | | | |

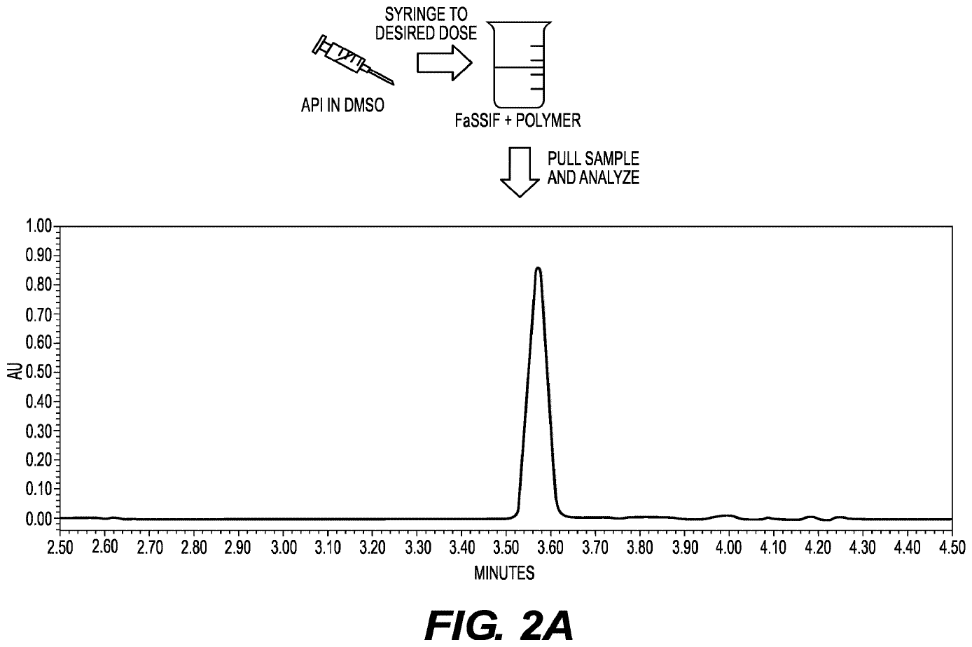

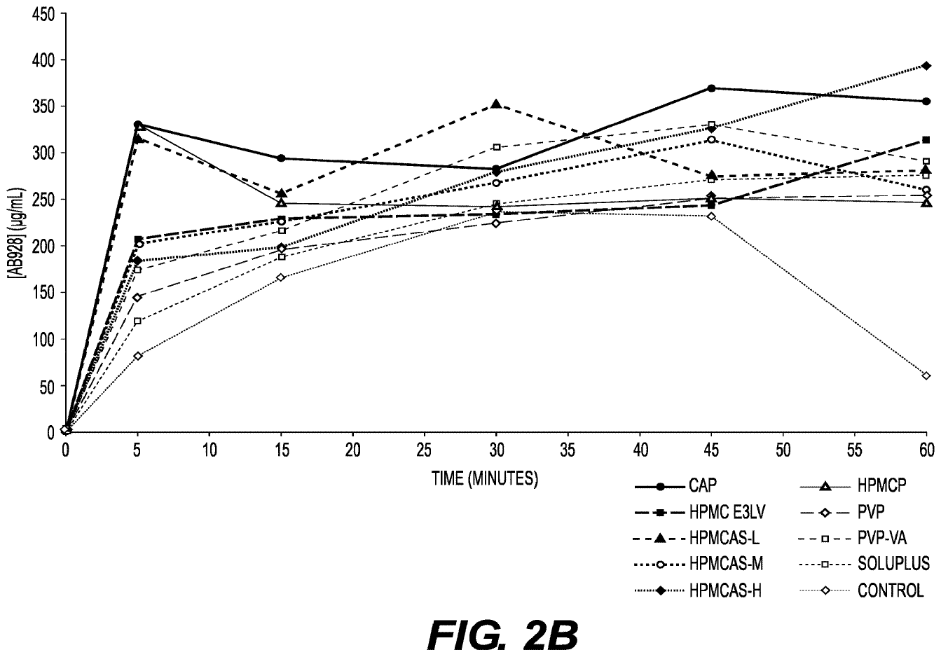

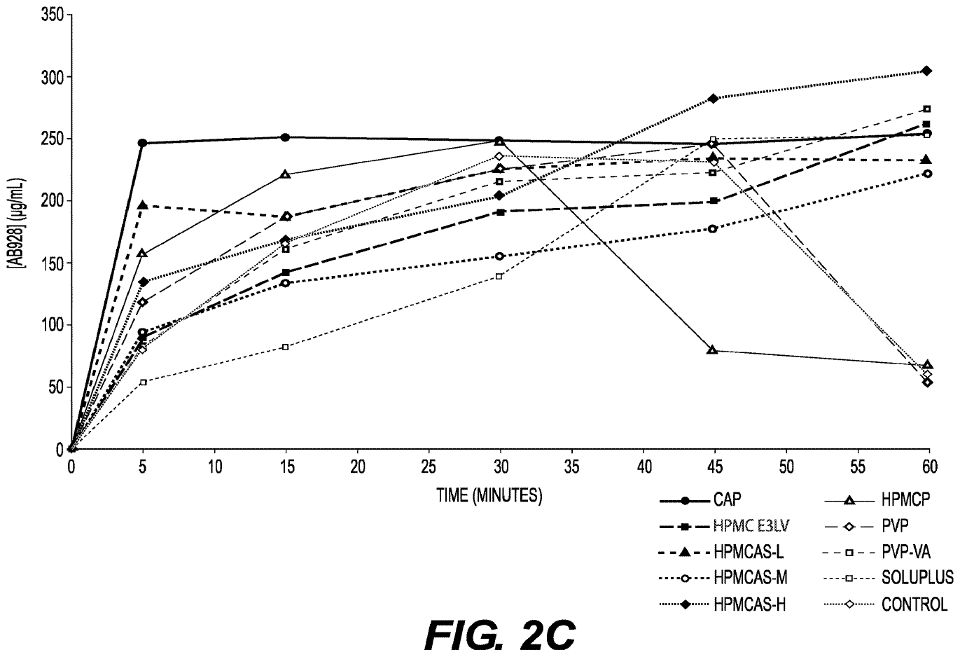

The measured etrumadenant concentrations in the presence of the polymer were compared to the measured etrumadenant concentration without any polymer present in FaSSIF. At 25% drug loading, i.e., a 1:3 drug to polymer ratio (w/w), all polymers resulted in prolonged supersaturation of etrumadenant throughout 60 minutes when compared to the control, which began to precipitate out after the 45-minute timepoint. At 50% drug loading, i.e., a 1:1 drug to polymer ratio (w/w), all polymers gave improved sustainment of supersaturation with the exception of PVP and HPMCP. 25% drug loading showed higher degrees of supersaturation than amorphous etrumadenant and when compared to 50% drug load. Data are shown in FIG. 2B and FIG. 2C.

Eight solid dispersions prepared with different ratios of starting drug substance to polymer (25:75 w/w or 40:60 w/w) and different polymers (HPMCAS-L, HPMCAS-M, CAP, or PVP-VA) were then characterized. In this example, and throughout the following examples, solid dispersions (also referred to as SDIs) are identified using a nomenclature wherein the weight ratio of drug to polymer(s) is identified, followed by identification of the drug and polymer(s). As an example, “25:75 AB928:HPMCAS-M SDI” refers to a solid dispersion prepared using 25% by weight AB928 (etrumadenant) and 75% by weight HPMCAS-M. Stated another way, 100% of the weight of the solids used to produce the dispersion can be attributed to etrumadenant and HMPCAS-M, and the etrumadenant and HMPCAS-M are in a 1:3 weight ratio. The dispersion produced does not contain exactly 25% by weight etrumadenant and 75% by weight HPMCAS-M, however, as residual solvent, water and impurities may account for some of the dispersion's weight.

Each of the eight formulations were spray dried from neat acetone. Briefly, starting drug substance and one of the four polymers in the preceding paragraph were added into the spray solvent (acetone) at 10% (w/w) solids loading and mixed at 20-25° C. until complete dissolution was achieved to produce a spray solution. The spray solution was then spray dried by atomizing the spray solution into a drying chamber where solvent was removed in the carrier gas and SDI powder was produced. Spray drying parameters are provided in Table 5. The SDI powder then underwent secondary drying to remove residual solvent.

TABLE 5
Summary of Spray Drying Parameters

| Parameter | Value | |
|---|---|---|
| Spray Dryer | Buchi B290 | |
| Cyclone | High Efficiency | |
| Solvent | Acetone | |
| Batch Size, Active (A) | 1.5 | gA |
| Solution Composition | 10% solids | |
| Atomization Pressure | 28 | psi |
| Solution Feed Rate | ~18-20 | g/min |
| Inlet Temperature | 84-88° | C. |
| Outlet Temperature | 41-43° | C. |

| | |
|---|---|
| Secondary Drying | 23 hr 30 min at 40° C. |

The resulting SDI powder was characterized by a variety of analytical methods including powder X-ray diffraction (XRPD), scanning electron microscopy (SEM), modulated differential scanning calorimetry (MDSC), residual solvents by gas chromatography headspace sampling (GC-HS), assay and related substances by high-performance liquid chromatography (HPLC), water content by Karl Fisher titration (KF), and non-sink dissolution.

GC-HS was used to measure the residual acetone remaining after secondary drying. Measurements were made using an HP 6890 series GC equipped with an Agilent 7697A headspace sampler. A 30 m×0.32 mm×1.8μ capillary column with 6% cyanopropylphenyl 94% dimethylpolysiloxane GC column was used for the testing. GC samples were prepared by dissolving ˜100 mg sample in 4 mL dimethyl sulfoxide (DMSO). The residual solvent in all formulations was well below the acetone limit (5000 ppm) set forth by the International Conference on Harmonization (ICH).

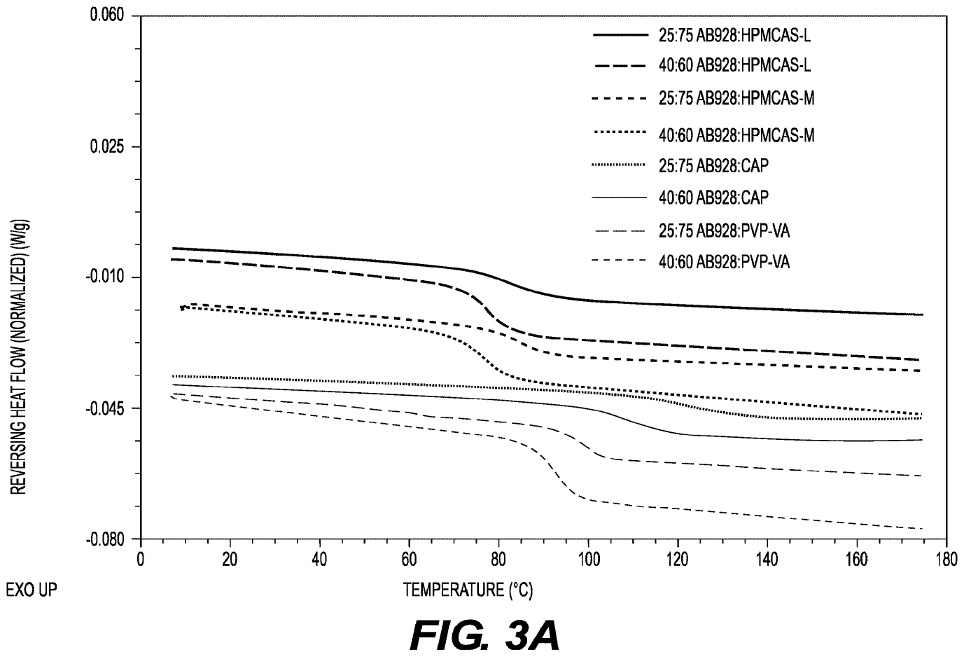

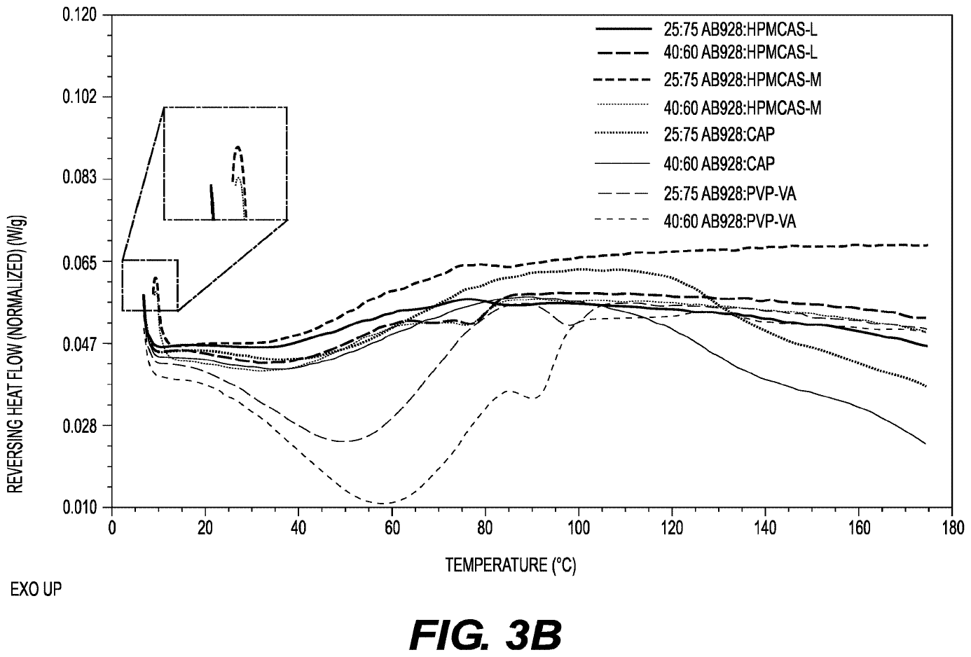

Thermal analysis by MDSC, performed as described in Example 1, showed that all dispersions (see FIG. 3 legend for dispersions) had a single, relatively high glass transition temperature (T_{g}) (FIG. 3), ranging from about 76° C. to about 123° C. The presence of a single T_{g} indicated an intimately mixed amorphous solid dispersion with good homogeneity, while the high T_{g} indicated good physical stability of the dispersion, e.g., the propensity of etrumadenant to recrystallize during long-term storage was low.

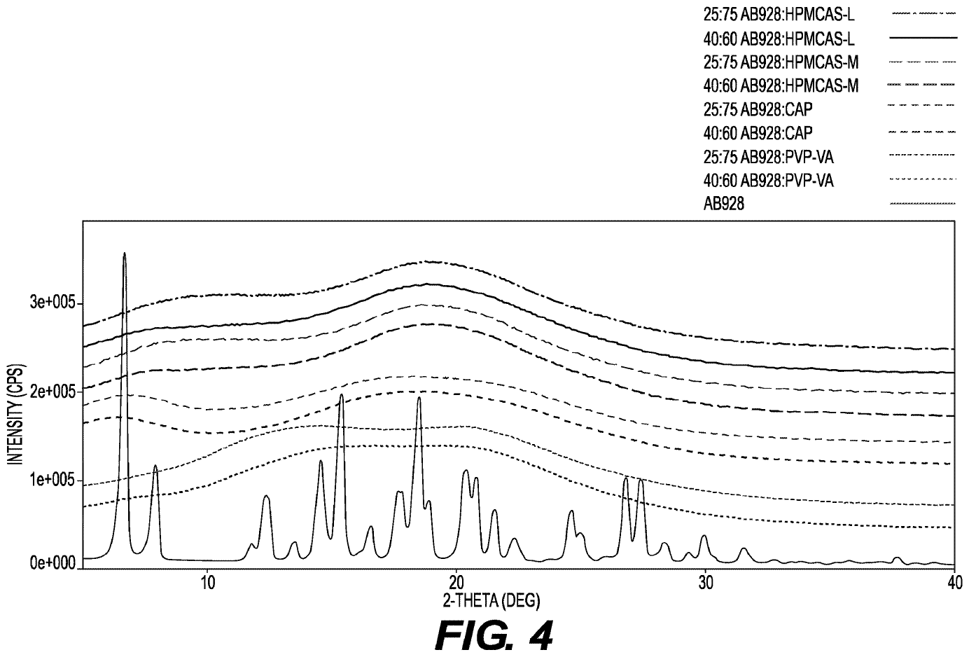

To further evaluate the crystallinity of the spray-dried particles, samples of the SDI powders were analyzed by XRPD as described in Example 1. Amorphous materials give an “amorphous halo” diffraction pattern, absent of discrete peaks that would be found in a crystalline material. Characterization by XRPD indicated that the eight dispersions were amorphous, and no crystalline peaks were observed in the diffractograms (FIG. 4).

Surface morphology of the spray-dried particles was characterized using SEM. SEM samples were prepared by dispersing SDI powder onto an adhesive carbon-coated sample stub and coating with a thin conductive layer of gold using a Cressington 108 Auto. Samples were analyzed using a FEI Quanta 200 SEM fitted with an Everhart-Thomley (secondary electron) detector operating in high vacuum mode. Micrographs at various magnifications were captured for qualitative particle morphology analysis. Experimental parameters including spot size, working distance, and acceleration voltage were varied from sample to sample to obtain the best imaging conditions. For each dispersion, the morphology observed consisted of whole and collapsed spheres with smooth surfaces. No crystalline material was observed in any samples.

SDI powders collected after secondary drying were analyzed for water content by a Metrohm 831 Karl Fischer Coulometric Titrator with a Metrohm 874 oven processor. About 100 mg samples were sealed in 6 mL crimp vials followed by measurement of water content with the following parameters: Reagent Hydranal Coulomat AG-Oven, oven temperature 130° C. and sample extraction time 300 seconds. Water content was typically less than 1% by weight.

In vitro drug dissolution performance for each dispersion was evaluated under non-sink conditions in a two-stage process that simulates pH and bile salt concentrations for both gastric and intestinal exposure. In vitro dissolution testing under non-sink conditions allows for a direct evaluation of each dispersion's ability to generate and maintain supersaturation above crystalline etrumadenant's solubility; was used as a predictive surrogate for ensuring product quality and in vivo performance; and is an effective test to discriminate between formulations at a given stability condition.

To begin dissolution (t=0), a sample of SDI powder was briefly suspended in 4 mL of FaSSIF (2 mg/mL SIF in 100 mM PBS) and then transferred into 50 mL SGF (0.1 N HCl (aq)) pre-heated to 37° C. in a USP Type 2 mini-vessel (100 mL total vessel volume) with stirring (paddles) at 100 rpm. At 30 minutes, a gastric transfer step was performed wherein the volume in each dissolution vessel was approximately doubled using 2×FaSSIF (4.48 mg/mL SIF powder (BioRelevant)), resulting in a final pH of 6.8 in a total volume of 100 mL. Samples taken before and after this transfer at indicated times were evaluated for drug concentration by HPLC. The drug concentrations measured in this test are a composite of SDI free drug, drug in micelles, and drug suspended in solution. A summary of the dissolution and HPLC parameters are shown in Table 6 and Table 7, respectively.

TABLE 6
Summary of Non-Sink Dissolution Parameters

| Parameter | Value | |
|---|---|---|
| Dissolution | Mini-paddles and 100 mL volume vessels | |
| Medium | | |
| Dose | 0.1N HCl → 2.24 mg/mL FaSSIF gastric transfer | |
| Gastric Transfer | 1.0 mgA/mL (SGF) → 0.5 mgA/mL (FaSSIF) | |
| Medium | 30 | min |
| Temperature | | |
| Medium Volume | 37° C. (±0.5° C.) | |
| Vessel Replicates | N = 3 | |
| Dilution Factor | 10x, 100 μL supernatant → 900 μL diluent | |
| Stir Rate | 100 | RPM |
| Paddle Height | 20 | minutes |
| Sampling Volume | 1.0 | mL |
| Sample Isolation | Centrifuge samples at ~13,000 RCF for 3 minutes and | |
| | isolate supernatant for HPLC analysis | |
| Sampling Time | 10, 25, 35, 50, 70, 120, and 210 minutes | |
| Points | | |

TABLE 7
HPLC Parameters for Non-Sink Dissolution Test

| Parameter | Value | |
|---|---|---|
| Instrument | Agilent 1100 or 1200 Series HPLC system, or | |
| | other suitable HPLC system, with a UV | |
| | detector, capable of gradient mixing and | |
| | associated chromatography data system. | |
| Column | Agilent Poroshell 120 EC-C18, 2.7 μm, 4.6 × | |
| | 50 mm | |
| | PN: 699975-902(T) | |
| Mobile Phase A | 0.05% TFA in H₂O (v/v) | |
| Mobile Phase B | 0.05% TFA in ACN (v/v) | |
| Program Type | 65:35 (A:B) Isocratic | |
| Flow Rate | 1.0 | L/min |
| Column Temperature | 30° | C. |
| Sample Temperature | Room temperature | |
| Injection Volume | 5 | μL |
| Detection Wavelength | 258 nm (BW: 4 nm, SW: 4 nm, Ref: Off) | |
| Run Time | 3 | min |
| Diluent | 3:1 ACN:H₂O (v/v) | |
| Etrumadenant STD1 | 0.5 | mg/mL |
| Concentration | | |
| Needle Wash | Diluent | |
| Etrumadenant | 1.6 | min |
| Retention Time | | |

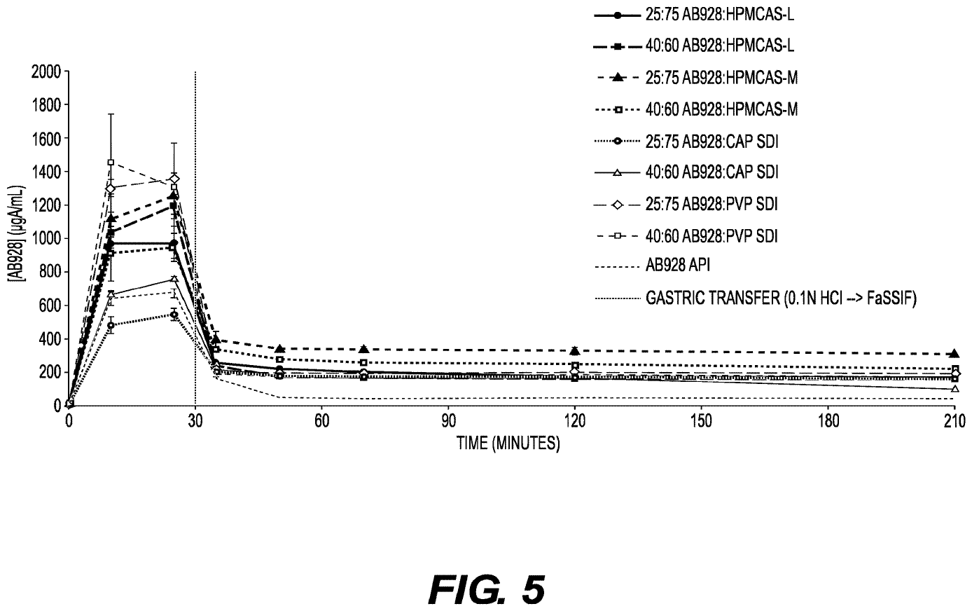

All of the dispersions exhibit improved in vitro non-sink dissolution performance relative to crystalline etrumadenant (FIG. 5 and Table 8). Predicted gastric solubility in this model was varied among the formulations, with a low correlation between gastric dissolution levels and subsequent predicted intestinal dissolution levels. All formulations sustained their intestinal dissolution level throughout the experiment, with no observed precipitation. Overall, the non-sink dissolution tests suggest the dispersions should enhance the bioavailability of etrumadenant relative to starting drug substance.

TABLE 8
Non-Sink Dissolution Data for Etrumadenant Dispersions

| | | | | Increase |
|---|---|---|---|---|
| | | | | in AUC |
| | Cₘₐₓ | | AUC₃₅₋₂₁₀ | over |
| | FaSSIF | C₂₁₀ | FaSSIF | API |
| Formulation | (μgA/mL) | (μgA/mL) | (min*μgA/mL) | AUC |
| 25:75 | 394.6 | 306.6 | 57300 | 7.2 |
| AB928:HPMCAS-M | | | | |
| SDI | | | | |
| 40:60 | 337.2 | 220.1 | 43400 | 5.4 |
| AB928:HPMCAS-M | | | | |
| SDI | | | | |
| 25:75 AB928:PVP-VA | 202.2 | 194.4 | 33900 | 4.2 |
| SDI | | | | |
| 25:75 | 258.8 | 162.2 | 32500 | 4.1 |
| AB928:HPMCAS-L | | | | |
| SDI | | | | |
| 40:60 AB928:PVP-VA | 191.6 | 172.4 | 31500 | 3.9 |
| SDI | | | | |
| 25:75 AB928:CAP | 205.6 | 165.0 | 30200 | 3.8 |
| SDI | | | | |
| 40:60 | 238.7 | 167.9 | 29900 | 3.7 |
| AB928:HPMCAS-L | | | | |
| SDI | | | | |
| 40:60 AB928:CAP | 216.7 | 98.8 | 26300 | 3.3 |
| SDI | | | | |
| AB928 starting | 155.0 | 37.9 | 8000 | NA |
| drug substance | | | | |

Cₘₐₓ FaSSIF = maximum drug concentration after transfer to FaSSIF
C₂₁₀ = drug concentration at 210 minutes, which is 180 minutes after transfer to FaSSIF
AUC₃₅₋₂₁₀ FaSSIF = area under the curve after transfer to FaSSIF from 35 to 210 minutes

To screen the physical and chemical stability of the etrumadenant dispersions, SDI powders were stressed for 4 and 12 weeks at 25° C. and relative humidity (RH) of 60% (25° C./60% RH) in an open high density polyethylene (HDPE) bottle without cap and cotton batting in neck of bottle (“open packaging”), 40° C./75% RH in open packing and 40° C./75% RH in closed packaging (i.e., double low density polyethylene (LDPE) bags, each goose-necked closed with a cable tie; 0.5 g silica gel desiccant added in between LDPE bags; and bags placed into and HDPE bottle). The SDI powders were evaluated for physical and chemical stability by appearance, amorphous character by XRPD, assay and related substances by HPLC, particle morphology by SEM, and presence of crystallinity by polarized light microscopy (PLM) (12 week only).

Table 9 summarizes appearance changes on stability. Notably, all SDIs demonstrated stability for up to 12 weeks in open-dish, accelerated stability conditions. PVP-VA formulations showed greater fusion of particles under high humidity conditions.

TABLE 9
Evaluation of Etrumadenant SDIs on Stability by Appearance

| | | Appearance | | |
|---|---|---|---|---|
| SDI | Storage | t = | t = | t = |
| Description | Condition | 0 | 4 weeks | 12 weeks |
| 25:75 | 25° C./60% | Off- | Off-White | Off-White |
| AB928:HPMCAS- | RH/OPEN | White | Powder | Powder |
| M | 40° C./75% | Powder | Off-White | Off-White |
| | RH/CLOSED | | Powder | Powder |
| | 40° C./75% | | Off-White | Off-White |
| | RH/OPEN | | Powder | Powder |
| 40:60 | 25° C./60% | Off- | Off-White | Off-White |
| AB928:HPMCAS- | RH/OPEN | White | Powder | Powder |
| M | 40° C./75% | Powder | Tan | * No |
| | RH/CLOSED | | powder | Sample |
| | 40° C./75% | | Tan | Tan |
| | RH/OPEN | | granules | Powder |
| 25:75 | 25° C./60% | Off- | Off-White | Off-White |
| AB928:PVP-VA | RH/OPEN | White | Powder | Powder |
| | 40° C./75% | Powder | Off-White | Amber |
| | RH/CLOSED | | Powder | Granules |
| | 40° C./75% | | Yellow- | Amber |
| | RH/OPEN | | Brown Glass | Glass |
| 40:60 | 25° C./60% | Off- | Off-White | Off-White |
| AB928:PVP-VA | RH/OPEN | White | Powder | Powder |
| | 40° C./75% | Powder | Amber | Off-White |
| | RH/CLOSED | | granules | Powder |
| | 40° C./75% | | Yellow/ | Amber |
| | RH/OPEN | | Brown Glass | Glass |

* Due to material limitations, there was no 12 week 40° C./75% RH sample for the 40:60 AB928:HPMCAS-M SDI.

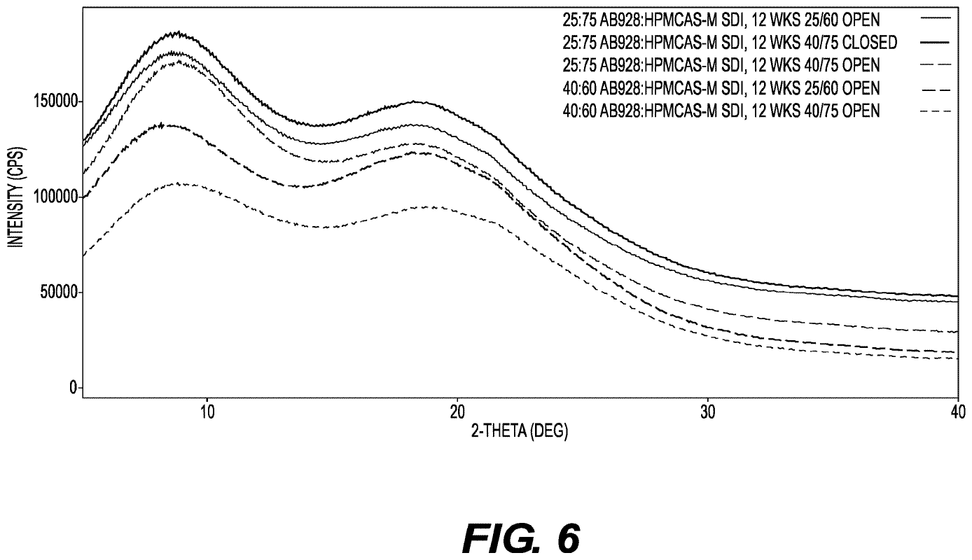

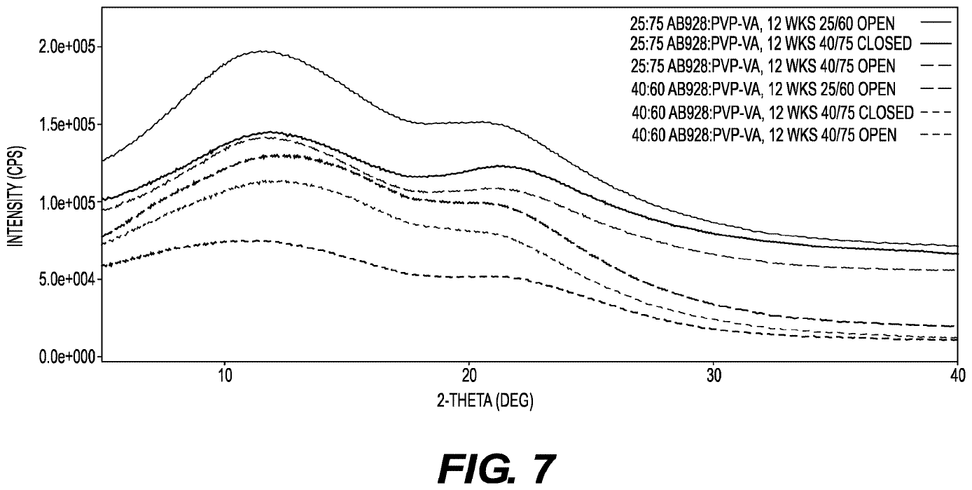

XRPD analysis of the SDI stability samples showed that all SDIs remained amorphous with no detectable crystalline material after 12 weeks (FIG. 6 and FIG. 7). 4-week diffractograms also showed an amorphous halo.

Surface morphology of the SDI particles was characterized using SEM. At 4 weeks a small amount of particle fusion was observed for the 40:60 AB928:HPMCAS-M SDI at 40° C./75% RH open and closed conditions. By 12 weeks, this fusion occurred to a greater extent. For the remaining AB928:HPMCAS-M SDI samples, typical SDI morphology apparent at t=0 was observed consisting of whole and collapsed spheres with smooth surfaces. No crystalline material was observed in any samples.

Fusion was also observed for the AB928:PVP-VA SDI samples analyzed by SEM at 12 weeks. SDI particles were fused into large blocks in samples of 25:75 AB928:PVP-VA SDI at 40° cn5% RH open and closed, and samples of 40:60 AB928:PVP-VA SDI at 40° C. M5% RH open. Slight fusion was apparent in individual SDI particles in samples of 40:60 AB928:PVP-VA SDI at 25° C./60% RH open and 40° cn5% RH closed. Samples of 25:75 AB928:PVP-VA SDI at 25° C./60% RH showed typical SDI morphology apparent at t=0. No crystalline material was observed in any samples.

Purity analysis of the SDI stability samples showed no change in related impurities relative to crystalline API in all SDI samples when stored at 2-8° C. for 4 weeks and 25° C./60% RH, 40° C./75% RH, open and closed for 4 and 12 weeks.

### Example 5: Exemplary Solid Dispersions of Etrumadenant

Four dispersions prepared with different ratios of starting drug substance to HPMCAS-M (25:75, 30:70, 35:65, 40:60) were prepared for stability testing. Each formulation was spray dried from acetone:water (95:05) as generally described in Table 10. Samples of the dispersions were obtained at the time of manufacturing (t=0) and after storage (t=1 month, 3 months, 6 months) at 25±2° C./60±5% RH in open and closed packaging, and 40±2° C./75±5% RH in open and closed packaging. Samples were characterized by a variety of analytical methods including powder X-ray diffraction (XRPD), modulated differential scanning calorimetry (MDSC), assay and impurity/related substances by high-performance liquid chromatography (HPLC), and non-sink dissolution. Samples obtained after 6 months on stability in open packaging were only analyzed for appearance and by MDSC. Descriptions of the analytical methods are provided in Example 4.

TABLE 10
Summary of Spray Drying Parameters

| Parameter | Value | |
|---|---|---|
| Spray Dryer | Buchi B290 | |
| Cyclone | High Efficiency | |
| Solvent | Acetone:Water (95:5) | |
| Batch Size, Active (A) | 6.25-10.0 | gA |
| Solution Composition | 10% | solids |
| Atomization Pressure | 28 | psi |
| Solution Feed Rate | ~20 | g/min |
| Inlet Temperature | 104-116° | C. |
| Outlet Temperature | 41-43° | C. |

| | |
|---|---|
| Secondary Drying | 18 hr at 40° C. |

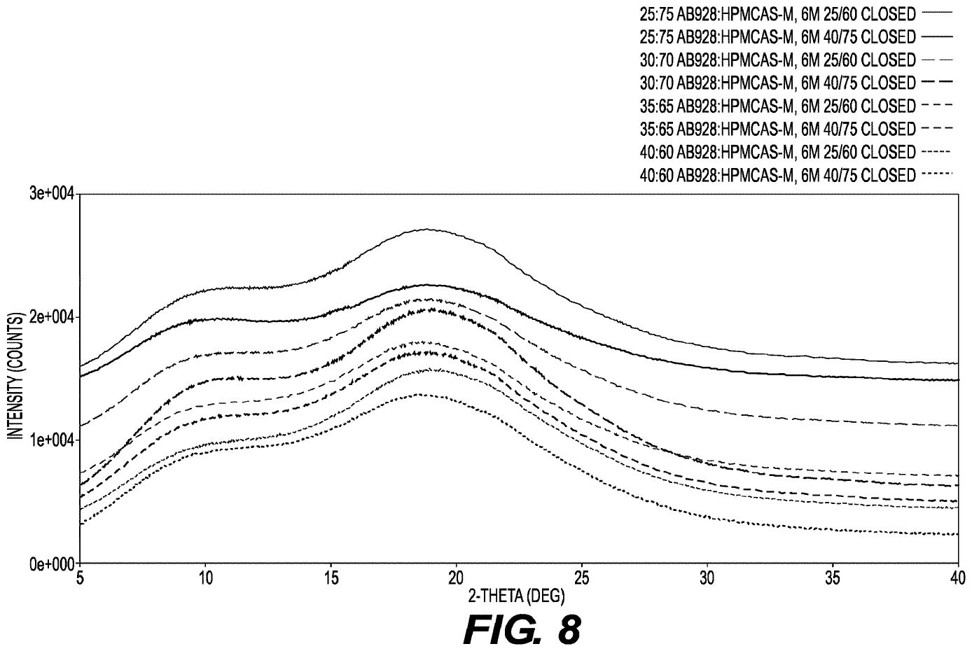

All samples remained an off-white powder after 6 months on stability and appeared amorphous by XRPD (FIG. 8). Assay and impurity analysis by HPLC showed that, after 6 months on stability assay, values of etrumadenant are near target (±10%), the impurity profile matches that of the starting drug substance, and there was no significant impurity growth after 6 months.

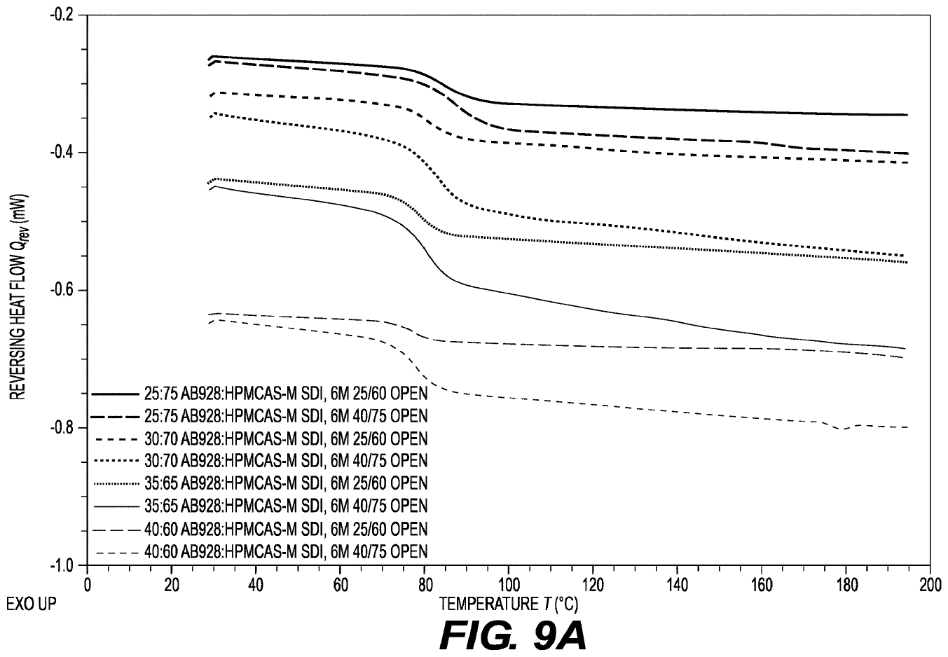

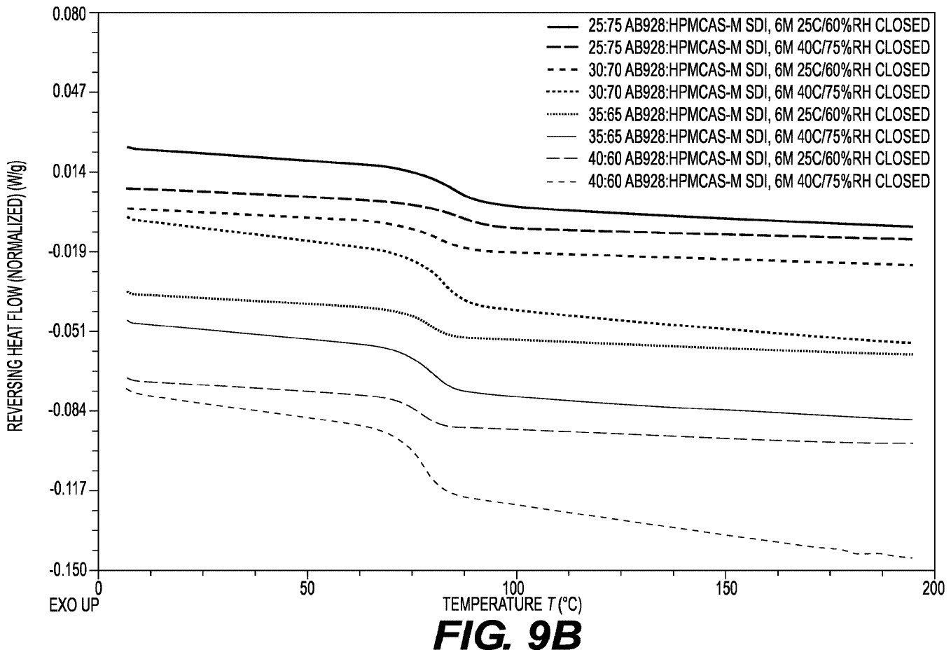

Samples analyzed by MDSC showed a single T_{g} (from about at all time points, including after 6 months on stability). An additional thermal event at ˜180° C. was detected after 3 weeks (open packaging) or 1 month (closed packaging) on stability for 40:60 AB928:HPMCAS-M SDIs by MDSC non-reversing. The event appeared to increase in magnitude during storage. No change in appearance was observed between 3 months and 6 months. An additional thermal event at ˜180° C. was also observed by MDSC non-reversing in 35:75 AB928:HPMCAS-M SDIs after 6 months on stability in open and closed packaging. The additional endothermic events observed at ˜180° C. suggested low level crystallization. T_{g} values are summarized in Table 11 and Table 12 (+ indicates a temperature of about 70° C. to about 90° C.; ND=not determined); representative MDSC curves are shown in FIG. 9.

TABLE 11
T_{g} values measured by MDSC

| | Stability | T_{g} (° C.) | | | |
|---|---|---|---|---|---|
| Sample Description | Condition | t = 0 | t = 1 M* | t = 3 M | t = 6 M |
| 25:75 AB928:HPMCAS-M SDI | 25° C./60% RH | + | + | + | + |
| | Open | | | | |
| 25:75 AB928:HPMCAS-M SDI | 40° C./75% RH | + | + | + | + |
| | Open | | | | |
| 30:70 AB928:HPMCAS-M SDI | 25° C./60% RH | + | + | + | + |
| | Open | | | | |
| 30:70 AB928:HPMCAS-M SDI | 40° C./75% RH | + | + | + | + |
| | Open | | | | |
| 35:65 AB928:HPMCAS-M SDI | 25° C./60% RH | + | + | + | + |
| | Open | | | | |
| 36:65 AB928:HPMCAS-M SDI | 40° C./75% RH | + | + | + | + |
| | Open | | | | |
| 40:60 AB928:HPMCAS-M SDI | 25° C./60% RH | + | + | + | + |
| | Open | | | | |
| 40:60 AB928:HPMCAS-M SDI | 40° C./75% RH | + | + | + | + |
| | Open | | | | |

*MDSC samples for 1 month on stability were pulled at 3 weeks for the open containers.

TABLE 12
T_{g} values measured by MDSC

| | Stability | T_{g} (° C.) | | | |
|---|---|---|---|---|---|
| Sample Description | Condition | t = 0 | t = 1 M | t = 3 M | t = 6 M |
| 25:75 AB928:HPMCAS-M SDI | 25° C./60% RH | + | ND | + | + |
| | Closed | | | | |
| 25:75 AB928:HPMCAS-M SDI | 40° C./75% RH | + | + | + | + |
| | Closed | | | | |
| 30:70 AB928:HPMCAS-M SDI | 25° C./60% RH | + | ND | + | + |
| | Closed | | | | |
| 30:70 AB928:HPMCAS-M SDI | 40° C./75% RH | + | + | + | + |
| | Closed | | | | |
| 35:65 AB928:HPMCAS-M SDI | 25° C./60% RH | + | ND | + | + |
| | Closed | | | | |
| 36:65 AB928:HPMCAS-M SDI | 40° C./75% RH | + | + | + | + |
| | Closed | | | | |
| 40:60 AB928:HPMCAS-M SDI | 25° C./60% RH | + | ND | + | + |
| | Closed | | | | |
| 40:60 AB928:HPMCAS-M SDI | 40° C./75% RH | + | + | + | + |
| | Open | | | | |

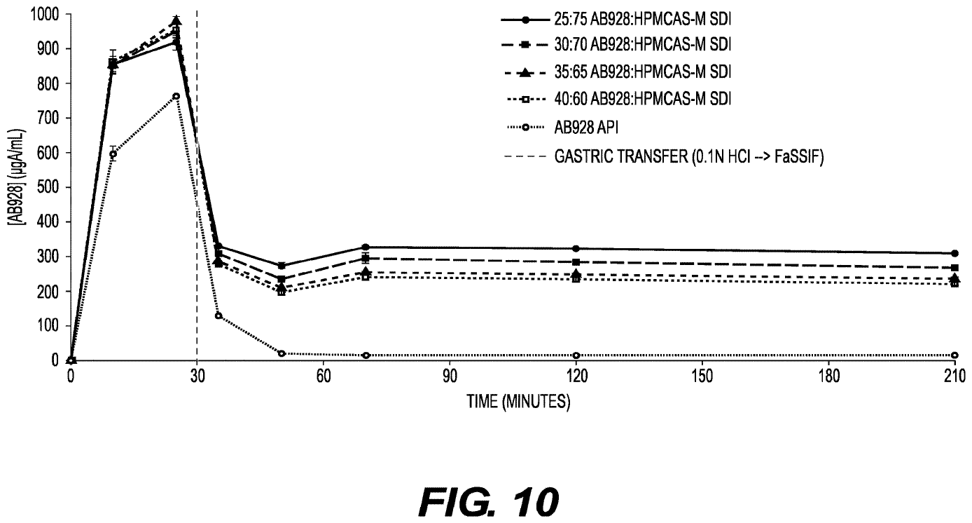

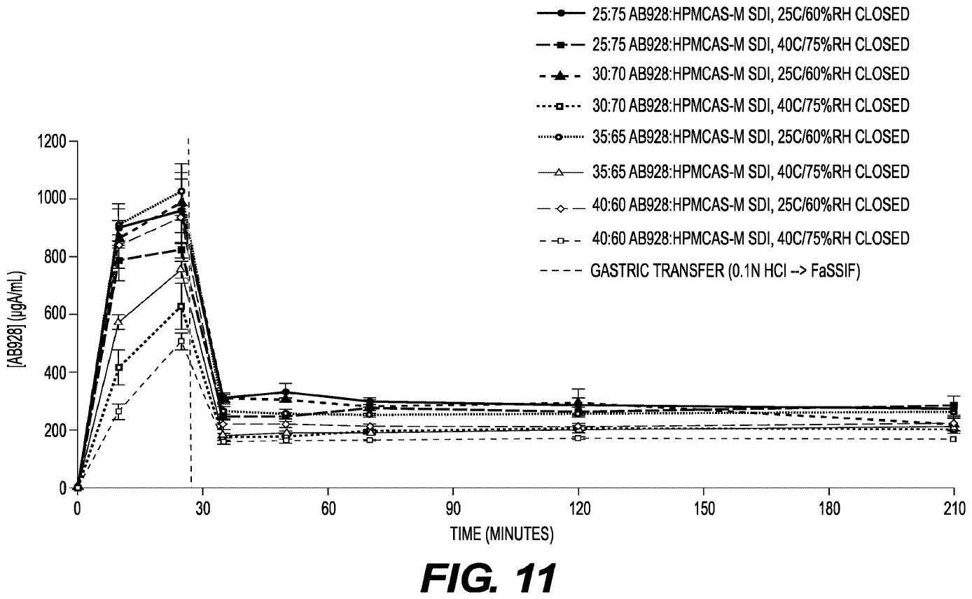

Non-sink dissolution testing at t=0 confirmed that formulating etrumadenant with HMPCAS-M as a solid dispersion improved etrumadenant's solubility in FaSSIF. AUC_{35-210 FaSSIF} decreased with increasing drug load. Samples were placed on stability in closed or open containers at 25° C. G60% RH and at 40° C./75% RH, and non-sink dissolution testing was again evaluated. Non-sink dissolution data are summarized in Tables 13 and 14; representative dissolution curves are shown in FIG. 10 and FIG. 11.

TABLE 13
Non-sink dissolution data

| | | Total Drug C_{maxFaSSIF} | | | AUC_{35-210 FaSSIF} | | | C₂₁₀ | | |
|---|---|---|---|---|---|---|---|---|---|---|
| | Stability | (μgA/mL) | | | (min*μgA/mL) | | | (μgA/mL) | | |
| SDI Description | Condition | t = 0 | t = 1 M | t = 3 M | t = 0 | t = 1 M | t = 3 M | t = 0 | t = 1 M | t = 3 M |
| 25:75 | 25° C./60% RH | 329.0 | 342.9 | 349.0 | 55400 | 57700 | 51700 | 309.8 | 321.6 | 278.4 |
| AB928:HPMCAS-M | Open | | | | | | | | | |
| 25:75 | 40° C./75% RH | 329.0 | 324.7 | 270.1 | 55400 | 55600 | 45800 | 309.8 | 312.0 | 253.2 |
| AB928:HPMCAS-M | Open | | | | | | | | | |
| 30:70 | 25° C./60% RH | 308.9 | 391.3 | 296.6 | 48800 | 63900 | 46100 | 268.3 | 391.3 | 250.2 |
| AB928:HPMCAS-M | Open | | | | | | | | | |
| 30:70 | 40° C./75% RH | 308.9 | 345.1 | 223.8 | 48800 | 50900 | 38400 | 268.3 | 345.1 | 216.1 |
| AB928:HPMCAS-M | Open | | | | | | | | | |
| 35:65 | 25° C./60% RH | 287.9 | 301.1 | 275.7 | 42900 | 48900 | 44000 | 236.2 | 265.9 | 239.7 |
| AB928:HPMCAS-M | Open | | | | | | | | | |
| 35:65 | 40° C./75% RH | 287.9 | 244.4 | 210.9 | 42900 | 39200 | 34700 | 236.2 | 244.4 | 210.9 |
| AB928:HPMCAS-M | Open | | | | | | | | | |
| 40:60 | 25° C./60% RH | 279.9 | 261.1 | 275.0 | 40500 | 42900 | 40300 | 222.4 | 254.3 | 225.9 |
| AB928:HPMCAS-M | Open | | | | | | | | | |
| 40:60 | 40° C./75% RH | 279.9 | 204.9 | 190.0 | 40500 | 33200 | 31300 | 222.4 | 204.9 | 190.0 |
| AB928:HPMCAS-M | Open | | | | | | | | | |
| AB928 starting | — | 129.9 | — | — | 3800 | — | — | 15.6 | — | — |
| drug substance | | | | | | | | | | |

TABLE 14
Non-sink dissolution data

| | | Total Drug C_{maxFaSSIF} | | | | AUC_{35-210 FaSSIF} | | | | C₂₁₀ | | | |
|---|---|---|---|---|---|---|---|---|---|---|---|---|---|
| SDI | Stability | (μgA/mL) | | | | (min*μgA/mL) | | | | (μgA/mL) | | | |
| Description | Condition | t = 0 | t = 1 M | t = 3 M | t = 6 M | t = 0 | t = 1 M | t = 3 M | t = 6 M | t = 0 | t = 1 M | t = 3 M | t = 6 M |
| 25:75 | 25° C./60% | 329.0 | ND | 428.3 | 329.9 | 55400 | ND | 69900 | 50800 | 309.8 | ND | 396.5 | 272.7 |
| AB928:HPMCAS-M | RH | | | | | | | | | | | | |
| | Closed | | | | | | | | | | | | |
| 25:75 | 40° C./75% | 329.0 | 338.1 | 406.3 | 282.4 | 55400 | 56400 | 66400 | 46800 | 309.8 | 309.6 | 359.9 | 282.4 |
| AB928:HPMCAS-M | RH | | | | | | | | | | | | |
| | Closed | | | | | | | | | | | | |
| 30:70 | 25° C./60% | 308.9 | ND | 339.5 | 309.8 | 48800 | ND | 56400 | 47700 | 268.3 | ND | 330.0 | 218.0 |
| AB928:HPMCAS-M | RH | | | | | | | | | | | | |
| | Closed | | | | | | | | | | | | |
| 30:70 | 40° C./75% | 308.9 | 318.7 | 306.5 | 202.6 | 48800 | 49800 | 51500 | 34400 | 268.3 | 274.4 | 292.6 | 201.6 |
| AB928:HPMCAS-M | RH | | | | | | | | | | | | |
| | Closed | | | | | | | | | | | | |
| 35:65 | 25° C./60% | 287.9 | ND | 298.1 | 263.8 | 42900 | ND | 45200 | 44900 | 236.2 | ND | 251.6 | 262.2 |
| AB928:HPMCAS-M | RH | | | | | | | | | | | | |
| | Closed | | | | | | | | | | | | |
| 35:65 | 40° C./75% | 287.9 | 282.3 | 290.8 | 209.2 | 42900 | 44800 | 45600 | 35100 | 236.2 | 246.8 | 250.3 | 209.2 |
| AB928:HPMCAS-M | RH | | | | | | | | | | | | |
| | Closed | | | | | | | | | | | | |
| 40:60 | 25° C./60% | 279.9 | ND | 236.1 | 222.7 | 40500 | ND | 39400 | 37500 | 222.4 | ND | 223.8 | 222.7 |
| AB928:HPMCAS-M | RH | | | | | | | | | | | | |
| | Closed | | | | | | | | | | | | |
| 40:60 | 40° C./75% | 279.9 | 268.1 | 182.0 | 171.7 | 40500 | 40200 | 31500 | 29300 | 222.4 | 234.5 | 181.6 | 167.4 |
| AB928:HPMCAS-M | RH | | | | | | | | | | | | |
| | Closed | | | | | | | | | | | | |
| Etrumadenant | — | 129.9 | — | — | — | 3800 | — | — | — | 15.5 | — | — | — |

ND = not determined

### Example 6: Exemplary Tablets Comprising Solid Dispersions of Etrumadenant

Four dispersions prepared with different ratios of starting drug substance to polymer were prepared for tableting: 25:75 AB928:HPMCAS-M, 40:60 AB928:HPMCAS-M, 25:7.5:67.5 AB928:TPGS:PVP-VA, and 40:7.5:52.5 AB928:TPGS:PVP-VA. TPGS (tocofersolan) was incorporated into the two PVP-VA SDIs at 7.5% (w/w) to evaluate the effect of a surfactant on dissolution performance. Each formulation was spray dried from neat acetone according to the parameters summarized in Table 15. The resulting SDI powders were then characterized by a variety of methods, including XRPD, SEM, MDSC, and in vitro non-sink dissolution. Descriptions of the analytical methods are provided in Example 4.

TABLE 15
Summary of Spray Drying Parameters

| Parameter | Value | |
|---|---|---|
| Spray Dryer | Buchi B290 | |
| Cyclone | Standard | |
| Solvent | Acetone | |
| Batch Size, Active (A) | 35 gA, 55 gA | |
| Solution Composition | 8-10% solids (w/w) | |
| Atomization Pressure | 28 | psi |
| Solution Feed Rate | ~18-19 | g/min |
| Inlet Temperature | 80-87° | C. |
| Outlet Temperature | 44-46° | C. |

| | |
|---|---|
| Secondary Drying | 24 hours at 40° C. |

Thermal analysis done by MDSC showed that all four of the dispersions had a single T_{g}, indicating an intimately mixed amorphous solid dispersion with good homogeneity. T_{g} values were from about 72° C. to about 83° C. Characterization by XRPD indicated that the SDIs were amorphous dispersions, and no crystalline peaks were observed in the SDI diffractograms. Surface morphology of the SDI particles, characterized by SEM, showed typical SDI morphology consisting of whole and collapsed spheres with smooth surfaces. No crystalline material was observed in any sample. The dissolution performance of the SDIs was tested in the non-sink dissolution test. All SDIs showed similar dissolution performance compared to the SDIs described in Example 4, with the 25% AB928:HPMCAS-M formulation performing the best. Introduction of TPGS at 7.5 wt % into the PVP-VA SDIs did not yield significant dissolution increases in vitro.

Immediate release tablets comprising the four dispersions were then developed. Due to poor flow properties of the dispersions, efforts were made to develop a dry granule comprising the SDI.

For the HPMCAS SDIs, a 1:1 microcrystalline cellulose (MCC):mannitol ratio was used, as it is contemplated that a mixture of brittle and plastic fillers may help improve tablet mechanical properties. Croscarmellose sodium (Ac-Di-Sol) was selected as the super disintegrant as it is contemplated that its use may achieve acceptable disintegration times. Colloidal silica (Cab-O-Sil) was used as a glidant as it is contemplated to improve flowability, which may be useful for scale-up work. Sodium stearyl fumarate (SSF) was selected as the lubricant as it is contemplated to help reduce sticking of formulation on process equipment.

For the PVP-VA SDIs, crospovidone (Kollidon CL) was added as a superdisintegrant, and mesoporous silica (Parteck SLC and Syloid XDP 3150) were added as disintegration aids; it is contemplated that these components could reduce disintegration time. Additionally, silicified microcrystalline cellulose (Prosolv SMCC 90) was used in place of microcrystalline cellulose and colloidal silica. After dry granulation, additional excipients were added prior to tableting. Extragranular excipients were selected as they were contemplated to reduce the risk of over-compressing the formulation during tableting.

TABLE 16
50 mgA and 100 mgA Tablet Formulations

| | AB928:HPMCAS-M SDIs | | | AB928:TPGS:PVP-VA SDIs | | |
|---|---|---|---|---|---|---|
| | | mg/tablet | mg/tablet | | mg/tablet | mg/tablet |
| Material | (% w/w) | (50 mgA) | (100 mgA) | (% w/w) | (50 mgA) | (100 mgA) |
| Intra-Granular | | | | | | |
| 25:75 AB928:HPMCAS-M SDI | 40.0 | 200.0 | — | — | — | — |
| 40:60 AB928:HPMCAS-M SDI | | — | 250.00 | | — | — |
| 25:7.5:67.5 AB928:TPGS:PVP- | — | | | 40.0 | 200.0 | — |
| VA SDI | | | | | | |
| 40:7.5:52.5 AB928:TPGS:PVP- | | | | | — | 250.00 |
| VA SDI | | | | | | |
| Microcrystalline cellulose | 20.0 | 100.0 | 125.00 | — | — | — |
| (Avicel PH-105) | | | | | | |
| Mannitol | 20.0 | 100.0 | 125.00 | — | — | — |
| Croscarmellose sodium | 3.5 | 17.5 | 21.88 | — | — | — |
| Colloidal silica | 0.5 | 2.5 | 3.12 | — | — | — |
| Silicified microcrystalline | — | — | — | 14.0 | 70.0 | 128.13 |
| cellulose (SMCC 90) | | | | | | |
| Mesoporous silica (Partek SLC) | — | — | — | 20.5 | 102.5 | 31.25 |
| Crospovidone (Kollidon CL) | — | — | — | 5.0 | 2.5 | 3.12 |
| Sodium stearyl fumarate | 0.5 | 2.5 | 3.13 | 0.5 | | |
| Extra-Granular | | | | | | |
| Microcrystalline cellulose | 6.5 | 32.5 | 40.63 | — | — | — |
| Mannitol | 6.5 | 32.5 | 40.62 | — | — | — |
| Croscarmellose sodium | 1.5 | 7.5 | 9.37 | — | — | — |
| Colloidal silica | 0.5 | 2.5 | 3.13 | — | — | — |
| Silicified microcrystalline | — | — | — | 5.0 | 27.5 | 34.38 |
| cellulose (SMCC 90) | | | | | | |
| Mesoporous silica (Syloid XDP | — | — | — | 9.0 | 45.0 | 56.25 |
| 3150) | | | | | | |
| Crospovidone (Kollidon CL) | — | — | — | 5.0 | 25.0 | 31.25 |
| Sodium stearyl fumarate | 0.5 | 2.5 | 3.12 | 0.5 | 2.6 | 3.12 |
| Core Tablet Total | 100.0 | 500.0 | 625.00 | 100.0 | 500.0 | 625.00 |

Tablets were prepared as follows. SDI was blended with intragranular components in a suitable blender. The blend was dc-lumped using a suitable conical mill and then subject to roller compaction and milling. Using a roller compactor with an oscillating mill equipped with suitable screen (25 mesh), the blend was first compacted into ribbons (targeting a solid fraction of about 0.7), which were then milled into granules. Extragranular components were then blended with the granules and the mixture subject to tableting. Tablets were compressed at the target mass using a rotary tablet press. Throughout the compression operation, individual tablet weight, the average weight of 10 tablets, tablet hardness and thickness were monitored at predetermined manufacturing process intervals.

Tabletability, compressibility, compactability, and disintegration profiles were generated for all formulations using 0.4062″ and 0.5000″ standard round convex (SRC) tooling for the 50 mgA and 100 mgA tablets, respectively. Compression profiles were generated over the range of 100-200 MPa.

Disintegration was evaluated per USP <701>“Disintegration” utilizing a Varian VK-100 disintegration apparatus. The apparatus consists of a 1000 mL low-form beaker and basket-rack assembly with six open-ended transparent tubes. The beaker contained 750 mL of RO1 water and was maintained at a temperature of 37° C. (±2° C.). The basket was fully submerged at a frequency of 29-32 cycles per minute and tablet disintegration time was recorded when the last visible tablet materials passed through the basket.

Tablet tensile strength was calculated based on the equation below, which applies to Standard round concave (SRC) tablets: Where P=fracture load, D=tablet width, t=tablet thickness, W=band thickness.

$$Tablet Tensile Strength ( SRC ) = \frac{10 P}{π {{D}^{2} ( 2.84 \frac{t}{D} - 0.126 \frac{t}{W} + 3.15 \frac{W}{D} + 0.01 )}^{2}}$$

Tablet hardness was tested per USP <1217>“Tablet Breaking Force” utilizing a Natoli Hardness Tester (S/N 1403029). Tablet thickness and weight were measured prior to assessing the tablet break force as it is a destructive process. Tablets were placed in the automated breaking apparatus and tablet hardness was measured in kilopond (kP) or kilogram-force.

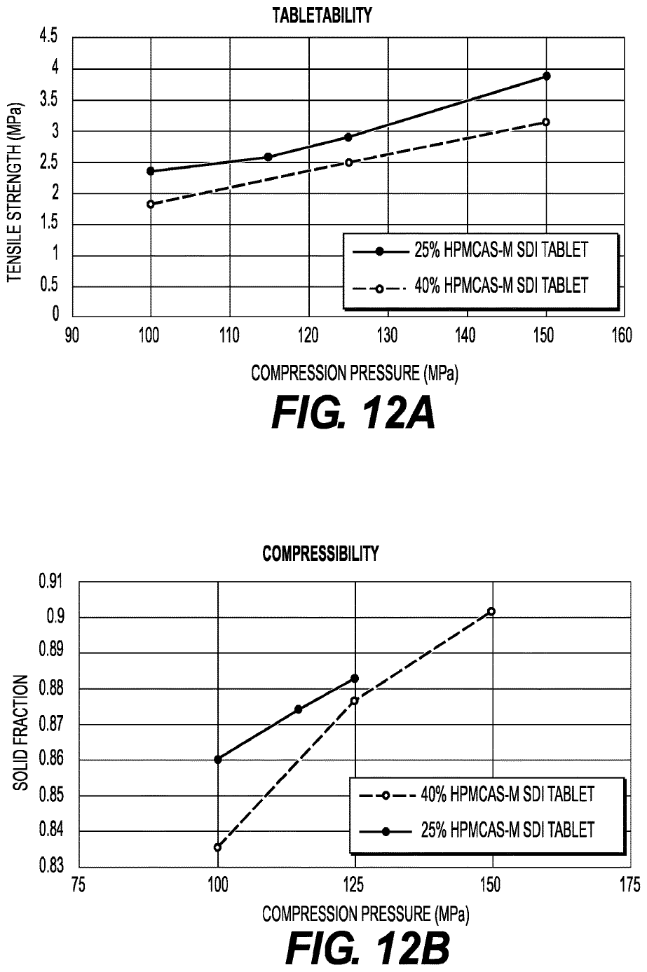

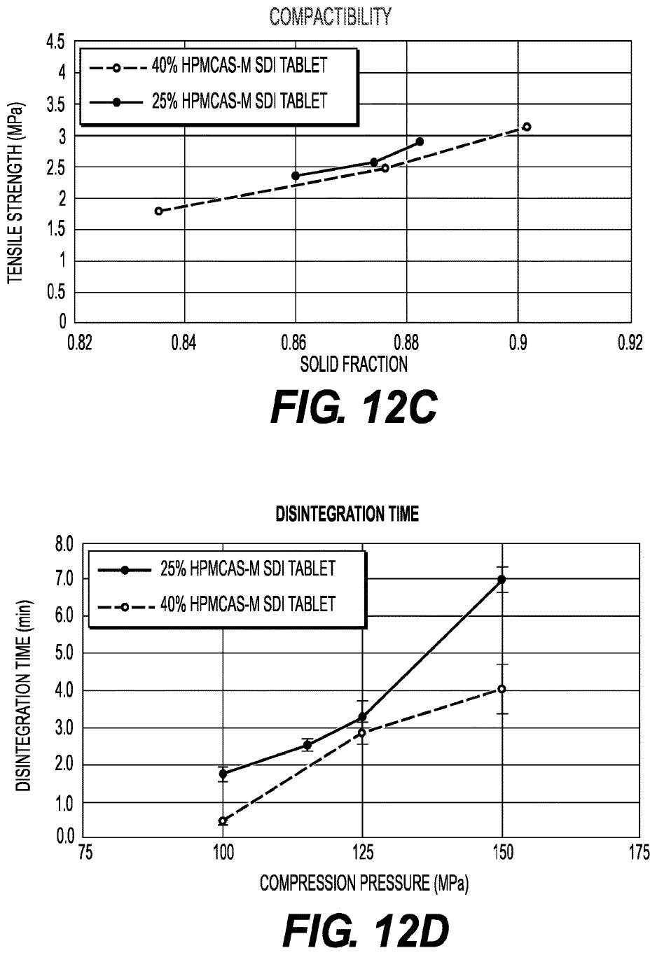

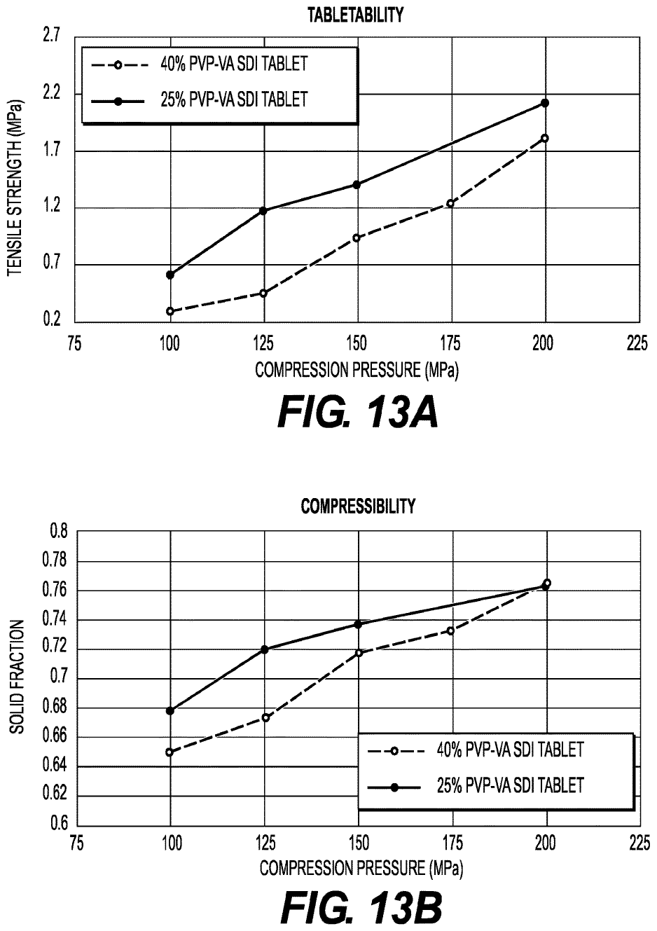

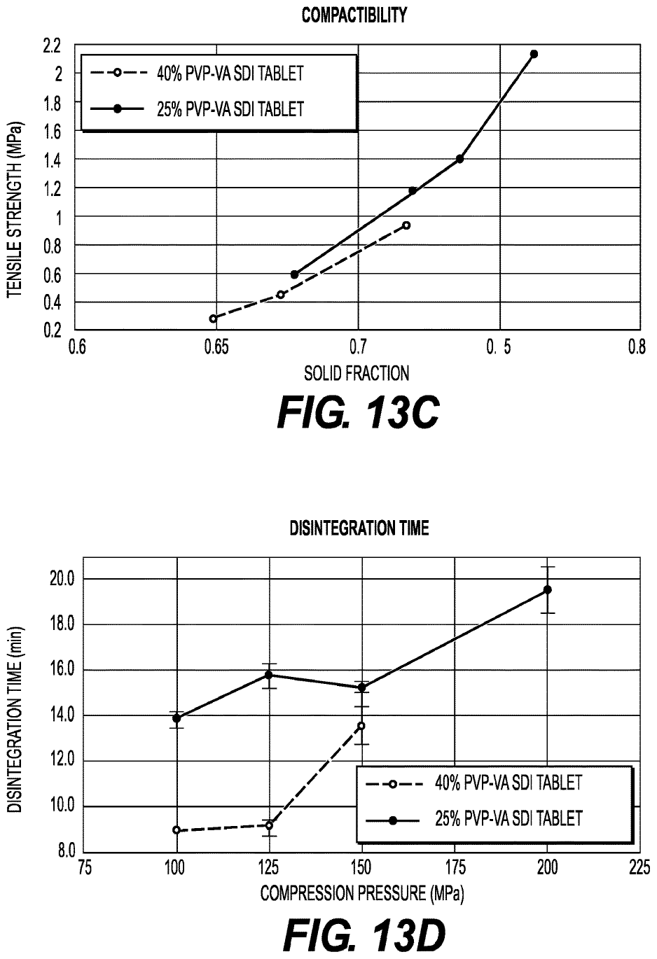

HPMCAS-M SDI tablets showed increased tabletability compared to PVP-VA SDI tablets and achieved higher tensile strength at given compression pressure. PVP-VA SDI tablets showed longer disintegration times than HPMCAS-M SDI tablets. See FIG. 12 and FIG. 13.

The four tablet formulations were also characterized for assay and related substances/impurity by HPLC and by non-sink dissolution, as described in Example 4, and water content by Karl Fischer coulometric titration using an oven drying method. Water content values for the tablets were consistent across both drug loadings for each polymer formulation. PVP-VA SDI tablets contained a higher water content, it is contemplated, due to the increased hygroscopicity of PVP-VA polymer compared to HPMCAS-M. Assay and related substances/impurities analysis of the tablets by HPLC showed similar total related substances compared to the parent SDIs or as-received crystalline etrumadenant, indicating that no chemical degradation occurred during the spray drying or tablet manufacturing processes.

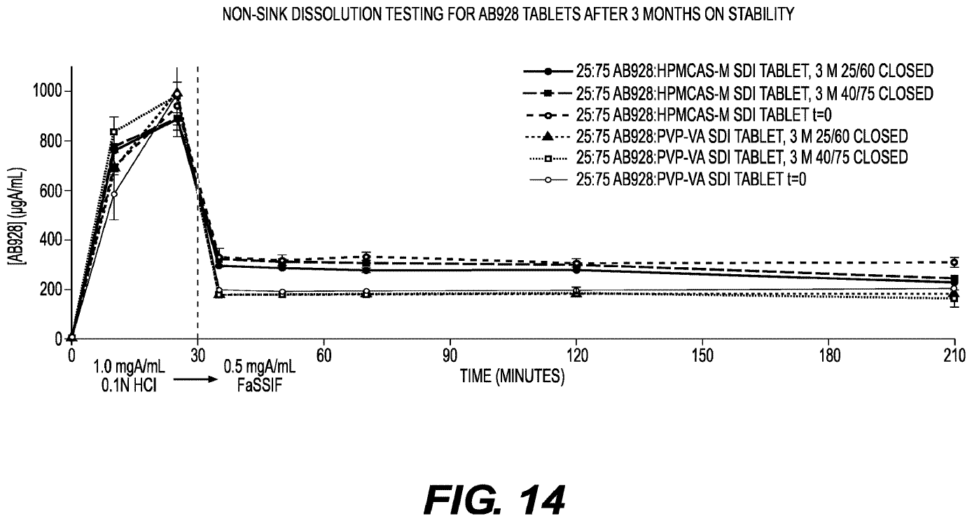

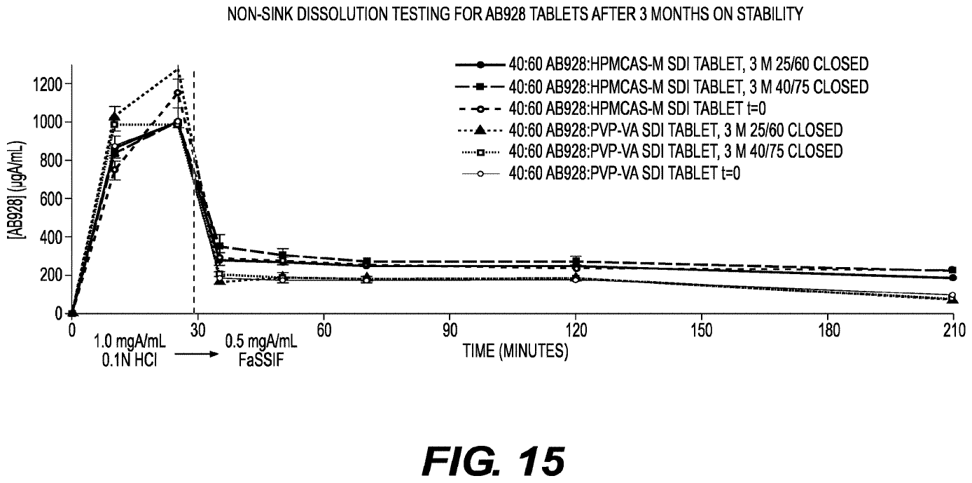

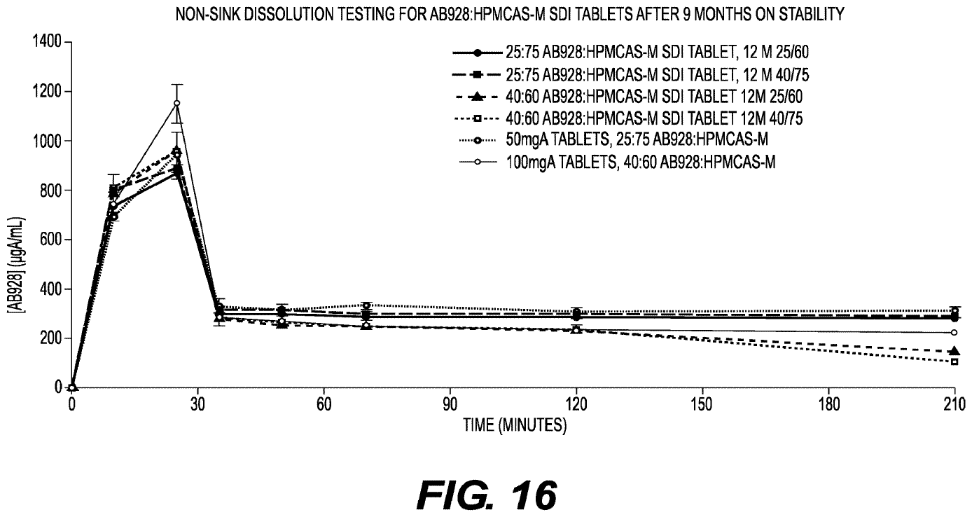

The dissolution performance of the tablets was tested in the non-sink dissolution test. Dissolution performance of the tablets was also evaluated under accelerated stability conditions, 25° C./60% RH and 40° C./75% RH, after 1 and 3 months of storage in closed packaging. Release testing results (described above) were utilized for the t=0 timepoint. Representative data are shown in FIG. 14 and FIG. 15. Non-sink dissolution testing of AB928:HPMCAS-M SDI tablets also occurred after the tablets were on stability for 6, 9, and 12 months. See FIG. 16.

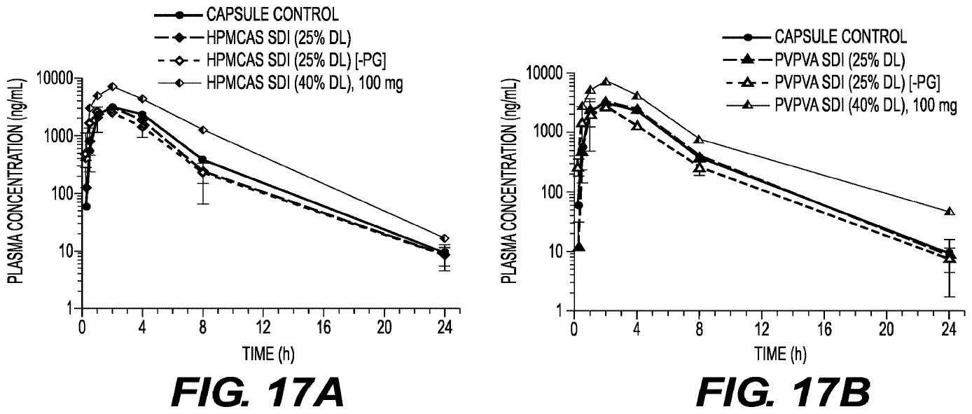

Pharmacokinetics (PK) of SDI tablets, as compared to a capsule control, were evaluated in male beagle dogs fasted overnight prior to study drug administration. Whole blood samples were collected pre-dose, and 0.25, 0.5, 1, 2, 4, 8, and 24 hours following administration (PO) of a single dose of study drug. PK of a crystalline AB928 tablet, as described in Table 2, was also evaluated under similar conditions. Formulations tested and PK data are summarized in Table 17 and Table 18 (Terminal_T½=terminal elimination half-life; MRTINF=mean residence time from time 0 to infinity; other parameters are as described herein). Representative data are shown in FIG. 17.

TABLE 17

| | | | | | AUClast | AUCINF | |
|---|---|---|---|---|---|---|---|
| | Dose | Terminal_T½ | Tmax | Cmax | (hr*ng/ | (hr*ng/ | MRTINF |
| Formulation | (mg) | (hr) | (hr) | (ng/mL) | mL) | mL) | (hr) |
| Crystalline | 50 | 2.61 ± 0.42 | 2 ± 0 | 1420 ± 452 | 6400 ± 1970 | 6480 ± 1890 | 4.68 ± 1.76 |
| AB928 | | | | | | | |
| Tablet | | | | | | | |

TABLE 18

| | | | | | AUClast | AUCINF | |
|---|---|---|---|---|---|---|---|
| | Dose | Terminal_T½ | Tmax | Cmax | (hr*ng/ | (hr*ng/ | MRTINF |
| Formulation | (mg) | (hr) | (hr) | (ng/mL) | mL) | mL) | (hr) |
| Capsule | 50 | 2.64 ± | 1.75 ± | 3240 ± | 17500 ± | 17500 ± | 4.13 ± |
| Control | | 0.18 | 0.50 | 616 | 2850 | 2840 | 0.25 |
| HPMCAS | 50 | 2.75 ± | 2.00 ± | 3030 ± | 14400 ± | 14400 ± | 3.79 ± |
| SDD | | 0.18 | 0.00 | 241 | 2260 | 2280 | 0.38 |
| (25% DL) | | | | | | | |
| HPMCAS | 50 | 3.53 ± | 1.25 ± | 2720 ± | 13100 ± | 13100 ± | 3.56 ± |
| SDD | | 0.64 | 0.50 | 413 | 4170 | 4170 | 0.48 |
| (25% DL) - | | | | | | | |
| PG | | | | | | | |
| HPMCAS | 50 | 2.45 ± | 1.50 ± | 2800 ± | 13200 ± | 14700 ± | 4.17 ± |
| SDD | | 0.39 | 0.58 | 299 | 4150 | 1790 | 0.46 |
| (40% DL) | | | | | | | |
| HPMCAS | 100 | 2.47 ± | 1.75 ± | 7110 ± | 41000 ± | 41000 ± | 4.44 ± |
| SDD | | 0.20 | 0.50 | 985 | 5400 | 5410 | 0.61 |
| (40% DL) | | | | | | | |
| PVPVA | 50 | 2.42 ± | 2.00 ± | 3400 ± | 17200 ± | 17500 ± | 3.96 ± |
| SDD | | 0.68 | 0.00 | 408 | 1150 | 1290 | 0.45 |
| (25% DL) | | | | | | | |
| PVPVA | 50 | 2.62 ± | 1.75 ± | 2880 ± | 12400 ± | 12600 ± | 3.74 ± |
| SDD | | 0.87 | 0.50 | 683 | 2080 | 2050 | 0.77 |
| (25% DL) - | | | | | | | |
| PG | | | | | | | |
| PVPVA | 50 | 2.05 ± | 1.50 ± | 3580 ± | 16200 ± | 16300 ± | 3.63 ± |
| SDD | | 0.50 | 0.58 | 592 | 3590 | 3460 | 0.38 |
| (40% DL) | | | | | | | |
| PVPVA | 100 | 3.27 ± | 1.63 ± | 7890 ± | 36700 ± | 36900 ± | 4.12 ± |
| SDD | | 0.33 | 0.75 | 1090 | 7600 | 7680 | 0.54 |
| (40% DL) | | | | | | | |

Capsule control formulation is described in Table 3.
DL = drug loading

### Example 7: Exemplary Tablets Comprising Solid Dispersions of Etrumadenant

25:75 AB928:HPMCAS-M SDI tablets were prepared as generally described in Example 6 with different intragranular excipients (Avicel PH-105, Partek M 100, FlowLac 90, Emcompress, Avicel DG, Nisso HPC SSL SFP) according to Table 19. During dry granulation, the target solid ribbon fraction was 0.6 to 0.7. For tableting, compression profiles were generated over the range of 50-200 MPa. Weight, thickness, hardness and disintegration of the resulting tablets were evaluated, as described in Example 6.

TABLE 19
Exemplary 25:75 AB928:HPMCAS-M SDI Tablet.

| | | Unit Composition (wt %) | | | | | | | |
|---|---|---|---|---|---|---|---|---|---|
| Component | Function | F1 | F2 | F3 | F4 | F5 | F1.1 | F1.2 | F4.1 |
| SDI | Drug | 60.0% | 60.0% | 60.0% | 60.0% | 60.0% | 53.33% | 53.33% | 53.33% |
| | Carrier | | | | | | | | |
| MCC | Filler | 16.5% | 16.5% | 16.5% | — | 14.0% | 14.33% | 9.55% | — |
| Mannitol | Filler | 16.5% | — | — | — | — | 14.34% | 19.12% | — |
| LM | Filler | — | 16.5% | — | — | — | — | — | 29.67% |
| DCP | Filler | — | — | 16.5% | | | | | |
| DCP/MCC | Filler | — | — | | 33.0% | | | | |
| HPC | Binder | — | — | | | 5.0% | | | |
| Ac-Di-Sol | Super | 3.5% | 3.5% | 3.5% | 3.5% | 3.5% | 4.00% | 4.00% | 3.50% |
| | Disintegrant | | | | | | | | |
| Cab-O-Sil | Glidant | 0.5% | 0.5% | 0.5% | 0.5% | 0.5% | 0.5% | 0.5% | 0.5% |
| SSF | Lubricant | 0.5% | 0.5% | 0.5% | 0.5% | 0.5% | 0.5% | 0.5% | 0.5% |
| Intragranular Total | | 97.5% | 97.5% | 97.5% | 97.5% | 97.5% | 87.0% | 87.0% | 87.0% |
| Ac-Di-Sol | Super | 1.5% | 1.5% | 1.5% | 1.5% | 1.5% | 2.00% | 2.00% | 2.00% |
| | Disintegrant | | | | | | | | |
| Cab-O-Sil | Glidant | 0.5% | 0.5% | 0.5% | 0.5% | 0.5% | 0.5% | 0.5% | 0.5% |
| PRUV ® | Lubricant | 0.5% | 0.5% | 0.5% | 0.5% | 0.5% | 0.5% | 0.5% | 0.5% |
| MCC | Filler | — | — | — | — | — | 5.00% | 3.33% | 5.00% |
| Mannitol | Filler | — | — | — | — | — | 5.00% | 6.67% | 5.00% |

| | | | | | | | | |
|---|---|---|---|---|---|---|---|---|
| Extragranular Total | 2.5% | 2.5% | 2.5% | 2.5% | 2.5% | 13.00% | 13.00% | 12.50% |
| Total | 100.0% | 100.0% | 100.0% | 100.0% | 100.0% | 100.00% | 100.00% | 100.00% |
| Tablet Dose (mg) | 100 | 100 | 100 | 100 | 100 | 100 | 100 | 100 |
| Tablet Weight (mg) | 667 | 667 | 667 | 667 | 667 | 750 | 750 | 750 |

DCP = dicalcium phosphate
HPC = hydroxypropylcellulose
LM = lactose monohydrate
MCC = microcrystalline cellulose
SSF = sodium stearyl fumarate

A first round of tablets, F1-F5, were initially developed. All formulations showed good weight, thickness, breaking force, tabletability and compressibility. Disintegration time for F1-F5 increased drastically at >150 MPa, indicating that achieving a robust disintegration profile was a significant technical hurdle.

A second round of tablets, F1.1, F1.2, and F4.1, were then developed to implement various approaches to reduce disintegration time (e.g., decrease SDI content, increase disintegrant, increase extragranular filler (e.g., Avicel PH-200, Partek M 200)). All formulations again showed good weight, thickness, breaking force, tabletability and compressibility. Notably, all formulations also achieved robust disintegration profiles.

### Example 8: Exemplary Tablets Comprising Solid Dispersions of Etrumadenant

Etrumadenant film-coated tablets were manufactured to contain 75 mg etrumadenant. Tablets were prepared as follows. SDI was blended with intragranular components in a suitable blender. The blend was de-lumped using a suitable conical mill and then subject to roller compaction and milling. Using a roller compactor with an oscillating mill equipped with suitable screen, the blend was first compacted into ribbons, which were then milled into granules. Extragranular components were then blended with the granules and the mixture subject to tableting. Tablets were compressed at the target mass using a rotary tablet press, and then film-coated. Throughout the compression operation, individual tablet weight, the average weight of 10 tablets, tablet hardness and thickness were monitored at predetermined manufacturing process intervals. Tablet composition is provided in Table 20.

TABLE 20
Etrumadenant Tablet Composition

| | |
|---|---|
| Material | Composition (% w/w) |

Intra-Granular

| | |
|---|---|
| 25:75 AB928:HPMCAS-M | 40.0^{a} |
| SDI | |
| Microcrystalline cellulose | 20.0^{a} |
| Mannitol | 20.0 |
| Croscarmellose sodium | 3.5 |
| Colloidal silica | 0.5 |
| Sodium stearyl fumarate | 0.5 |

Extra-Granular

| | |
|---|---|
| Microcrystalline cellulose | 6.5 |
| Mannitol | 6.5 |
| Croscarmellose sodium | 1.5 |
| Colloidal silica | 0.5 |
| Sodium stearyl fumarate | 0.5 |
| Core Tablet Total | 100.0 |

Film-Coating

| | |
|---|---|
| Opadry ® White | 4.0 |
| Purified Water | —^{b} |
| Total | 104.0 |

^{a}SDI is adjusted for potency with concomitant adjustment of microcrystalline cellulose;
^{b}Removed during the manufacture

Tablets were packaged in high-density polyethylene (HDPE) bottles containing desiccant and closed with a foil heat induction seal and a polypropylene (PP) child-resistant closure, and then placed on stability at 25° C./60% relative humidity and 40° C./75% relative humidity.

Tablets were sampled over time and tested for appearance, assay and impurities by HPLC, water content, and dissolution. Assay was determined via a reverse-phase gradient HPLC method. The impurities (and degradation products of etrumadenant) were determined via a reverse-phase gradient HPLC method. Impurities were identified only by HPLC relative retention time (RRT); these RRTs were approximate due to variations in HPLC analysis from time to time. The water content in etrumadenant was determined via Karl Fischer coulometric titration using an oven drying method according to USP <921>. The same HPLC method is for identification, assay and related substances, and the parameters are summarized in Table 21 and dissolution method in Table 22.

TABLE 21

| Parameter | Value | | |
|---|---|---|---|
| Column | ACE Excel 3 C18-PFP, 150 × 4.6 mm, 3 μm | | |
| Mobile Phase A | 20 mM Ammonium Carbonate Buffer, pH 6.2 | | |
| Mobile Phase B | Methanol | | |
| Program Type | Time(min) | % MPA | % MPB |
| | 0 | 95 | 5 |
| | 12.0 | 50 | 50 |
| | 32.0 | 30 | 70 |
| | 38.0 | 5 | 95 |
| | 50.0 | 5 | 95 |
| | 50.5 | 95 | 5 |
| | 60.0 | 95 | 5 |

| | |
|---|---|
| Flow Rate | 0.8 mL/min |
| Column Temperature | 30° C. |
| Sample Temperature | Ambient |
| Injection Volume | 5 μL |
| Detection Wavelength | UV @ 268 nm |
| Run Time | 60 min |

TABLE 22
Dissolution method for Etrumadenant Film-Coated Tablets
Dissolution Condition

| System | USP Apparatus II (Paddle) | |
|---|---|---|
| Medium | 0.1% sodium dodecyl sulfate in 0.01N HCl, | |
| | pH 2.0 | |
| Medium volume | 900 | mL |
| Medium temperature | 27 ± 0.5° | C. |
| Rotation speed | 75 | rpm |
| Sampling time points | 5, 10, 15, 20, 45, 60 and infinity (250 rpm) | |
| Sampling volume | 10 | mL |

| | |
|---|---|
| Medium replacement | No |

HPLC Conditions

| Column | Thermo Hypersil Gold, 3 μm, 100 × 4.6 mm or | |
|---|---|---|
| | equivalent | |
| Mobile phase | 0.1% phosphoric acid 70:30 water:ACN (v:v) | |
| Program type | Isocratic | |
| Flow rate | 1.5 | mL/min |
| Column temperature | 40° | C. |
| Sample temperature | Ambient | |
| Injection volume | 5 | μL |
| Need wash | Diluent | |
| Detection | UV @ 254 nm | |
| Run time | 5 | min |

There were no significant changes in appearance, assay, impurities, and dissolution after 12 months at long-term 25° C.±2° C./60%±5% RH and 6 months at accelerated 40° C.±2° C./75% t 5% RH storage conditions. There was gradual increase over time for the water content under both conditions.

T=0 data correspond to lot release results.

TABLE 23

| Test | T = 0 | T = 2 M | T = 3 M | T = 6 M | T = 9 M | T = 12 M |
|---|---|---|---|---|---|---|
| Appearance | conforms | conforms | conforms | conforms | conforms | conforms |
| Assay (% | 100.8 | 99.9 | 100.2 | 99.6 | 100.9 | 105.1 |
| Label Claim) | | | | | | |
| Total | 0.21 | 0.21 | 0.21 | 0.23 | 0.20 | 0.20 |
| Impurities | | | | | | |
| (% a/a) | | | | | | |
| Water | 1.20 | 1.47 | 1.40 | 1.82 | 1.77 | 1.45 |
| content (% | | | | | | |
| w/w) | | | | | | |
| Dissolution | 102 | 102 | 100 | 99 | 98 | 99 |
| (% Dissolved | | | | | | |
| at 45 min) | | | | | | |

### Example 9: Exemplary Tablets Comprising Solid Dispersions of Etrumadenant

Etrumadenant tablets were manufactured to contain 50 mg or 75 mg etrumadenant. Tablets were prepared as follows. SDI was blended with intragranular components in a suitable blender. The blend was de-lumped using a suitable conical mill and then subject to roller compaction and milling. Using a roller compactor with an oscillating mill equipped with suitable screen, the blend was first compacted into ribbons, which were then milled into granules. Extragranular components were then blended with the granules, and the mixture subject to tableting. Tablets were compressed at the target mass using either a manual or rotary tablet press. Tablet composition is provided in Table 24.

TABLE 24
Etrumadenant Tablet Composition

| | |
|---|---|
| Material | Composition (% w/w) |

Intra-Granular

| | |
|---|---|
| 25:75 AB928:HPMCAS SDI | 40 |
| Microcrystalline cellulose | 26 |
| Mannitol | 18 |
| Croscarmellose sodium | 5 |
| Magnesium Stearate | 0.5 |

Extra-Granular

| | |
|---|---|
| Microcrystalline cellulose | 10 |
| Magnesium Stearate | 0.5 |
| Core Tablet Total | 100.0 |

### Example 10: Comparison of Etrumadenant Tablets

Tablets comprising salts of etrumadenant were also prepared.

A crystalline form of a phosphate salt of etrumadenant (“Phosphate Salt Form I”) was prepared as follows. About 176 mg of etrumadenant was suspended in about 4 mL of water:acetone. Then, about 140 μL of 85% H₃PO₄ was added, and the sample left to sit at room temperature.

A crystalline form of a phosphate salt of etrumadenant (“Phosphate Salt Form 1”) may also be prepared as follows. About 5 g of etrumadenant was suspended in about 20 mL of tetrahydrofuran. Next, about 1.5 mL of 85% H₃PO₄ diluted in about 2 mL of water was added to the suspension. A solution formed, and the sample was seeded with seeds of Phosphate Salt Form I (made as described above). A suspension formed and was stirred overnight. The sample was filtered and washed with about 10 mL of water, and the solids dried at room temperature under vacuum with nitrogen sweep, yielding Phosphate Salt Form I.

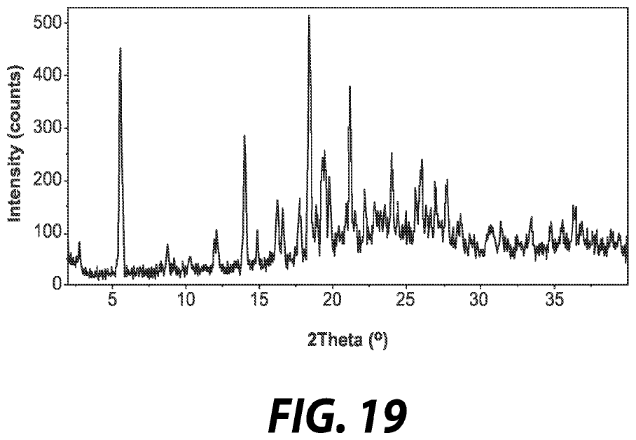

A crystalline form of a phosphate salt of etrumadenant (“Phosphate Salt Form I”) is characterized by an XRPD as shown in FIG. 19.

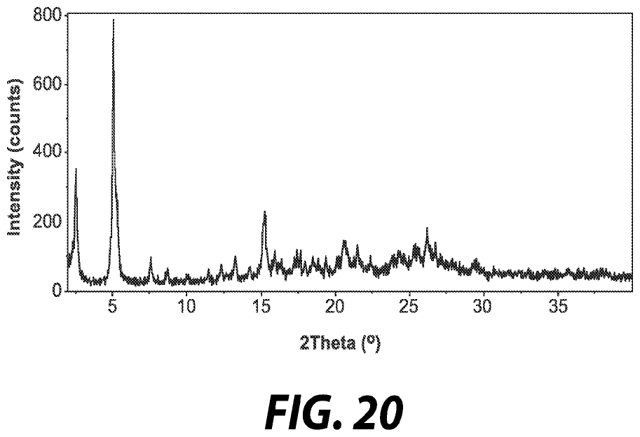

Another sample of a crystalline form of a phosphate salt of etrumadenant (“Phosphate Salt Form II”) was made as follows. About 120 mL of ethanol:water (9:1 vol) was added to a 250 mL container. To this, about 2 molar equivalents of phosphoric acid was added along with a magnetic stirrer. Next, about 6 grams of crystalline etrumadenant (Form I as described in WO 2020/018680) was added, and the mixture stirred at room temperature. About 2 additional equivalents of phosphoric acid was added to the mixture, and it was further stirred at room temperature. The resulting solids were filtered and washed twice with around 15 mL of ethanol:water (9:1 vol) having excess phosphoric acid. The sample was dried in funnel filter overnight, yielding a crystalline form of a phosphate salt of etrumadenant (“Phosphate Salt Form II”) characterized by an XRPD as shown in FIG. 20.

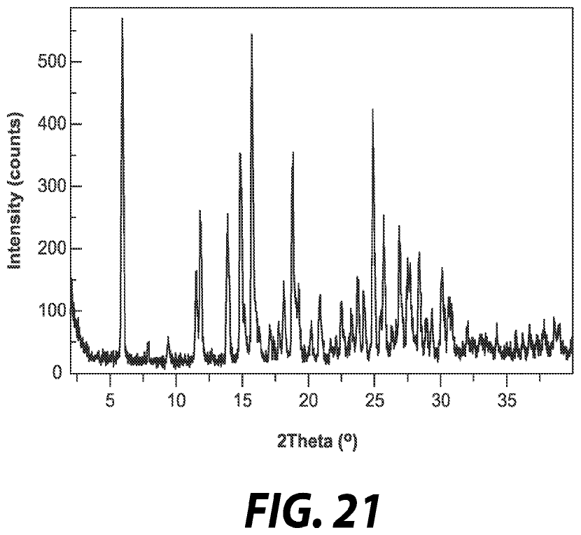

Etrumadenant fumaric acid salt was isolated when a 4 mL glass vial was charged with about 213 mg crystalline etrumadenant (Form I made as described in WO 2020/018680), about 70 mg of fumaric acid, and a few mL of tetrahydrofuran, forming a solution. The solution was allowed to evaporate at ambient temperature, and on the next day had become a brown oil. About 1 mL of methanol was added to the oil, resulting in a suspension. Solids were isolated and analyzed about one week later. The etrumadenant fumaric acid salt XRPD pattern is shown in FIG. 21.

Etrumadenant tablets were manually prepared to contain 50 mg etrumadenant-equivalence introduced as a phosphoric acid salt as described above or fumaric acid salt as described above. Tablets were prepared as follows. API was blended with intragranular components in a suitable blender. The blend was de-lumped and then subject to compaction and milled into granules. Extragranular components were then blended with the granules and the mixture subject to tableting. Tablet composition for the phosphoric acid salt is provided in Table 25 and for the fumaric acid salt is provided in Table 26.

TABLE 25
Etrumadenant Phosphoric Acid Salt Tablet Composition

| | |
|---|---|
| Material | Composition (% w/w) |

Intra-Granular

| | |
|---|---|
| AB928 phosphoric acid salt | 12.3^{a} |
| Microcrystalline cellulose | 40.9 |
| Mannitol | 40.8 |
| Croscarmellose sodium | 5.0 |
| Magnesium Stearate | 0.5 |

Extra-Granular

| | |
|---|---|
| Magnesium Stearate | 0.5 |
| Core Tablet Total | 100.0 |

^{a}Equivalent to 10.0% w/w etrumadenant with a theoretical salt correction factor of 0.813

TABLE 26
Etrumadenant Fumaric Acid Salt Tablet Composition

| | |
|---|---|
| Material | Composition (% w/w) |

Intra-Granular

| | |
|---|---|
| AB928 fumaric acid salt | 12.7^{a} |
| Microcrystalline cellulose | 39.3 |
| Mannitol | 42.0 |
| Croscarmellose sodium | 5.0 |
| Magnesium Stearate | 0.5 |

Extra-Granular

| | |
|---|---|
| Magnesium Stearate | 0.5 |
| Core Tablet Total | 100.0 |

^{a}Equivalent to 10.0% w/w etrumadenant with a theoretical salt correction factor of 0.787

Fasted, Pentagastrin Pretreated Beagle Dog Study: Pharmacokinetics (PK) of etrumadenant tablets were evaluated in male beagle dogs fasted overnight prior to study drug administration. Each animal received a single 6 μg/kg intramuscular injection of pentagastrin approximately 30 minutes prior to test article administration. Whole blood samples were collected pre-dose, and 0.25, 0.5, 1, 2, 4, 6, 8, 12, and 24 hours following administration (PO) of a single dose of study drug. Data is shown in Table 27. A crystalline form of a phosphate salt of etrumadenant (“Phosphate Salt Form I”) could not be reproduced and thus was not desirable. The tablet having a solid dispersion of etrumadenant afforded the highest oral bioavailability.

TABLE 27
PK Data in Fasted, Pentagastrin Pretreated Beagle Dogs

| | Effective | | |
|---|---|---|---|
| | Dose | AUClast | % |
| Formulation | (mg/kg) | (hr*ng/mL) | Bioavailability^{a} |
| SDD Tablet as | 4.4 | 16100 ± 1520 | 72.8 ± 6.9 |
| described in Table 24 | | | |
| Crystalline AB928 | 4.3 | 6400 ± 1970 | 29.7 ± 9.1 |
| Tablet as described in | | | |
| Table 2 | | | |
| Tablet as described in | 4.2 | 12200 ± 430 | 57.9 ± 2.0 |
| Table 25 with | | | |
| Phosphate Salt Form | | | |
| I | | | |
| Tablet as described in | 5.3 | 8690 ± 2770 | 32.7 ± 10.4 |
| Table 25 with | | | |
| Phosphate Salt Form | | | |
| II | | | |
| Tablet as described in | 5.0 | 14600 ± 4310 | 58.2 ± 17.2 |
| Table 26 | | | |

^{a}% Bioavailability is estimated based on AUClast of 25100 hr*ng/mL in beagle dogs when dose IV at 5 mg/kg.

Fasted, Famotidine Pretreated Beagle Dog Study (informed on potential drug-drug-interaction with acid suppressive agents): Pharmacokinetics (PK) of etrumadenant tablets were evaluated in male beagle dogs fasted overnight prior to study drug administration. Each animal received a single 20 mg tablet of famotidine approximately 1 hour prior to test article administration. Whole blood samples were collected pre-dose, and 0.25, 0.5, 1, 2, 4, 6, 8, 12, and 24 hours following administration (PO) of a single dose of study drug. Data is shown in Table 28. A crystalline form of a phosphate salt of etrumadenant (“Phosphate Salt Form I”) could not be reproduced and thus was not desirable while crystalline fumarate salt exhibited the lowest oral bioavailability. The tablet having a solid dispersion of etrumadenant afforded the highest oral bioavailability.

TABLE 28
PK Data in Fasted, Famotidine Pretreated Beagle Dogs

| | Effective | | |
|---|---|---|---|
| | Dose | AUClast | % |
| Formulation | (mg/kg) | (hr*ng/mL) | Bioavailability^{a} |
| SDD Tablet as | 5.4 | 15100 ± 2080 | 55.7 ± 7.7 |
| described in Table 24 | | | |
| Tablet as described in | 4.4 | 14300 ± 9980 | 64.8 ± 45.2 |
| Table 25 with | | | |
| Phosphate Salt Form | | | |
| I | | | |
| Tablet as described in | 5.5 | 6870 ± 4600 | 24.9 ± 16.7 |
| Table 25 with | | | |
| Phosphate Salt Form | | | |
| II | | | |
| Tablet as described in | 5.0 | 4050 ± 2800 | 16.1 ± 11.2 |
| Table 26 | | | |

^{a}% Bioavailability is estimated based on AUClast of 25100 hr*ng/mL in beagle dogs when dose IV at 5 mg/kg.

### Example 11: Pharmacokinetic Studies

This example describes a Phase 1 study to compare the single-dose PK of an etrumadenant tablet versus an etrumadenant capsule, in healthy adult participants (19-55 years of age), and to evaluate the effect of food on the single-dose PK of etrumadenant tablet, in healthy adult participants.

The study was an open-label, randomized, 3-treatment, 3 period crossover study to evaluate the relative bioavailability (BA) of etrumadenant tablet and capsule formulations and the effect of food on the tablet formulation. On Day 1 of each period, a single dose of etrumadenant was administered in a 3-period crossover fashion. Participants (n=24) received etrumadenant capsules under fasting (Treatment A) conditions and etrumadenant tablets under fasting (Treatment B) and fed (high-fat meal; Treatment C) conditions. PK samples for etrumadenant and its metabolites were taken predose and up to 120 hours post dose. There was a washout of at least 7 days between the etrumadenant doses. All participants who received at least one dose of etrumadenant (including participants who terminate the study early) were asked to return to the Clinical Research Unit (CRU) 14 (+2) days after the last dose for follow-up procedures, and to determine if any adverse event (AE) has occurred since the last study visit.

Study treatments are described as follows. Treatment A=150 mg etrumadenant (6×25 mg capsules) at Hour 0 on Day 1; participants fasted for at least 10 hours prior to etrumadenant dosing and for at least 4 hours post dose. Treatment B=150 mg etrumadenant (2×75 mg tablets) at Hour 0 on Day 1; participants fasted for at least 10 hours prior to etrumadenant dosing and for at least 4 hours post dose. Treatment C=150 mg etrumadenant (2×75 mg tablets) at Hour 0 on Day 1, 30 minutes after the start of a high-fat meal; participants fasted for at least 10 hours until 30 minutes prior to etrumadenant dosing when they were given a high-fat breakfast that was completely consumed within 30 minutes; participants then fasted for at least 4 hours post dose. The compositions of the etrumadenant capsule and etrumadenant tablet are provided in Table 3 and Table 20, respectively.

All etrumadenant capsules or tablets were administered orally with approximately 240 mL of water. If all the capsules and tablets could not be swallowed at the same time, up to a maximum of 50 mL of additional water were administered as required by the participant. Dosing was completed within 10 minutes.

Study objectives included pharmacokinetics (PK) and safety. The following plasma PK parameters were calculated for etrumadenant and its metabolites, as appropriate:
AUC0-24: The area under the concentration time curve, from time 0 to 24 hour post dose.
AUClast*: The area under the concentration-time curve, from time 0 to the last observed non-zero concentration.
AUCinf*: The area under the concentration time curve from time 0 extrapolated to infinity.
AUC % ex: Percent of AUCinf extrapolated.
CL/F: Apparent total plasma clearance after oral administration, (parent only).
Cmax*: Maximum observed concentration.
Tmax: Time to reach Cmax.
Tlag: Lag time, determined as the time point prior to the first observed/measured non-zero plasma concentration.
λ_{z}: Terminal elimination rate constant.
t½: Apparent terminal elimination half-life.
Vz/F: Apparent volume of distribution during terminal phase (parent only).
MP AUC0-24: Metabolite (m) to parent (p) molar ratio for AUC0-24 calculated for both metabolites.
MT AUC0-24: Metabolite (m) to total drug-related material (t) molar ratio for AUC0-24 calculated for both metabolites.
=Primary PK parameters

Additional PK parameters were calculated if deemed appropriate. Least-squares means (LSMs) ratios of “test/reference” in the In-transformed PK parameters (AUClast, AUCinf, and Cmax) were calculated with the 2-sided 90% confidence intervals (CI). The comparisons of interest included the following:
Relative BA of the tablet versus the capsule: The LSM ratio of tablet formulation fasted (Treatment B—test) compared to capsule formulation fasted (Treatment A—reference).
Effect of Food: The LSM ratio of tablet formulation fed (Treatment C—test) compared to tablet formulation fasted (Treatment B—reference).

A non-parametric Wilcoxon signed rank test may be performed on the variable Tmax for the test—reference differences and the results tabulated.

The relative bioavailability of the capsule versus the tablet is shown in Table 29. Etrumadenant Cmax, AUC and Tmax in capsules and tablets are statistically equivalent. Administration of etrumadenant under fed conditions had no effect on its overall exposure (AUClast and AUCINF) compared to fasting condition (Table 30).

TABLE 29
Relative bioavailability capsule v. tablet

| | Capsule | Tablet | | |
|---|---|---|---|---|
| | GeoLSM | GeoLSM | Ratio % | CI 90% |
| Cmax (ng/ml) | 2746 | 2664 | 97.01 | 91.48-102.87 |
| AUClast (ng*h/ml) | 36081 | 36050 | 99.92 | 94.76-105.35 |
| AUCINF (ng*h/ml) | 37939 | 37576 | 99.04 | 93.61-104.79 |

Both capsule and table median Tmax is 1.5 h
PK parameters based on nominal time.

TABLE 30
Food effect on tablet

| | Fasted | Fed | | |
|---|---|---|---|---|
| | GeoLSM | GeoLSM | Ratio % | CI 90% |
| Cmax (ng/ml) | 2664 | 2174 | 81.59 | 76.74-86.76 |
| AUClast (ng*h/ml) | 36050 | 39163 | 108.63 | 104.05-113.42 |
| AUCINF (ng*h/ml) | 375576 | 41271 | 109.83 | 105.26-114.61 |

Both capsule and table median Tmax is 1.5 h
PK parameters based on nominal time.

### Example 12: Drug-Drug Interaction Studies

An open-label, fixed-sequence, 2-period drug-drug interaction (DDI) study was designed to evaluate the effect of multiple doses of a strong inhibitor of CYP3A4 and P-gp on the single-dose PK of etrumadenant in humans. Etrumadenant was supplied as 25 mg capsules. The composition of the etrumadenant capsule is provided in Table 3. Itraconazole was used as the representative strong inhibitor of CYP3A4 and P-gp. It was supplied as 10 mg/mL oral solution, Sporanox® by Janssen Pharmaceuticals (or generic equivalent). Twenty (20) healthy, adult male and female (of non-childbearing potential) participants were enrolled. Screening of participants occurred within 28 days prior to the first dosing.

On Day 1 of Period 1, a single 150 mg dose of etrumadenant was orally administered under fasting conditions (i.e., at least 10 hours of fasting prior to dosing and at least 4 hours postdose). PK sampling for etrumadenant and its metabolites was taken predose and up to 120 hours postdose. There was a washout of 5 days between the etrumadenant dose in Period 1 and the first itraconazole dose in Period 2.

In Period 2, 200 mg itraconazole was orally administered twice a day (BID) on Day 1 and once a day (QD) for 9 consecutive days (on Days 2 through 10) with a single 150 mg dose of etrumadenant orally administered 1 hour after itraconazole dosing on Day 6. Itraconazole was administered under fasting conditions. On Day 6, participants fasted for at least 10 hours prior to and at least 4 hours post etrumadenant dosing. PK sampling for etrumadenant and its metabolites was taken predose and up to 120 hours after etrumadenant dosing. PK sampling for itraconazole and 1-hydroxy-itraconazole was collected on Days 5, 6, 7, 9, and 11.

Itraconazole was administered twice on the first day of Period 2 as a loading dose to accelerate CYP3A inhibition and the inhibition was then maintained by administering itraconazole 200 mg QD for 9 consecutive days. A 200 mg BID dose level is expected to provide similar inhibition to a 400 mg QD dose. Although itraconazole reaches steady state within approximately 15 days, it has been established that QD administration of 200 mg itraconazole for 3 to 5 days allows maximum CYP3A inhibition sufficient to detect a DDI. Itraconazole is also a P-gp inhibitor, and it has been reported in the literature that 200 mg QD administration of itraconazole for 5 days increased oral digoxin AUC by approximately 1.7-fold. As such, administration of itraconazole for at least 4 days is considered sufficient in DDI studies as a CYP3A4 and P-gp inhibitor. To maintain the level of inhibition, itraconazole was administered throughout PK sampling of etrumadenant (i.e., through Day 10 of Period 2). In addition, the itraconazole oral solution was administered under fasting conditions to maximize bioavailability.

Participants were housed on Day −1 of Period 1, at the time indicated by the clinical research unit (CRU), until after the 120-hour blood draw and completion of study procedures in Period 2. Safety was monitored throughout the study by repeated clinical and laboratory evaluations. In this study, there were no discontinued participants. All participants who received at least one dose of study drug (including participants who terminated the study early) were asked to return to the CRU approximately 14 days after the last dose for follow-up procedures, and to determine if any adverse event (AE) had occurred since the last study visit. All adverse events reported in the study were mild and resolved without treatment.

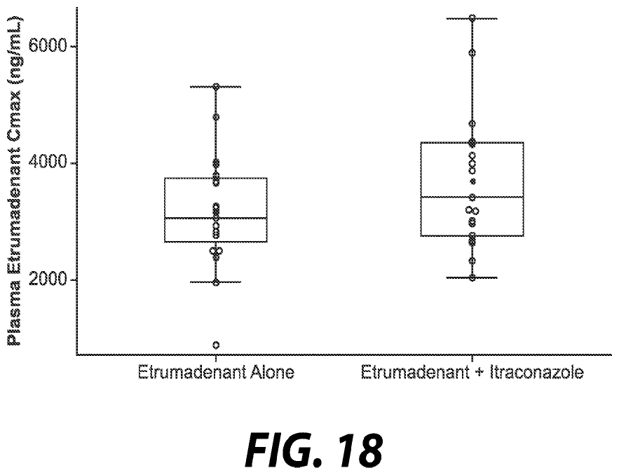

The summary of plasma etrumadenant PK parameters and box plots for Cₘₐₓ, AUCₗₐₛₜ, and AUC_{inf} with and without itraconazole am presented in Table 27 and in FIG. 18. Multiple doses of itraconazole resulted in an increase of approximately 63%, 62%, and 18% in etrumadenant AUClast, AUCinf, and Cmax, respectively, compared to etrumadenant given alone. The increase in etrumadenant AUC after co-administration of etrumadenant and itraconazole indicates etrumadenant metabolism is inhibited by itraconazole via CYP3A4 in humans. The magnitude of increase (62-63%) suggests etrumadenant is neither a moderately sensitive nor sensitive substrate of CYP3A4 per US Food and Drug Administration (FDA) drug-interaction guidance (2020). The minimal increase (18%) in etrumadenant Cₘₐₓ suggests coadministration of a P-gp inhibitor (itraconazole) with etrumadenant does not cause meaningful exposure alteration of etrumadenant peak concentration.

Multiple-doses of a strong CYP3A4 and P-gp inhibitor (itraconazole) resulted in an increase in the metabolite-to-parent ratios of MP AUC0-24 and MP Cmax for a etrumadenant glucuronide metabolite, compared to etrumadenant given alone. The MP AUC0-24 and MP Cmax values increased approximately 8% and 6%, respectively, with coadministration of itraconazole. The limited changes in metabolite-to-parent ratio are consistent with etrumadenant glucuronide being a primary metabolite formed by direct glucuronidation of etrumadenant. Consequently, coadministration of a CYP3A4 or a P-gp inhibitor will have little effect on the fraction of etrumadenant eliminated via glucuronidation pathway.

Multiple-doses of a strong CYP3A4 and P-gp inhibitor (itraconazole) resulted in a decrease in the metabolite-to-parent ratios of MP AUC0-24 and MP Cmax for the N-dealkylated etrumadenant, compared to etrumadenant given alone. The MP AUC0-24 and MP Cmax values decreased approximately 87% and 90%, respectively, with coadministration of itraconazole. The decrease in formation of N-dealkylated etrumadenant is consistent with the inhibitory effect on the CYP3A4 elimination pathway by itraconazole, suggesting etrumadenant is eliminated by CYP3A4 pathway via formation of N-dealkylated etrumadenant.

The total percent change of exposure in drug-related material in plasma is less than 26% and 7% for AUC0-24 and Cmax, respectively, in the presence and absence of itraconazole. This change is not clinically meaningful. Thus, the absorption of etrumadenant is not affected by coadministration of a P-gp inhibitor (itraconazole) with etrumadenant.

In summary, the effect of a strong CYP3A4 inhibitor on the PK of etrumadenant is limited; and P-gp may not influence etrumadenant's oral absorption. Physiologically-based pharmacokinetic modelling suggested that the fraction of etrumadenant metabolized through CYP3A4 is approximately 0.4.

TABLE 31

| Pharmacokinetic | Etrumadenant | | Etrumadenant + | |
|---|---|---|---|---|
| Parameters | Alone N = 20 | | Itraconazole N = 19 | |
| AUC0-24 (ng*hr/mL) | 21600 | (31.3) | 30700 | (26.7) |
| AUClast (ng*hr/mL) | 36500 | (25.0) | 59400 | (32.6) |
| AUCinf (ng*hr/mL) | 38600 | (24.3) | 62600 | (33.9) |
| AUC % ex (%) | 3.90 | (97.8) | 4.39 | (58.4) |
| Cmax (ng/mL) | 3000 | (39.1) | 3530 | (31.3) |
| Tmax (hr) | 1.38 | (0.99, 2.50) | 1.52 | (1.00, 3.00) |

| | | |
|---|---|---|
| Kel (1/hr) | 0.0249 ± 0.00663 | 0.0255 ± 0.00605 |
| t½ (hr) | 29.9 ± 8.88 | 28.7 ± 7.01 |
| CL/F (L/hr) | 4.00 ± 1.12 | 2.52 ± 0.842 |
| Vz/F (L) | 172 ± 64.8 | 100 ± 28.3 |

Etrumadenant Alone: 150 mg etrumadenant (6 × 25 mg capsules) at Hour 0 on Day 1 (Period 1)
Etrumadenant + Itraconazole: 200 mg itraconazole (20 mL of 10 mg/mL oral solution) BID on Day 1; 200 mg itraconazole (20 mL of 10 mg/mL oral solution) QD on Days 2-10, with 150 mg etrumadenant (6 × 25 mg capsules) co-administered at Hour 0 on Day 6, 1 hour after itraconazole dosing (Period 2)
Subject 17 (Etrumadenant + Itraconazole) was not administered itraconazole on Days 8, 9, and 10 due to multiple days of high bilirubin levels. The subject's Treatment B data were excluded from the PK analyses.
Kel = elimination rate constant.
AUCs and Cmax are presented as geometric mean (geometric CV %).
Tmax is presented as median (minimum, maximum).
Other parameters are presented as arithmetic mean ± SD.

We claim:

1. A solid dispersion comprising about 20% to about 40% (w/w) of etrumadenant and about 60% to about 80% (w/w) of a polymer selected from hydroxypropylmethylcellulose acetate succinate (HPMCAS), copovidone (PVP-VA), cellulose acetate phthalate (CAP), hydroxypropyl methylcellulose E3 (HPMC E3), hydroxypropyl methylcellulose phthalate (HPMCP), polyvinylpyrrolidone (PVP), and polyvinyl caprolactam-polyvinyl acetate-polyethylene glycol graft copolymer (Soluplus®).

2. A solid dispersion comprising about 20% to about 40% (w/w) of etrumadenant and about 60% to about 80% (w/w) of a polymer (w/w) selected from hydroxypropylmethylcellulose acetate succinate (HPMCAS) and copovidone (PVP-VA).

3. The solid dispersion of claim 1 or 2, wherein the solid dispersion comprises
about 25% to about 35% (w/w) of etrumadenant; or
about 25% to about 30% (w/w) of etrumadenant.

4. The solid dispersion of claim 1 or 2, wherein the solid dispersion comprises
about 25% to about 35% (w/w) of etrumadenant and about 65% to about 75% of the polymer, or
about 25% to about 30% (w/w) of etrumadenant and about 70% to about 75% (w/w) of the polymer.

5. The solid dispersion of claim 1 or claim 2, wherein the solid dispersion comprises
about 22.5% to about 27.5% (w/w) of etrumadenant; or
about 27% to about 33% (w/w) of etrumadenant; or
about 31.5% to about 38.5% (w/w) of etrumadenant.

6. The solid dispersion of claim 5, wherein the solid dispersion is characterized by a single glass transition temperature (T_{g}).

7. The solid dispersion of claim 6, wherein the solid dispersion is characterized by
a glass transition temperature (T_{g}) from about 75° C. to about 100° C.; or
a degree of crystallinity that is no more than 5%.

8. The solid dispersion of claim 6, wherein the solid dispersion is characterized by
a glass transition temperature (T_{g}) from about 75° C. to about 100° C.; or
a degree of crystallinity that is no more than 1%.

9. The solid dispersion of claim 6, wherein the solid dispersion is characterized by
a single melting temperature (Tₘ); or
a diffraction pattern by x-ray powder diffraction (XRPD) that is absent of discrete peaks.

10. The solid dispersion of any one of claims 1 to 2, wherein the polymer is HPMCAS.

11. The solid dispersion of claim 10, wherein the HPMCAS comprises an acetyl content of about 5% to about 14%, a succinyl content of about 4% to about 18%, a methoxyl content of about 20% to about 26%, and a hydroxypropoxy content of about 5% to about 10%.

12. The solid dispersion of claim 10, wherein the HPMCAS comprises an acetyl content of about 7% to about 11%, a succinyl content of about 10% to about 14%, a methoxyl content of about 21% to about 25%, and a hydroxypropoxy content of about 5% to about 9%.

13. The solid dispersion of any one of claims 10-12, wherein the solid dispersion comprises
about 22.5% to about 27.5% (w/w) of etrumadenant; or
about 27% to about 33% (w/w) of etrumadenant; or
about 31.5% to about 38.5% (w/w) of etrumadenant.

14. The solid dispersion of any one of claims 10-13, wherein the solid dispersion is characterized by a single Ts.

15. The solid dispersion of claim 14, wherein the solid dispersion is characterized by
a single glass transition temperature (T_{g}) from about 75° C. to about 85° C.; or
a degree of crystallinity that is no more than 5%.

16. The solid dispersion of claim 14, wherein the solid dispersion is characterized by
no other melt or crystallization events; or
a diffraction pattern by XRPD that is absent of discrete peaks.

17. The solid dispersion of any one of the claims 10-16, wherein the solid dispersion is formed by spray-drying.

18. The solid dispersion of any one of the preceding claims, wherein the solid dispersion has at least a 3-fold increase in AUC₃₋₂₁₀ FaSSIF (min*μgA/mL) compared to crystalline etrumadenant by non-sink dissolution testing.

19. The solid dispersion of claim 18, wherein the solid dispersion has about a 4-fold, 5-fold, 6-fold, or 7-fold increase in AUC₃₅₋₂₁₀ FaSSIF (min*μgA/mL) compared to crystalline etrumadenant by non-sink dissolution testing.

20. The solid dispersion of any one of the preceding claims, wherein the solid dispersion has a total impurity amount of less than 1% (% area), measured by HPLC.

21. The solid dispersion of any one of the preceding claims, wherein upon storage of the solid dispersion at 25° C. and 60% relative humidity in a closed packaging for 6 months, 12 months, 18 months, 24 months, or 36 months:
(i) the amount of etrumadenant in the solid dispersion is about 90% to about 110% of the value measured at 0 months;
(ii) the solid dispersion has a total impurity amount of less than or equal to 2%, or less than or equal to 1% (% area), measured by high-performance liquid chromatography (HPLC);
(iii) the solid dispersion is characterized by a single T_{g}, and optionally a single melting temperature (Tₘ), by modulated differential scanning calorimetry (MDSC);
(iv) the solid dispersion is characterized by a diffraction pattern by XRPD that is absent of discrete peaks; or
(v) any combination of (i) to (iv).

22. Spray-dried particles comprising a solid dispersion according to any one of claim 1 to 21, wherein the spray-dried particles are characterized by a Dv90 of less than 150 μm or a Dv50 of less than 50 sm.

23. The spray-dried particles of claim 22, wherein the spray-dried particles are characterized by
a Dv90 of about 80 μm to about 130 μm; or
a Dv50 of about 25 μm to about 40 μm.

24. A dosage form comprising a solid dispersion according to any one of claim 1 to 21 or spray dried particles of claims 22-23.

25. The dosage form of claim 24, wherein the dosage form is a tablet or capsule.

26. A granule comprising a solid dispersion according to any one of claims 1 to 21 and one or more fillers, one or more disintegrants, optionally one or more glidants, and optionally one or more lubricants.

27. A composition comprising a solid dispersion according to any one of claims 1 to 21 and one or mor fillers, one or more disintegrants, optionally one or more glidants, and optionally one or more lubricants.

28. The composition of claim 27, wherein the composition comprises about 40% to about 60% r (w/w) of the solid dispersion.

29. The composition of claim 27, wherein the composition comprises about 45% to about 50% (w/w) of the solid dispersion.

30. The composition of claim 27, wherein the composition comprises about 35% to about 45% (w/w) of the solid dispersion, about 40% to about 50% (w/w) of the one or more fillers, about 2% to about 7% (w/w) of the one or more disintegrants, and about 0.25% to about 0.75% (w/w) of the one or more lubricants.

31. The composition of claim 27, wherein the composition comprises about 47% (w/w) of the solid dispersion, about 43% to about 47% (w/w) of the one or more fillers, about 4% to about 6% (w/w) of the one or more disintegrants, about 0.5 to about 1% (w/w) of the one or more glidants, and less than 1% (w/w) of the one or more lubricants.

32. The composition of claim 27, wherein the composition comprises about 47.3% (w/w) of the solid dispersion, about 47.3% (w/w) of the one or more fillers, about 4.1% (w/w) of the one or more disintegrants, about 0.6% (w/w) of the one or more glidants, and about 0.6% (w/w) of the one or more lubricants.

33. The composition of claim 27, wherein the composition comprises about 50% (w/w) of the solid dispersion, about 17.5% (w/w) of the one or more tillers, about 31.8% (w/w) of the one or mor disintegrants, and about 0.6% (w/w) of the one or more lubricants.

34. The composition of any one of claims 27 to 33, wherein
the one or more fillers is selected from anhydrous calcium hydrogen phosphate, calcium hydrogen phosphate dihydrate, tribasic calcium phosphate, guar gum, lactose monohydrate, mannitol, sorbitol, microcrystalline cellulose (MCC), co-processed MCC, silicified microcrystalline cellulose, and mesoporous silica; or
the one or more disintegrants is croscarmellose sodium; or
the one or more lubricants, if present, is sodium stearyl fumarate or magnesium stearate; or
the one or more glidant, if present, is colloidal silica.

35. The composition of claim 34, wherein
the one or more fillers is selected from MCC, mannitol, silicified MCC, and mesoporous silica; or
the one or more disintegrants is croscarmellose sodium; or
the one or more lubricants, if present, is sodium stearyl fumarate or magnesium stearate; or
the one or more glidants, if present, is colloidal silica.

36. The composition of claim 32, comprising a first filler and a second filler, and wherein the solid dispersion, first filler, and second filler have a 2:1:1 weight ratio.

37. The composition of claim 30 or claim 35, wherein a first filler is MCC, and a second filler is mannitol.

38. The composition of claim 36 or 37, wherein
the one or more disintegrants is croscarmellose sodium; or
the one or more lubricants is sodium stearyl fumarate; or
the one or more glidants is colloidal silica.

39. The composition of claim 36 or 37, wherein
the one or more disintegrants is croscarmellose sodium;
the one or more lubricants is sodium stearyl fumarate; and
the one or more glidants is colloidal silica.

40. The composition of claim 37, wherein the one or more disintegrants is croscarmellose sodium; or the one or more lubricants is magnesium stearate.

41. A pharmaceutical composition comprising an intragranular component comprising a composition of any one of claims 27 to 40, and an extragranular component comprising one or more fillers, optionally one or more disintegrants, optionally one or more glidants, and one or more lubricants.

42. The pharmaceutical composition of claim 41, wherein the pharmaceutical composition comprises about 85% to about 95% (w/w) of the intragranular component, or about 85% (w/w) of the intragranular component.

43. The pharmaceutical composition of claim 41 or 42, wherein the extragranular component comprises: about 10% to about 15% (w/w) of the one or more fillers, about 1% to about 2% (w/w) of the one or more disintegrants, about 0.5% to about 1.0% (w/w) of the one or more glidants, and about 0.5% to about 1.0% (w/w) of the one or more lubricants.

44. The pharmaceutical composition of claim 41 or 42, wherein the extragranular component comprises: about 5% to about 15% (w/w) of the one or more fillers and about 0.25% to about 0.75% (w/w) of the one or more lubricants.

45. The pharmaceutical composition of claim 42, wherein
the one or more fillers is selected from anhydrous calcium hydrogen phosphate, calcium hydrogen phosphate dihydrate, tribasic calcium phosphate, guar gum, lactose monohydrate, mannitol, sorbitol, microcrystalline cellulose, co-processed microcrystalline cellulose, silicified microcrystalline cellulose, and mesoporous silica; or
the one or more disintegrants, if present, is croscarmellose sodium: or
the one or more lubricants is sodium stearyl fumarate or magnesium stearate; or
the one or more glidants, if present, is colloidal silica.

46. The pharmaceutical composition of claim 43, wherein
the one or more fillers is selected from microcrystalline cellulose, mannitol, silicified microcrystalline cellulose, and mesoporous silica; or
the one or more disintegrants is croscarmellose sodium; or
the one or more lubricants is sodium stearyl fumarate; or
the one or more glidants is colloidal silica.

47. The pharmaceutical composition of claim 44, wherein the one or more fillers is selected from microcrystalline cellulose and mannitol, and the one or more lubricants is magnesium stearate.

48. The pharmaceutical composition of any one of claims 41 to 47, wherein the pharmaceutical composition comprises two fillers, optionally in a 1:1 to a 2:1 ratio.

49. The pharmaceutical composition of claim 41, wherein the composition is formulation of Table 16, Table 19, Table 20, or Table 24.

50. A tablet comprising microcrystalline cellulose (MCC), mannitol, croscarmellose sodium, colloidal silica, sodium stearyl fumarate, and a solid dispersion according to any one of claims 1 to 21.

51. The tablet of claim 50, wherein the tablet comprises about 25% to about 60% (w/w) of the solid dispersion.

52. The tablet of claim 50, wherein the tablet comprises about 35% to about 55% (w/w) of the solid dispersion.

53. The tablet of any one of claims 50 to 52, wherein the solid dispersion is according to any one of claims 10 to 21.

54. The tablet of claim 53, wherein the solid dispersion is according to claim 17.

55. The tablet of any one of claims 50 to 54, wherein the tablet comprises about 40% to about 50% (w/w) of the solid dispersion, about 10% to 25% (w/w) of MCC, about 20% to about 25% (w/w) of mannitol, about 5% to about 6% (w/w) of croscarmellose sodium, about 1% (w/w) of colloidal silica, and about 1% (w/w) of sodium stearyl fumarate.

56. A tablet comprising microcrystalline cellulose (MCC), mannitol, croscarmellose sodium, magnesium stearate, and a solid dispersion according to any one of claims 1 to 21.

57. The tablet of claim 56, wherein the tablet comprises about 35% to about 45% (w/w) of the solid dispersion, about 30% to about 42% (w/w) of MCC, about 13% to about 23% (w/w) of mannitol, about 2% to about 8% (w/w) croscarmellose sodium, and about 0.5% to about 1.5% (w/w) magnesium stearate.

58. The tablet of claim 50, wherein the tablet is a tablet of Table 16, Table 19, Table 20, or Table 24.

59. The tablet of any one of claims 50-58, wherein the tablet has a weight of about 100 mg to about 1 g.

60. The tablet of claim 59, wherein the tablet has a weight of about 100 mg, about 250 mg, about 500 mg, or about 750 mg.

61. The tablet of claim 59, wherein the tablet has a weight of 750 mg to 800 mg, 760 mg to 790 mg, 770 mg to 780 mg, 775 mg to 785 mg, or 780 mg to 790 mg.

62. The tablet of any one of claims 50-61, further comprising a coating.

63. The tablet of claim 62, wherein the coating is a non-functional coating.

64. The tablet of any one of claims 50-63, wherein the percent of etrumadenant released at 45 minutes is not less than 85% as measured by the dissolution method of Table 21.

65. The tablet of any one of claims 50-64, wherein upon storage at 25° C. and 60% relative humidity in a closed packaging for 6 months, 12 months, 18 months, 24 months, or 36 months:
(i) the amount of etrumadenant in the tablet is about 90 to about 110% of the value measured at 0 months;
(ii) a total impurity amount of less than or equal to 2%, less than or equal to 1%, or less than or equal to 0.5% (% area), measured by HPLC; or
(iii) any combination of (i) to (ii).

66. The tablet of any one of claims 50-65, wherein upon administration of the tablet to a human in a fed or fasted state, the human's overall exposure to etrumadenant is equivalent.

67. A process for preparing a solid dispersion comprising etrumadenant, the process comprising
mixing etrumadenant, a polymer selected from HPMCAS and copovidone, and a solvent to produce a spray solution; and
spray drying the spray solution to produce the solid dispersion.

68. The process of claim 67, wherein the weight ratio of etrumadenant to polymer is 25:75 to 40:60.

69. The process of claim 67 or 68, wherein the spray solution is prepared at 8% to 13% solids loading.

70. The process of any one of claim 67 to 69, wherein the spray drying step comprises atomizing the spray solution into a drying chamber that has an outlet temperature of about 38° C. to about 46° C., a gas to liquid ratio of about 0.5 to about 0.7, and a relative saturation (total) of about 15% to about 23%.

71. The process of any one of claim 67 to 70, wherein the solid dispersion undergoes further drying to produce a dried powder with a water content of less than about 1% (w/w).

72. A method of treating a disease, disorder, or condition, mediated at least in part by the adenosine A_{2A} receptor (A_{2A}R) and/or the adenosine A_{2B} receptor (A_{2B}R), comprising administering a dosage form of claim 24 or claim 25, a pharmaceutical composition of any one of claims 41 to 49, or a tablet of any one of claims 50 to 66 to a subject in need thereof.

73. The method of claim 72, wherein the disease, disorder, or condition is mediated at least in part by AAR.

74. The method of claim 72, wherein the disease, disorder, or condition is mediated at least in part by A_{2B}R.

75. The method of claim 72, wherein the disease, disorder, or condition is mediated at least in part by both the A_{2A}R and A_{2B}R receptors.

76. A method of treating cancer comprising administering a dosage form of claim 24 or claim 25, a pharmaceutical composition of any one of claims 41 to 49, or a tablet of any one of claims 50 to 66 to a subject in need thereof.

77. A method of treating cancer in a subject, said method comprising administering to said subject a dosage form of claim 24 or claim 25, a pharmaceutical composition of any one of claims 41 to 49, or a tablet of any one of claims 50 to 66; and at least one additional therapeutic agent; to a subject in need thereof.

78. The method of claim 77, wherein the cancer is a solid tumor.

79. The method of claim 77, wherein the cancer is breast cancer, lung cancer, gastrointestinal cancer, genitourinary cancer, or gynecological cancer.

80. The method of claim 77, wherein the cancer is bladder cancer, breast cancer, colorectal cancer, gastric cancer, gastroesophageal cancer, lung cancer, ovarian cancer, pancreatic cancer, or prostate cancer.

81. The method of claim 77, wherein the cancer is castrate resistant prostate cancer, esophageal adenocarcinoma, non-small cell lung carcinoma, pancreatic ductal adenocarcinoma, prostate adenocarcinoma, or urothelial cancer.

82. The method of any one of claims 72 to 81, wherein the subject is administered a total daily dosage of about 50 mg etrumadenant to about 250 mg etrumadenant, or about 50 mg etrumadenant to about 150 mg etrumadenant.

83. The method of claim 82, wherein the subject is administered a total daily dosage of about 50 mg, about 75 mg, about 100 mg, about 125 mg, or about 150 mg.

84. A method of treating cancer in a subject concomitantly receiving a CYP3A4 inhibitor or a P-gp inhibitor or in a subject that is a poor CYP3A4 metabolizer, said method comprising administering to said subject a dosage form of claim 24 or claim 25, a pharmaceutical composition of any one of claims 41 to 49, or a tablet of any one of claims 50 to 66 to a subject in need thereof.

85. A method of treating a disease, disorder, or condition, mediated at least in part by the adenosine A_{2A} receptor (A_{2A}R) or the adenosine A_{2B} receptor (A_{2B}R) in a patient, wherein said patient is concomitantly receiving a CYP3A4 inhibitor or a P-gp inhibitor or the subject is a poor CYP3A4 metabolizer, the method comprising administering a therapeutically effective amount of etrumadenant.
